(19) United States
(12) Patent Application Publication
(10) Pub. No.: US 2024/0155941 A1
(43) Pub. Date: May 9, 2024

(54) LIGHT EMITTING ELEMENT AND NITROGEN-CONTAINING COMPOUND FOR THE SAME

(71) Applicant: Samsung Display Co., Ltd., Yongin-si (KR)

(72) Inventors: HYEONGMIN KIM, Yongin-si (KR); YOUNGJIN PARK, Yongin-si (KR); HEECHOON AHN, Yongin-si (KR); HYUNAH UM, Yongin-si (KR); Juhui YUN, Yongin-si (KR); YESEUL LEE, Yongin-si (KR)

(21) Appl. No.: 18/474,995

(22) Filed: Sep. 26, 2023

(30) Foreign Application Priority Data
Oct. 5, 2022 (KR) 10-2022-0127378

(51) Int. Cl. H10K 85/60; C07D 405/14; C09K 11/06

(52) U.S. Cl. CPC H10K 85/6572; C07D 405/14; C09K 11/06; H10K 85/654; H10K 50/12

(57) ABSTRACT

A light emitting element including a first electrode, a second electrode on the first electrode, and an emission layer between the first electrode and the second electrode that includes a first compound represented by Formula 1. The light emitting element including the first compound of an embodiment may have a decrease in the driving voltage and exhibit excellent or suitable efficiency.

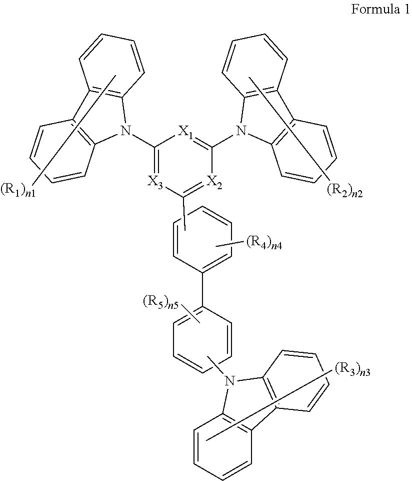

### CROSS-REFERENCE TO RELATED APPLICATION

This application claims priority to and the benefit of Korean Patent Application No. 10-2022-0127378, filed on Oct. 5, 2022, the entire content of which is hereby incorporated by reference in its entirety.

### BACKGROUND

### 1. Field

Embodiments of the present disclosure herein relate to a light emitting element and a nitrogen-containing compound utilized therein.

### 2. Description of the Related Art

Recently, the development of an organic electroluminescence display device as an image display device is being actively conducted. The organic electroluminescence display device includes so-called a self-luminescent light emitting element in which holes and electrons injected from a first electrode and a second electrode recombine in an emission layer, and thus a luminescent material of the emission layer emits light to implement display.

In the application of a light emitting element to a display device, there is a demand for a light emitting element having a decrease in the driving voltage and improvement in the light efficiency, and development on materials for a light emitting element capable of stably attaining such characteristics is being continuously required.

### SUMMARY

Aspects of embodiments are directed toward a light emitting element having a decrease in the driving voltage and improvement in the light efficiency.

Aspects of embodiments are directed toward a nitrogen-containing compound which is a material for a light emitting element having a decrease in the driving voltage and improvement in the light efficiency.

Additional aspects will be set forth in part in the description which follows and, in part, will be apparent from the description, or may be learned by practice of the presented embodiments of the disclosure.

According to one or more embodiments of the present disclosure, a light emitting element includes: a first electrode; a second electrode disposed on the first electrode; and an emission layer which is disposed between the first electrode and the second electrode and includes a first compound represented by Formula 1:

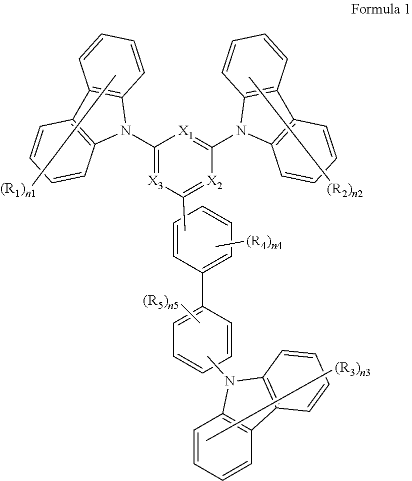

In Formula 1, at least one among X₁ to X₃ is N, the rest may each independently be CR₆, n1 to n3 may each independently be an integer of 0 to 8, n4 and n5 may each independently be an integer of 0 to 4, and R₁ to R₆ may each independently be a hydrogen atom, a deuterium atom, a halogen atom, a hydroxy group, a cyano group, a nitro group, a substituted or unsubstituted amine group, a substituted or unsubstituted oxy group, a substituted or unsubstituted thio group, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted aryl group having 6 to 60 ring-forming carbon atoms, or a substituted or unsubstituted heteroaryl group having 2 to 60 ring-forming carbon atoms.

In an embodiment, Formula 1 may be represented by Formula 1-1 or Formula 1-2:

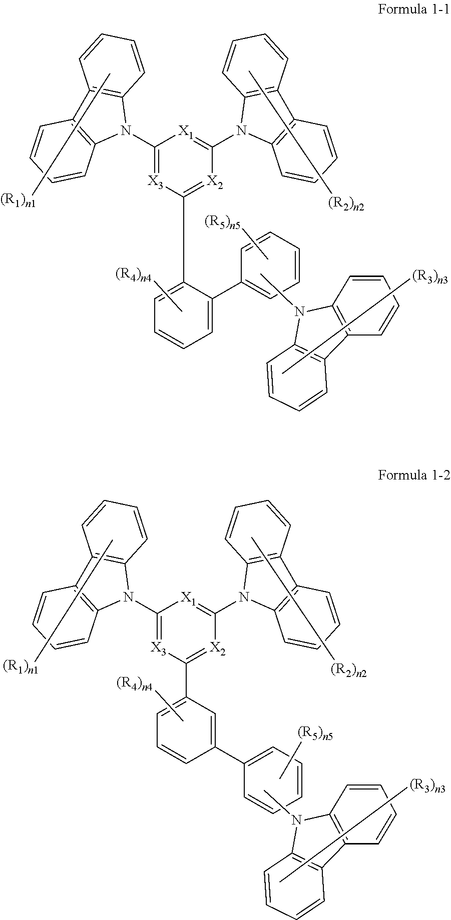

In Formula 1-1 and Formula 1-2, X₁ to X₃, n1 to n5, and R₁ to R₅ may each independently be the same as defined in Formula 1.

In an embodiment, Formula 1-1 may be represented by Formula 1-1A or Formula 1-B:

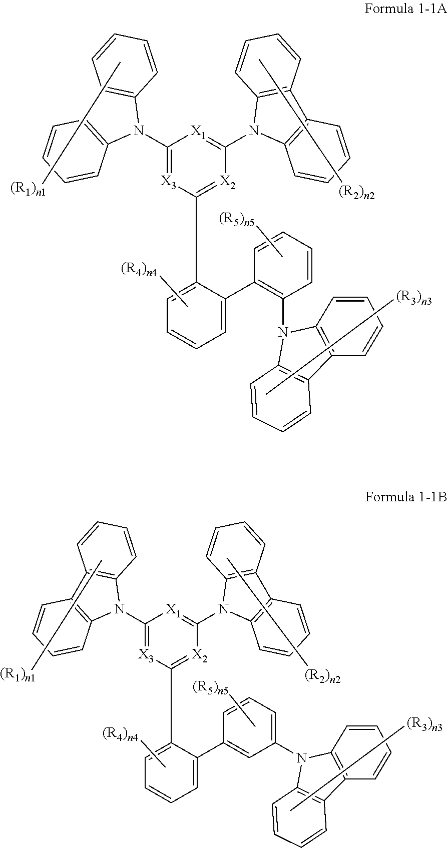

In Formula 1-1A to Formula 1-1B X₁ to X₃, n1 to n5, and R₁ to R₅ may each independently be the same as defined in Formula 1.

Formula 1-2 may be represented by Formula 1-2A or Formula 1-2B:

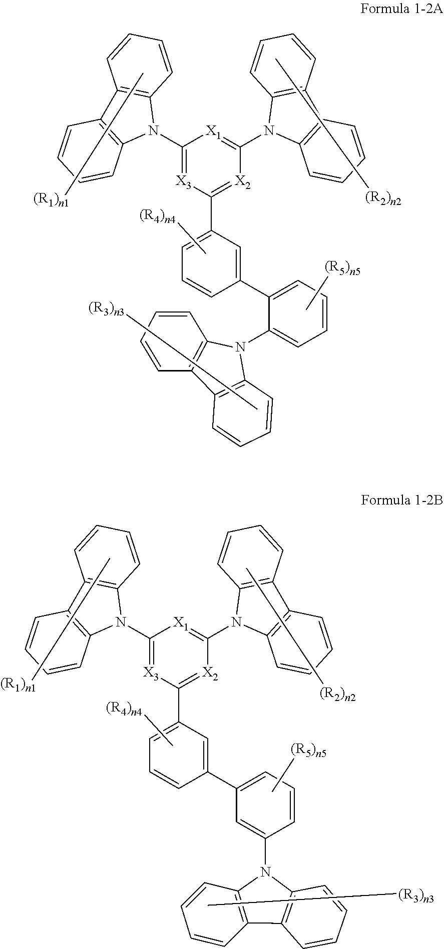

In Formula 1-2A to Formula 1-2B, X₁ to X₃, n1 to n5, and R₁ to R₅ may each independently be the same as defined in Formula 1.

In an embodiment, Formula 1 may be represented by any one among Formula 1-3 to Formula 1-5:

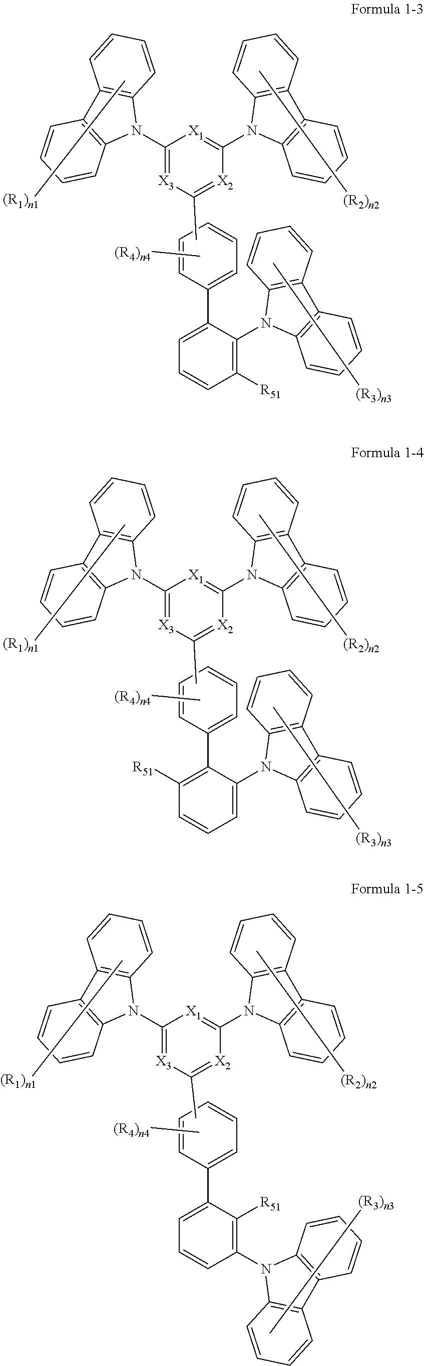

In Formula 1-3 to Formula 1-5, R₅₁ is a substituted or unsubstituted phenyl group, a substituted or unsubstituted carbazole group, a substituted or unsubstituted dibenzofuran group, or a substituted or unsubstituted dibenzothiophene group, and X₁ to X₃, n1 to n4, and R₁ to R₄ may each independently be the same as defined in Formula 1.

In an embodiment, in Formula 1, R₁ to R₆ may each independently be a hydrogen atom, a deuterium atom, or represented by any one among R-1 to R-7:

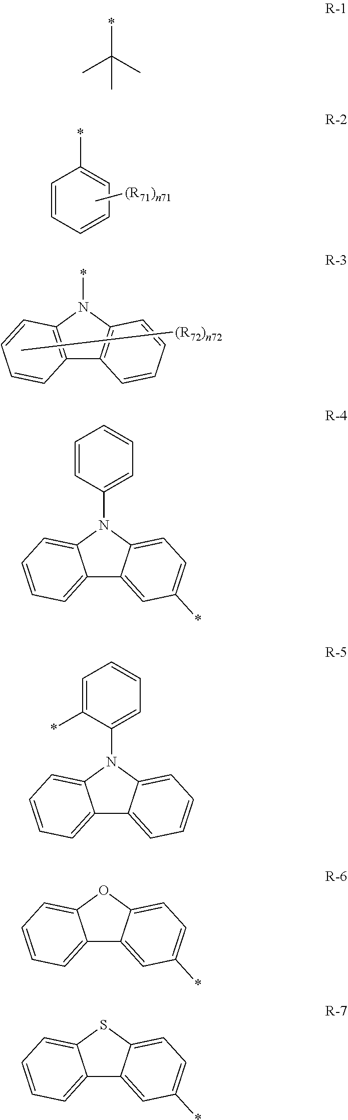

In R-2 and R-3, n71 is an integer of 0 to 5, n72 is an integer of 0 to 8, and R₇₁ and R₇₂ may each independently be a hydrogen atom, a deuterium atom, or an unsubstituted t-butyl group.

In an embodiment, at least one among R₁ to R₅ in Formula 1 may be a deuterium atom, or may include a substituent containing a deuterium atom.

In an embodiment, the emission layer may include a host and a dopant, and the host may include the first compound.

In an embodiment, the host may further include a second compound represented by Formula HT-1:

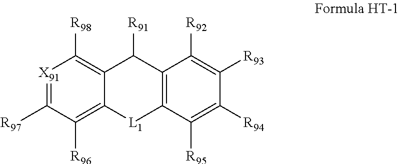

In Formula HT-1, L₁ is a direct linkage, CR₉₉R₁₀₀, or SiR₁₀₁R₁₀₂, X₉₁ is N or CR₁₀₃, and R₉₁ to R₁₀₃ may each independently be a hydrogen atom, a deuterium atom, a cyano group, a substituted or unsubstituted silyl group, a substituted or unsubstituted oxy group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, and/or are bonded to an adjacent group to form a ring.

In an embodiment, the dopant may include a third compound represented by Formula M-a:

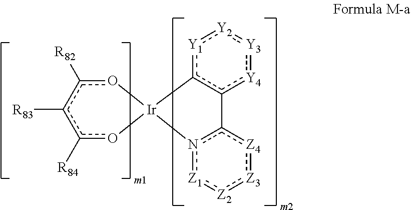

In Formula M-a, Y₁ to Y₄ and Z₁ to Z₄ may each independently be CR₈₁ or N, R₈₁ to R₈₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or are bonded to an adjacent group to form a ring, m1 is 0 or 1, and m2 is 2 or 3.

In an embodiment of the present disclosure, a nitrogen-containing compound is represented by Formula 1.

In an embodiment, in Formula 1, each of X₁ to X₃ may be N.

### BRIEF DESCRIPTION OF THE FIGURES

The accompanying drawings are included to provide a further understanding of the present disclosure, and are incorporated in and constitute a part of this specification. The drawings illustrate example embodiments of the present disclosure and, together with the description, serve to explain principles of the present disclosure.

In the drawings:

FIG. 1 is a plan view illustrating a display device according to an embodiment of the present disclosure;

FIG. 2 is a cross-sectional view illustrating a part taken along line I-I′ of FIG. 1;

FIG. 3 is a cross-sectional view schematically illustrating a light emitting element of an embodiment of the present disclosure;

FIG. 4 is a cross-sectional view schematically illustrating a light emitting element of an embodiment of the present disclosure;

FIG. 5 is a cross-sectional view schematically illustrating a light emitting element of an embodiment of the present disclosure;

FIG. 6 is a cross-sectional view schematically illustrating a light emitting element of an embodiment of the present disclosure;

FIG. 7 is a cross-sectional view illustrating a display device according to an embodiment of the present disclosure;

FIG. 8 is a cross-sectional view illustrating a display device according to an embodiment of the present disclosure;

FIG. 9 is a cross-sectional view illustrating a display device according to an embodiment of the present disclosure; and

FIG. 10 is a cross-sectional view illustrating a display device according to an embodiment of the present disclosure.

### DETAILED DESCRIPTION

The present disclosure may be modified in one or more suitable manners and have many forms, and thus specific embodiments will be exemplified in the drawings and described in more detail in the detailed description of the present disclosure. It should be understood, however, that it is not intended to limit the present disclosure to the particular forms disclosed, but rather, is intended to cover all modifications, equivalents, and alternatives falling within the spirit and scope of the present disclosure.

In the present specification, when a component (or a region, a layer, a portion, etc.) is referred to as being “on,” “connected to,” or “coupled to” another component, it refers to that the component may be directly disposed on/connected to/coupled to the other component, or that a third component may be disposed therebetween.

Like reference numerals refer to like components throughout. Also, in the drawings, the thicknesses, ratios, and dimensions of the components are exaggerated for effective description of technical contents. The term “and/or” includes all of one or more combinations that can be defined by associated items.

It will be understood that, although the terms “first,” “second,” etc. may be utilized herein to describe one or more suitable components, these components should not be limited by these terms. These terms are only utilized to distinguish one component from another. For example, a first component could be termed a second component, and, similarly, a second component could be termed a first component, without departing from the scope of example embodiments of the present disclosure.

As utilized herein, the singular forms, “a,” “an,” and “the” are intended to include the plural forms as well, unless the context clearly indicates otherwise.

In some embodiments, terms such as “below,” “under,” “on,” and “above” may be utilized to describe the relationship between components illustrated in the drawings. The terms are utilized as a relative concept and are described with reference to the direction indicated in the drawings.

It should be understood that the terms “comprise,” or “have” are intended to specify the presence of stated features, integers, steps, operations, components, parts, or combinations thereof in the disclosure, but do not preclude the presence or addition of one or more other features, integers, steps, operations, components, parts, or combinations thereof.

As utilized herein, the term “and/or” includes any and all combinations of one or more of the associated listed items. Throughout the disclosure, the expression “at least one of a, b or c”, “at least one of a-c”, “at least one of a to c”, “at least one of a, b, and/or c”, “at least one among a to c”, etc., indicates only a, only b, only c, both (e.g., simultaneously) a and b, both (e.g., simultaneously) a and c, both (e.g., simultaneously) b and c, all of a, b, and c, or variations thereof.

In the present specification, “including A or B”, “A and/or B”, etc., represents A or B, or A and B.

As used herein, the term “substantially,” “about,” and similar terms are used as terms of approximation and not as terms of degree, and are intended to account for the inherent deviations in measured or calculated values that would be recognized by those of ordinary skill in the art. “About” or “substantially”, as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” or “substantially” may mean within one or more standard deviations, or within ±30%, 20%, 10%, 5% of the stated value.

Depending on context, a divalent group may refer or be a polyvalent group (e.g., trivalent, tetravalent, etc., and not just divalent) per, e.g., the structure of a formula in connection with which of the terms are utilized.

The light emitting element and/or any other relevant devices or components according to embodiments of the present invention described herein may be implemented utilizing any suitable hardware, firmware (e.g. an application-specific integrated circuit), software, or a combination of software, firmware, and hardware. For example, the various components of the device may be formed on one integrated circuit (IC) chip or on separate IC chips. Further, the various components of the [device] may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the exemplary embodiments of the present invention.

Unless otherwise defined, all terms (including technical and scientific terms) utilized herein have the same meaning as commonly understood by one of ordinary skill in the art to which the present disclosure belongs. In some embodiments, it will be understood that terms, such as those defined in commonly utilized dictionaries, should be interpreted as having a meaning that is consistent with their meaning in the context of the relevant art and will not be interpreted in an idealized or overly formal sense unless expressly so defined herein.

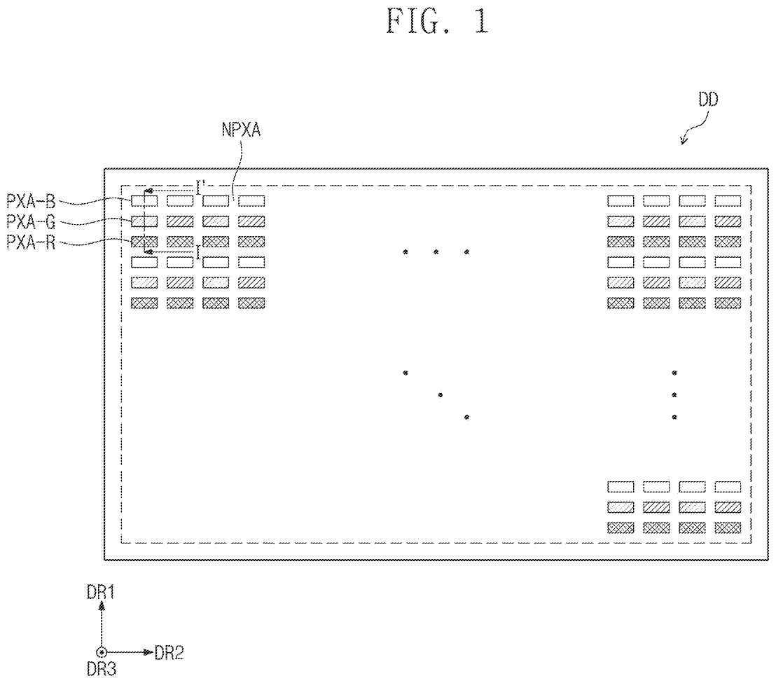

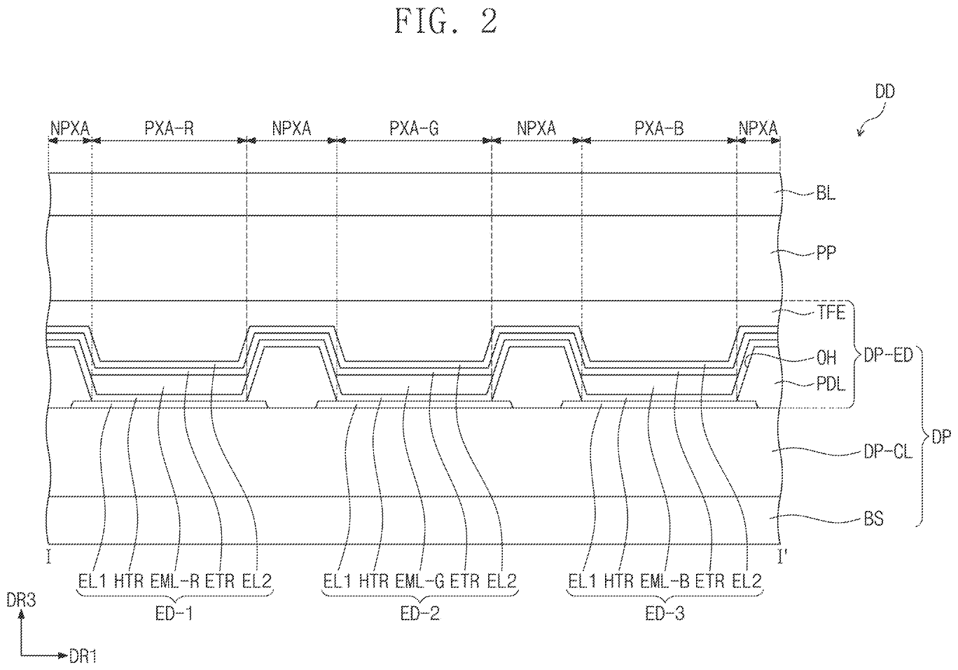

Hereinafter, embodiments of the present disclosure will be described with reference to the accompanying drawings. FIG. 1 is a plan view illustrating an embodiment of a display device DD. FIG. 2 is a cross-sectional view of the display device DD of the embodiment. FIG. 2 is a cross-sectional view illustrating a part taken along line I-I′ of FIG. 1.

The display device DD may include a display panel DP and an optical layer PP disposed on the display panel DP. The display panel DP may include light emitting elements ED-1, ED-2, and ED-3. The display device DD may include a plurality of light emitting elements ED-1, ED-2, and ED-3. The optical layer PP may be disposed on the display panel DP to control reflected light in the display panel DP due to external light. The optical layer PP may include, for example, a polarization layer or a color filter layer. In some embodiments, unlike the configuration illustrated in the drawing, the optical layer PP may not be provided from the display device DD of an embodiment.

A base substrate BL may be disposed on the optical layer PP. The base substrate BL may be a member which provides a base surface on which the optical layer PP disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, unlike the configuration illustrated, in an embodiment, the base substrate BL may not be provided.

The display device DD according to an embodiment may further include a filling layer. The filling layer may be disposed between a display element layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display element layer DP-ED. The display element layer DP-ED may include a pixel defining film PDL, the light emitting elements ED-1, ED-2, and ED-3 disposed between portions of the pixel defining film PDL, and an encapsulation layer TFE disposed on the light emitting elements ED-1, ED-2, and ED-3.

The base layer BS may be a member which provides a base surface on which the display element layer DP-ED is disposed. The base layer BS may be a glass substrate, a metal substrate, a plastic substrate, etc. However, the embodiment is not limited thereto, and the base layer BS may be an inorganic layer, an organic layer, or a composite material layer.

In an embodiment, the circuit layer DP-CL is disposed on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display element layer DP-ED.

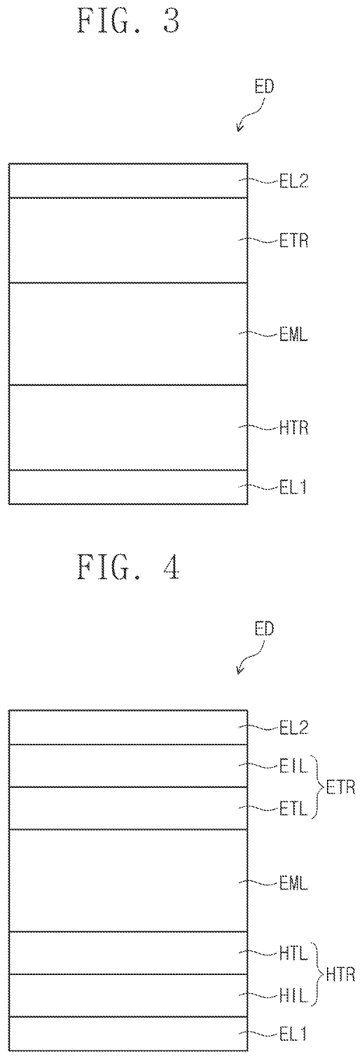

Each of the light emitting elements ED-1, ED-2, and ED-3 may have a structure of each light emitting element ED of embodiments according to FIGS. 3 to 6, which will be described later. Each of the light emitting elements ED-1, ED-2, and ED-3 may include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light emitting elements ED-1, ED-2, and ED-3 are disposed in openings OH defined in the pixel defining film PDL, and the hole transport region HTR, the electron transport region ETR, and the second electrode EL2 are provided as a common layer in the entire light emitting elements ED-1, ED-2, and ED-3. However, the embodiment of the present disclosure is not limited thereto, and unlike the configuration illustrated in FIG. 2, the hole transport region HTR and the electron transport region ETR in an embodiment may be provided by being patterned inside the openings OH defined in the pixel defining film PDL. For example, the hole transport region HTR, the emission layers EML-R, EML-G, and EML-B, and the electron transport region ETR of the light emitting elements ED-1, ED-2, and ED-3 in an embodiment may be provided by being patterned in an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be formed by laminating one layer or a plurality of layers. The encapsulation layer TFE may include at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film may protect the display element layer DP-ED from moisture/oxygen, and the encapsulation-organic film may protect the display element layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not particularly limited thereto. The encapsulation-organic film may include an acrylic-based compound, an epoxy-based compound, and/or the like. The encapsulation-organic film may include a photopolymerizable organic material, but the embodiment of the present disclosure is not particularly limited thereto. The encapsulation layer TFE may be disposed on the second electrode EL2 and may be disposed filling the opening OH.

Referring to FIGS. 1 and 2, the display device DD may include a non-light emitting region NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced apart from each other on a plane.

Each of the light emitting regions PXA-R, PXA-G, and PXA-B may be a region divided by the pixel defining film PDL. The non-light emitting areas NPXA may be areas between the adjacent light emitting areas PXA-R, PXA-G, and PXA-B, which correspond to the pixel defining film PDL. In some embodiments, in the specification, the light emitting regions PXA-R, PXA-G, and PXA-B may respectively correspond to pixels. The pixel defining film PDL may divide the light emitting elements ED-1, ED-2, and ED-3. The emission layers EML-R, EML-G, and EML-B of the light emitting elements ED-1, ED-2, and ED-3 may be disposed in openings OH defined in the pixel defining film PDL and separated from each other.

The light emitting regions PXA-R, PXA-G, and PXA-B may be divided into a plurality of groups according to the color of light generated from the light emitting elements ED-1, ED-2, and ED-3. In the display device DD of an embodiment illustrated in FIGS. 1 and 2, three light emitting regions PXA-R, PXA-G, and PXA-B, which emit red light, green light, and blue light, respectively, are exemplarily illustrated. For example, the display device DD of an embodiment may include the red light emitting region PXA-R, the green light emitting region PXA-G, and the blue light emitting region PXA-B that are separated from each other.

In the display device DD according to an embodiment, the plurality of light emitting elements ED-1, ED-2 and ED-3 may be configured to emit light beams having wavelengths different from each other. For example, in an embodiment, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2, and ED-3 may be configured to emit light beams in substantially the same wavelength range or at least one light emitting element may be configured to emit a light beam in a wavelength range different from the others. For example, the first to third light emitting elements ED-1, ED-2, and ED-3 may all emit blue light.

The light emitting regions PXA-R, PXA-G, and PXA-B in the display device DD according to an embodiment may be arranged in a stripe form. Referring to FIG. 1, the plurality of red light emitting regions PXA-R, the plurality of green light emitting regions PXA-G, and the plurality of blue light emitting regions PXA-B each may be arranged along a second directional axis DR2. In some embodiments, the red light emitting region PXA-R, the green light emitting region PXA-G, and the blue light emitting region PXA-B may be alternately arranged in this order along a first directional axis DR1.

FIGS. 1 and 2 illustrate that all the light emitting regions PXA-R, PXA-G, and PXA-B have similar area, but the embodiment of the present disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas when viewed on a plane defined by the first directional axis DR1 and the second directional axis DR2.

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display device DD. For example, the arrangement form of the light emitting regions PXA-R, PXA-G, and PXA-B may be a pentile (PENTILE®) arrangement form or a diamond (Diamond Pixel™) arrangement form.

In some embodiments, the areas of the light emitting regions PXA-R, PXA-G, and PXA-B may be different from each other. For example, in an embodiment, the area of the green light emitting region PXA-G may be smaller than that of the blue light emitting region PXA-B, but the embodiment of the present disclosure is not limited thereto.

Hereinafter, FIGS. 3 to 6 are cross-sectional views schematically illustrating light emitting elements according to embodiments. Each of the light emitting elements ED according to embodiments may include a first electrode EL1, a hole transport region HTR, an emission layer EML, an electron transport region ETR, and a second electrode EL2 that are sequentially stacked.

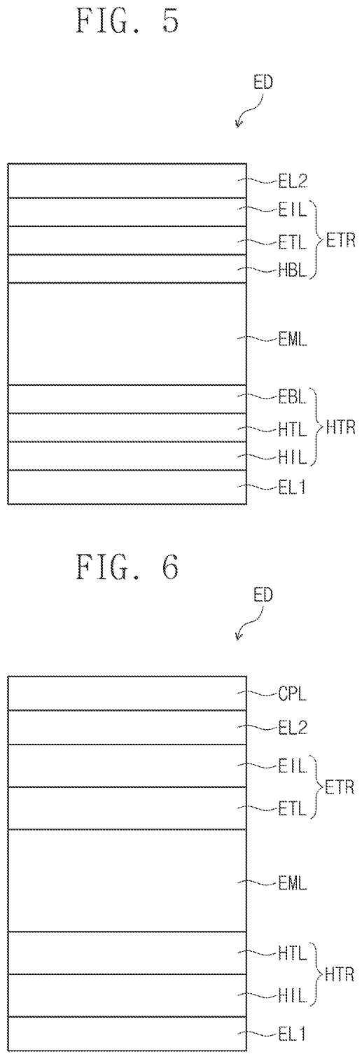

Compared with FIG. 3, FIG. 4 illustrates a cross-sectional view of a light emitting element ED of an embodiment, in which a hole transport region HTR includes a hole injection layer HIL and a hole transport layer HTL, and an electron transport region ETR includes an electron injection layer EIL and an electron transport layer ETL. In some embodiments, compared with FIG. 3, FIG. 5 illustrates a cross-sectional view of a light emitting element ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting element ED of an embodiment including a capping layer CPL disposed on a second electrode EL2.

In the light emitting element ED of an embodiment, the emission layer EML may include a nitrogen-containing compound of an embodiment. The nitrogen-containing compound of an embodiment may be one in which three carbazole groups are directly or indirectly bonded to a hexagonal monocyclic ring containing at least one N as a ring-forming atom. More specifically, two carbazole groups among the three carbazole groups may be directly bonded to a hexagonal monocyclic ring containing N as a ring-forming atom, and the remaining one carbazole group may be indirectly bonded to the hexagonal monocyclic ring containing N as a ring-forming atom via a biphenyl group. For example, the hexagonal monocyclic ring containing N as a ring-forming atom may be a triazine group. In the specification, the nitrogen-containing compound may be the same as the first compound.

In the specification, the term “substituted or unsubstituted” may refer to that a group is unsubstituted or substituted with at least one substituent selected from the group consisting of a deuterium atom, a halogen atom, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified above may be substituted or unsubstituted. For example, a biphenyl group may be interpreted as an aryl group or a phenyl group substituted with a phenyl group.

In the specification, the phrase “bonded to an adjacent group to form a ring” may refer to that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be a linear, branched or cyclic type or kind. The number of carbons in the alkyl group is 1 to 60, 1 to 50, 1 to 30, 1 to 20, 1 to 10, or 1 to 6. Examples of the alkyl group may include a methyl group, an ethyl group, an n-propyl group, an isopropyl group, a cyclopropyl group, an n-butyl group, an s-butyl group, a t-butyl group, an i-butyl group, a 2-ethylbutyl group, a cyclobutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a cycloheptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, a cyclononyl group, a norbornyl group, an n-decyl group, a cyclodecyl group, a 1-adamantyl group, a 2-adamantyl group, an isobornyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, a bicycloheptyl group, an n-triacontyl group, etc., but the embodiment of the present disclosure is not limited thereto.

In the specification, an alkenyl group refers to a hydrocarbon group including at least one carbon double bond in the middle or terminal of an alkyl group having 2 or more carbon atoms. The alkenyl group may be linear or branched. The number of carbon atoms in the alkenyl group is not specifically limited, but 2 to 60, 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, etc., but the embodiment of the present disclosure is not limited thereto.

In the specification, an alkynyl group refers to a hydrocarbon group including at least one carbon triple bond in the middle or terminal of an alkyl group having 2 or more carbon atoms. The alkynyl group may be linear or branched. The number of carbon atoms in the alkynyl group is not specifically limited, but 2 to 60, 2 to 30, 2 to 20, or 2 to 10. Specific examples of the alkynyl group may include an ethynyl group, a propynyl group, etc., but the embodiment of the present disclosure is not limited thereto.

In the specification, the hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 60, 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but the embodiment of the present disclosure is not limited thereto.

In the specification, the heterocyclic group refers to any functional group or substituent derived from a ring containing at least one of B, O, N, P, Si, or S as a heteroatom. The heterocyclic group includes an aliphatic heterocyclic group and an aromatic heterocyclic group. The aromatic heterocyclic group may be a heteroaryl group. The aliphatic heterocyclic group and the aromatic heterocyclic group may be monocyclic or polycyclic.

In the specification, the heterocyclic group may contain at least one of B, O, N, P, Si or S as a heteroatom. When the heterocyclic group contains two or more heteroatoms, the two or more heteroatoms may be the same as or different from each other. The heterocyclic group may be a monocyclic heterocyclic group or a polycyclic heterocyclic group and has the concept including a heteroaryl group. The number of ring-forming carbon atoms in the heterocyclic group may be 2 to 30, 2 to 20, or 2 to 10.

In the specification, the aliphatic heterocyclic group may include at least one of B, O, N, P, Si or S as a heteroatom. The number of ring-forming carbon atoms in the aliphatic heterocyclic group may be 2 to 60, 2 to 30, 2 to 20, or 2 to 10. Examples of the aliphatic heterocyclic group include an oxirane group, a tyran group, a pyrrolidine group, a piperidine group, a tetrahydrofuran group, a tetrahydrothiophene group, a thian group, a tetrahydropyran group, a 1,4-dioxane group, etc., but the embodiment of the present disclosure is not limited thereto.

In the specification, the heteroaryl group may contain at least one of B, O, N, P, Si, or S as a heteroatom. When the heteroaryl group contains two or more heteroatoms, the two or more heteroatoms may be the same as or different from each other. The heteroaryl group may be a monocyclic heterocyclic group or a polycyclic heterocyclic group. The ring-forming carbon number of the heteroaryl group may be 2 to 60, 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but the embodiment of the present disclosure is not limited thereto.

In the specification, the above description of the aryl group may be applied to an arylene group except that the arylene group is a divalent group. The above description of the heteroaryl group may be applied to a heteroarylene group except that the heteroarylene group is a divalent group.

In the specification, a silyl group may refer to that a silicon atom is bonded to the alkyl group or the aryl group as defined above. The silyl group includes an alkylsilyl group and an arylsilyl group. Examples of the silyl group may include a trimethylsilyl group, a triethylsilyl group, a t-butyldimethylsilyl group, an ethyldimethylsilyl group, a propyldimethylsilyl group, a triphenylsilyl group, a diphenylsilyl group, a phenylsilyl group, etc., but the embodiment of the present disclosure is not limited thereto.

In the specification, the number of carbon atoms in the sulfinyl group and the sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the specification, the thio group may include an alkylthio group and an arylthio group. The thio group may refer to that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the embodiment of the present disclosure is not limited thereto.

In the specification, an oxy group may refer to that an oxygen atom is bonded to the alkyl group or the aryl group as defined above. The oxy group may include an alkoxy group and an aryl oxy group. The alkoxy group may be a linear chain, a branched chain or a ring chain. The number of carbon atoms in the alkoxy group is not specifically limited, but may be, for example, 1 to 30, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc., but the embodiment of the present disclosure is not limited thereto.

In the specification, a boryl group may refer to that a boron atom is bonded to the alkyl group or the aryl group as defined above. The boron group includes an alkyl boron group and an aryl boron group. Examples of the boron group may include a dimethylboron group, a diethylboron group, a t-butylmethylboron group, a diphenylboron group, a phenylboron group, etc., but the embodiment of the present disclosure is not limited thereto.

In the specification, the number of carbon atoms in an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, etc., but the embodiment of the present disclosure is not limited thereto.

In the specification, the alkyl group in an alkylthio group, an alkylsulfinyl group, an alkylsulfonyl group, an alkoxy group, an alkyl amine group, an alkyl boron group, an alkyl silyl group, and an alkyl amine group is the same as the examples of the alkyl group described above.

In the specification, the aryl group in an aryloxy group, an arylthio group, an arylsulfinyl group, an arylsulfonyl group, an arylamine group, an arylboron group, and an arylsilyl group may be the same as the examples of the aryl group described above.

In the specification, a direct linkage may refer to a single bond. In the specification,

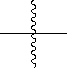

and “-*” refer to a position to be linked.

The nitrogen-containing compound of an embodiment may be represented by Formula 1. In an embodiment, the emission layer EML may include the nitrogen-containing compound represented by Formula 1:

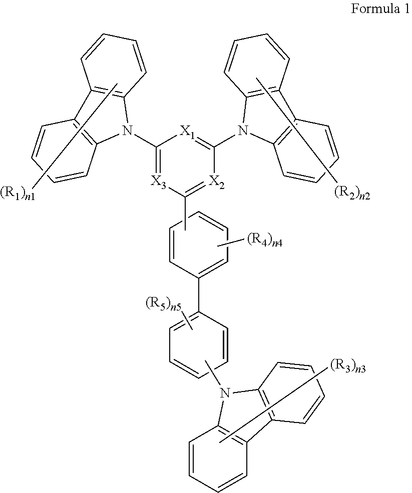

In Formula 1, at least one among X₁ to X₃ may be N, and the rest may each independently be CR₆. In an embodiment, each of X₁ to X₃ may be N. In Formula 1, when all of X₁ to X₃ are N, the nitrogen-containing compound of an embodiment may include triazine as a central structure.

In some embodiments, n1 to n3 may each independently be an integer of 0 to 8. When n1 is an integer of 2 or greater, a plurality of R₁'s may be the same as each other or at least one may be different from the others. When n2 is an integer of 2 or greater, a plurality of R₂'s may be the same as each other or at least one may be different from the others. When n3 is an integer of 2 or greater, a plurality of R₃'s may be the same as each other or at least one may be different from the others.

n4 and n5 may each independently be an integer of 0 to 4. When n4 is an integer of 2 or greater, a plurality of R₄'s may be the same as each other or at least one may be different from the others. When n5 is an integer of 2 or greater, a plurality of R₅'s may be the same as each other or at least one may be different from the others.

In Formula 1, R₁ to R₆ may each independently be a hydrogen atom, a deuterium atom, a halogen atom, a hydroxy group, a cyano group, a nitro group, a substituted or unsubstituted amine group, a substituted or unsubstituted oxy group, a substituted or unsubstituted thio group, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted aryl group having 6 to 60 ring-forming carbon atoms, or a substituted or unsubstituted heteroaryl group having 2 to 60 ring-forming carbon atoms.

The nitrogen-containing compound of an embodiment may include a deuterium atom as a substituent. In an embodiment, at least one among R₁ to R₅ may be a deuterium atom, or may include a substituent containing a deuterium atom. For example, at least one among R₁ to R₃ may be a deuterium atom. At least one of R₄ or R₅ may be a deuterium atom, or at least one of R₄ or R₅ may be an aryl group substituted with a deuterium atom, or a heteroaryl group substituted with a deuterium atom. However, this is an example, and the embodiment of the present disclosure is not limited thereto.

More specifically, n1 and n2 may be 8, and a plurality of R₁'s and R₂'s may be deuterium atoms. n3 may be 2 or 8, and a plurality of R₃'s may each independently be deuterium atoms or unsubstituted t-butyl groups. n4 may be 1, and R₄ may be a substituted or unsubstituted phenyl group, a substituted or unsubstituted carbazole group, an unsubstituted dibenzofuran group, or an unsubstituted dibenzothiophene group. In some embodiments, n5 may be 1, and R₅ may be a substituted or unsubstituted phenyl group, a substituted or unsubstituted carbazole group, an unsubstituted dibenzofuran group, or an unsubstituted dibenzothiophene group.

For example, R₁ to R₆ may each independently be a hydrogen atom, a deuterium atom, an unsubstituted t-butyl group, a substituted or unsubstituted phenyl group, a substituted or unsubstituted carbazole group, an unsubstituted dibenzofuran group, or an unsubstituted dibenzothiophene group. In an embodiment, R₁ to R₆ may each independently be a hydrogen atom, a deuterium atom, or represented by any one among R-1 to R-7:

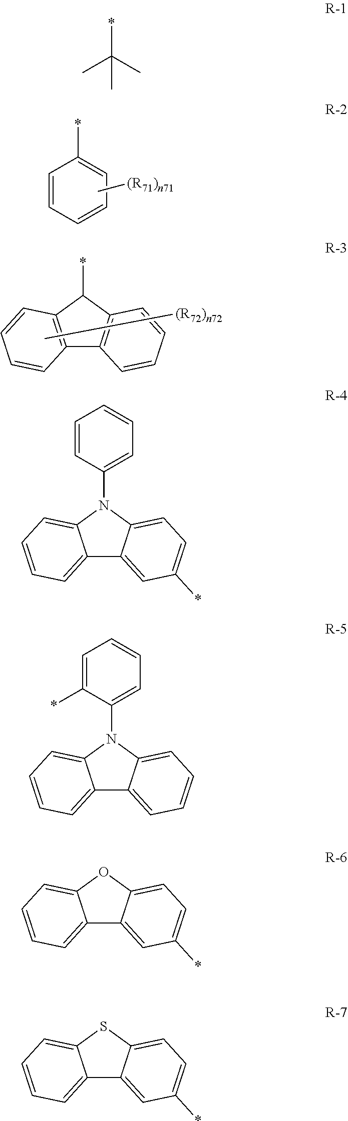

In R-2 and R-3, n71 may be an integer of 0 to 5, and n72 may be an integer of 0 to 8. When n71 is an integer of 2 or greater, a plurality of R₇₁'s may be the same as each other or at least one may be different from the others. When n72 is an integer of 2 or greater, a plurality of R₇₂'s may be the same as each other or at least one may be different from the others. In R-2 and R-3, R₇₁ and R₇₂ may each independently be a hydrogen atom, a deuterium atom, or an unsubstituted t-butyl group. For example, R-2 may be represented by R-21 or R-22, and R-3 may be represented by any one among R-31 to R-34:

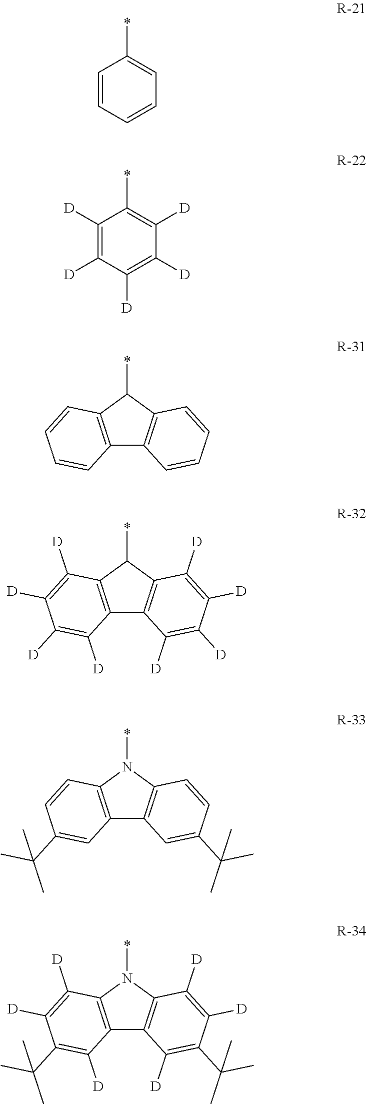

In an embodiment, Formula 1 may be represented by Formula 1-1 or Formula 1-2: Formula 1-1 and Formula 1-2 represent the cases where the bonding position of the phenyl group including R₅ is specified in the phenyl group including R₄ in Formula 1.

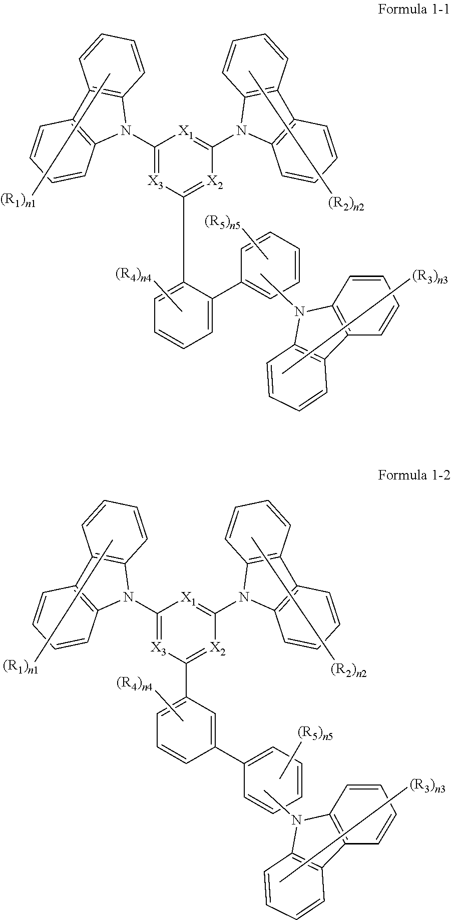

Formula 1-1 represents the case where the ring group containing X₁ to X₃ and the phenyl group including R₅ are bonded at the ortho-position in the phenyl group including R₄ in Formula 1. Formula 1-2 represents the case where the ring group containing X₁ to X₃ and the phenyl group including R₅ are bonded at the meta-position in the phenyl group including R₄ in Formula 1. In Formula 1-1 and Formula 1-2, the same as described in Formula 1 may be applied to X₁ to X₃, n1 to n5, and R₁ to R₅.

In an embodiment, Formula 1-1 may be represented by Formula 1-1A or Formula 1-1B. Formula 1-1A and Formula 1-1B represent the cases where the bonding position of the carbazole group including R₃ is specified in the phenyl group including R₅ in Formula 1-1.

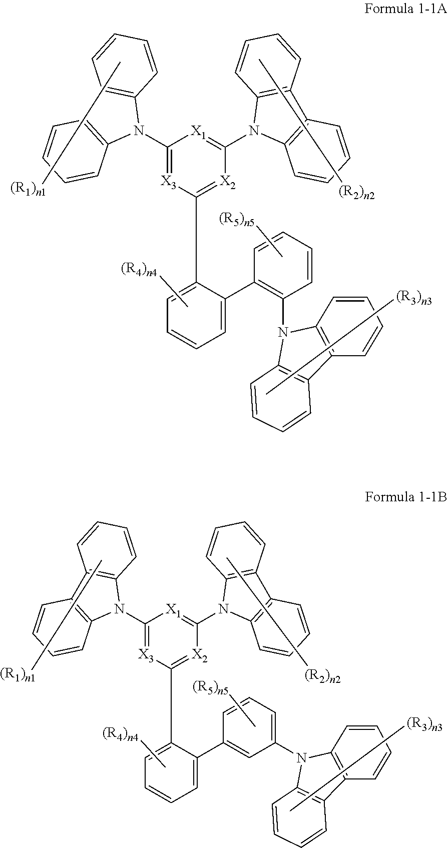

Formula 1-1A represents the case where the phenyl group including R₄ and the carbazole group including R₃ are bonded at the ortho-position in the phenyl group including R₅ in Formula 1-1. Formula 1-1B represents the case where the phenyl group including R₄ and the carbazole group including R₃ are bonded at the meta-position in the phenyl group including R₅ in Formula 1-1. In Formula 1-1A and Formula 1-1B, the same as described in Formula 1-1 may be applied to X₁ to X₃, n1 to n5, and R₁ to R₅.

In an embodiment, Formula 1-2 may be represented by Formula 1-2A or Formula 1-2B. Formula 1-2A and Formula 1-2B represent the cases where the bonding position of the carbazole group including R₃ is specified in the phenyl group including R₅ in Formula 1-2.

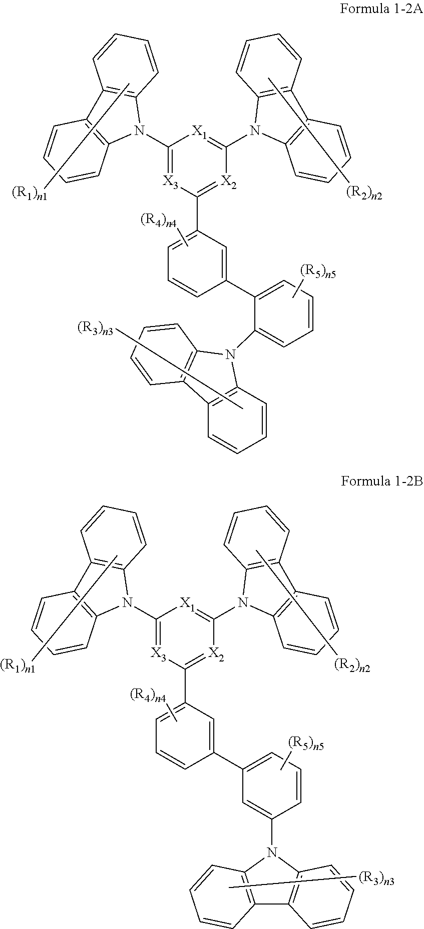

Formula 1-2A represents the case where the phenyl group including R₄ and the carbazole group including R₃ are bonded at the ortho-position in the phenyl group including R₅ in Formula 1-2. Formula 1-2B represents the case where the phenyl group including R₄ and the carbazole group including R₃ are bonded at the meta-position in the phenyl group including R₅ in Formula 1-2. In Formula 1-2A and Formula 1-2B, the same as described in Formula 1-2 may be applied to X₁ to X₃, n1 to n5, and R₁ to R₅.

In an embodiment, Formula 1 may be represented by any one among Formula 1-3 to Formula 1-5. Formula 1-3 to Formula 1-5 represent the cases where the bonding position of R₅ and the bonding position of the carbazole group including R₃ are specified when n5 is 1 in Formula 1. In Formula 1-3 to Formula 1-5, R₅₁ corresponds to R₅ in the case where n5 is 1 in Formula 1.

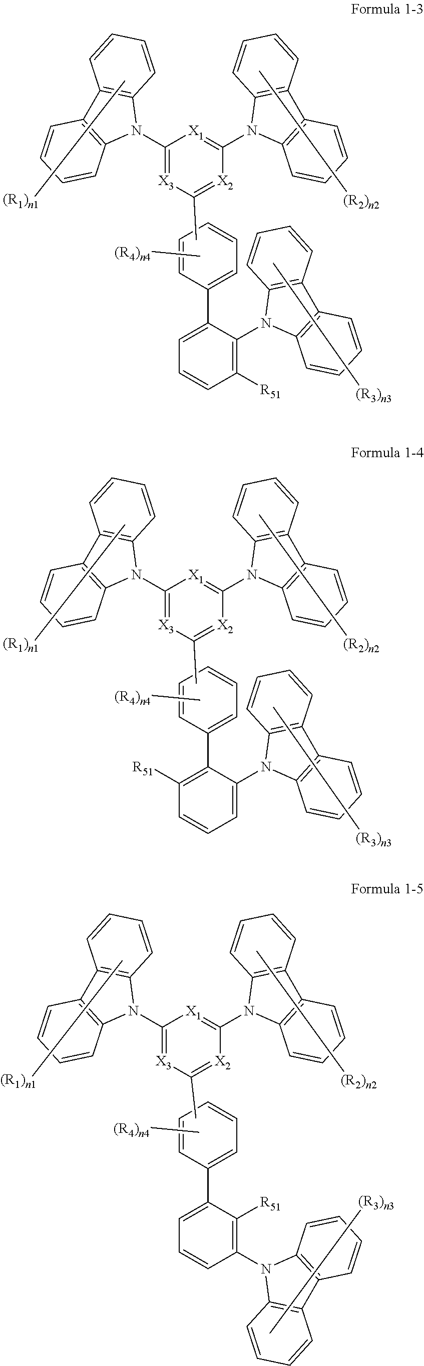

Formula 1-3 represents the case where R₅₁ is bonded at the meta-position with respect to the phenyl group including R₄ and the carbazole group including R₃ is bonded at the ortho-position. Formula 1-4 represents the case where R₅₁ is bonded at the ortho-position with respect to the phenyl group including R₄ and the carbazole group including R₃ is bonded at the other ortho-position. Formula 1-5 represents the case where R₅₁ is bonded at the ortho-position with respect to the phenyl group including R₄ and the carbazole group including R₃ is bonded at the meta-position.

In Formula 1-3 to Formula 1-5, R₅₁ may be a substituted or unsubstituted phenyl group, a substituted or unsubstituted carbazole group, a substituted or unsubstituted dibenzofuran group, or a substituted or unsubstituted dibenzothiophene group. In Formula 1-3 to Formula 1-5, the same as described in Formula 1 may be applied to X₁ to X₃, n1 to n4, and R₁ to R₄.

The nitrogen-containing compound of an embodiment may be represented by any one among compounds in Compound Group 1. The light emitting element ED of an embodiment may include any one among the compounds of Compound Group 1. In Compound Group 1, D is a deuterium atom.

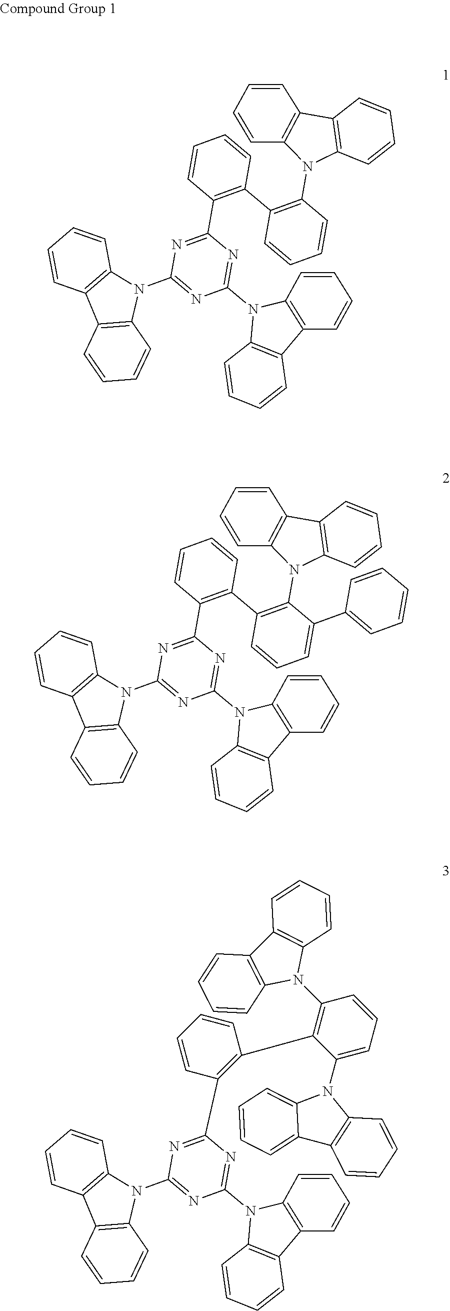 [SMILES: c1ccc(-c2ccccc2-n2c3ccccc3c3ccccc32)c(-c2nc(-n3c4ccccc4c4ccccc43)nc(-n3c4ccccc4c4ccccc43)n2)c1 ; c1ccc(-c2cccc(-c3ccccc3-c3nc(-n4c5ccccc5c5ccccc54)nc(-n4c5ccccc5c5ccccc54)n3)c2-n2c3ccccc3c3ccccc32)cc1 ; c1ccc(-c2c(-n3c4ccccc4c4ccccc43)cccc2-n2c3ccccc3c3ccccc32)c(-c2nc(-n3c4ccccc4c4ccccc43)nc(-n3c4ccccc4c4ccccc43)n2)c1]

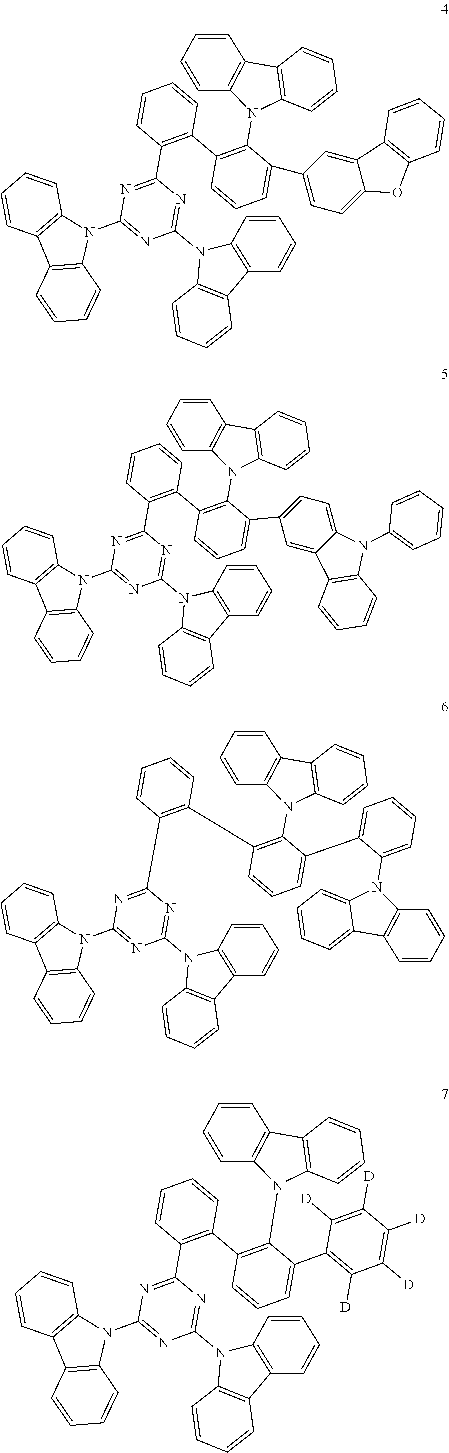

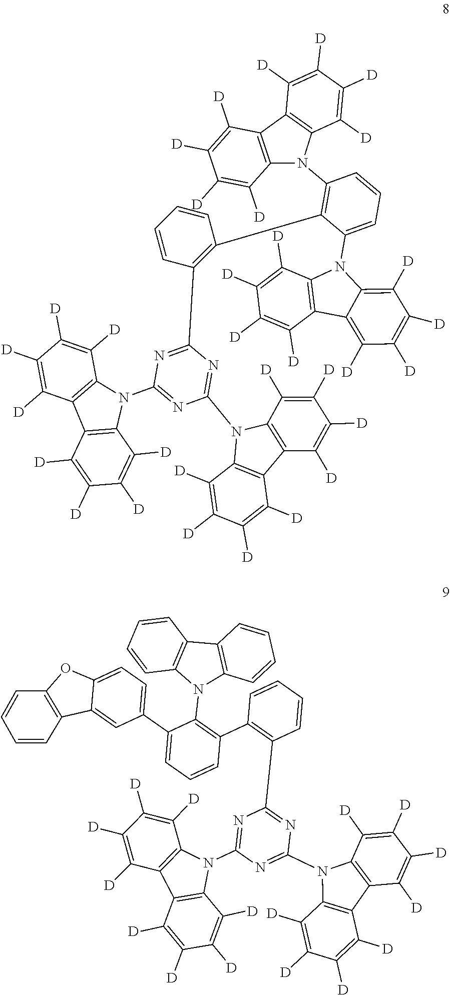

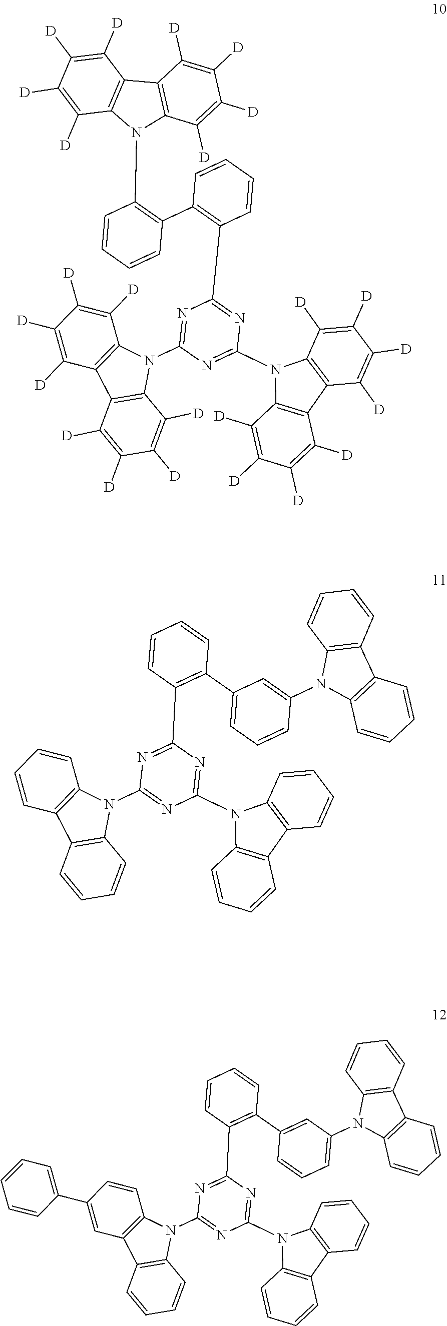 [SMILES: [2H]c1c([2H])c([2H])c2c(c1[2H])c1c([2H])c([2H])c([2H])c([2H])c1n2-c1nc(-c2ccccc2-c2ccccc2-n2c3c([2H])c([2H])c([2H])c([2H])c3c3c([2H])c([2H])c([2H])c([2H])c32)nc(-n2c3c([2H])c([2H])c([2H])c([2H])c3c3c([2H])c([2H])c([2H])c([2H])c32)n1 ; c1cc(-c2ccccc2-c2nc(-n3c4ccccc4c4ccccc43)nc(-n3c4ccccc4c4ccccc43)n2)cc(-n2c3ccccc3c3ccccc32)c1 ; c1ccc(-c2ccc3c(c2)c2ccccc2n3-c2nc(-c3ccccc3-c3cccc(-n4c5ccccc5c5ccccc54)c3)nc(-n3c4ccccc4c4ccccc43)n2)cc1]

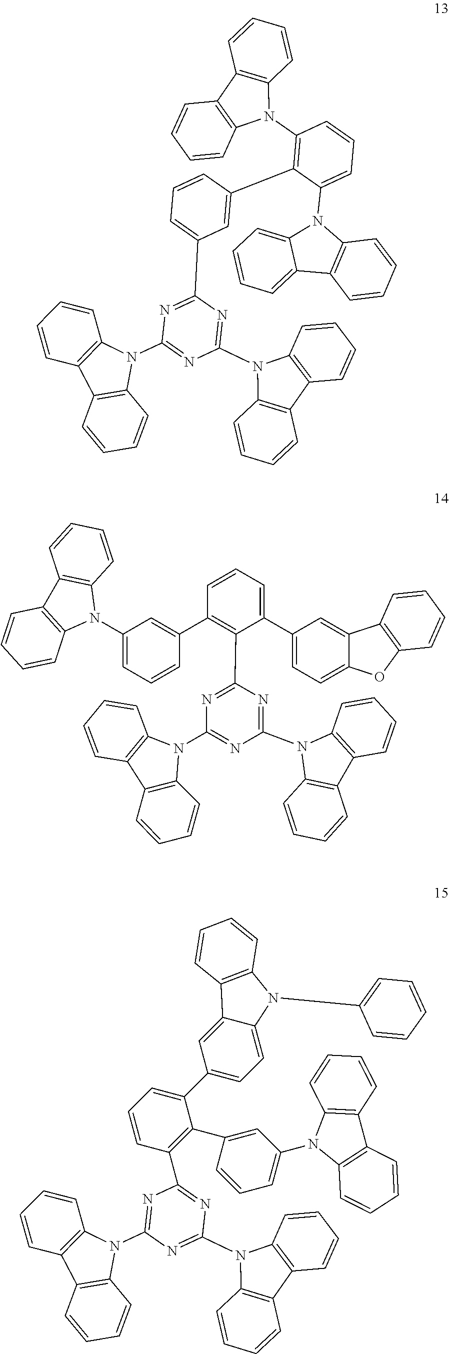 [SMILES: c1cc(-c2nc(-n3c4ccccc4c4ccccc43)nc(-n3c4ccccc4c4ccccc43)n2)cc(-c2c(-n3c4ccccc4c4ccccc43)cccc2-n2c3ccccc3c3ccccc32)c1 ; c1cc(-c2cccc(-c3ccc4oc5ccccc5c4c3)c2-c2nc(-n3c4ccccc4c4ccccc43)nc(-n3c4ccccc4c4ccccc43)n2)cc(-n2c3ccccc3c3ccccc32)c1 ; c1ccc(-n2c3ccccc3c3cc(-c4cccc(-c5nc(-n6c7ccccc7c7ccccc76)nc(-n6c7ccccc7c7ccccc76)n5)c4-c4cccc(-n5c6ccccc6c6ccccc65)c4)ccc32)cc1]

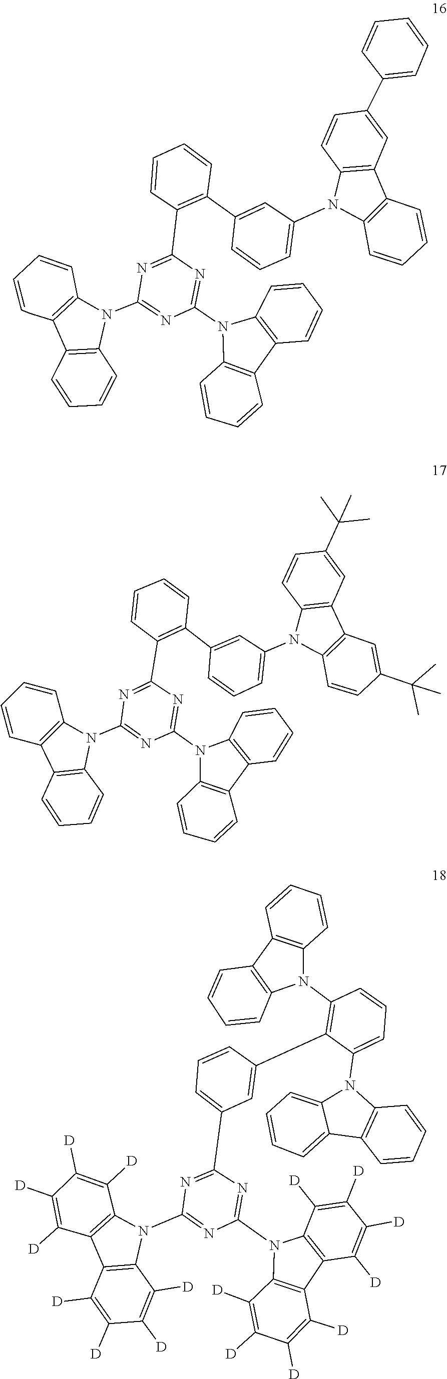 [SMILES: C1=CCc2c(n(-c3nc(-c4ccccc4-c4cccc(-n5c6ccccc6c6cc(-c7ccccc7)ccc65)c4)nc(-n4c5ccccc5c5ccccc54)n3)c3ccccc23)C=C1 ; CC(C)(C)C1=CCC2=C(C=C1)N(c1cccc(-c3ccccc3-c3nc(-n4c5ccccc5c5ccccc54)nc(-n4c5ccccc5c5ccccc54)n3)c1)Cc1ccc(C(C)(C)C)cc12 ; [2H]c1c([2H])c([2H])c2c(c1[2H])c1c([2H])c([2H])c([2H])c([2H])c1n2-c1nc(-c2cccc(-c3c(-n4c5ccccc5c5ccccc54)cccc3-n3c4ccccc4c4ccccc43)c2)nc(-n2c3c([2H])c([2H])c([2H])c([2H])c3c3c([2H])c([2H])c([2H])c([2H])c32)n1]

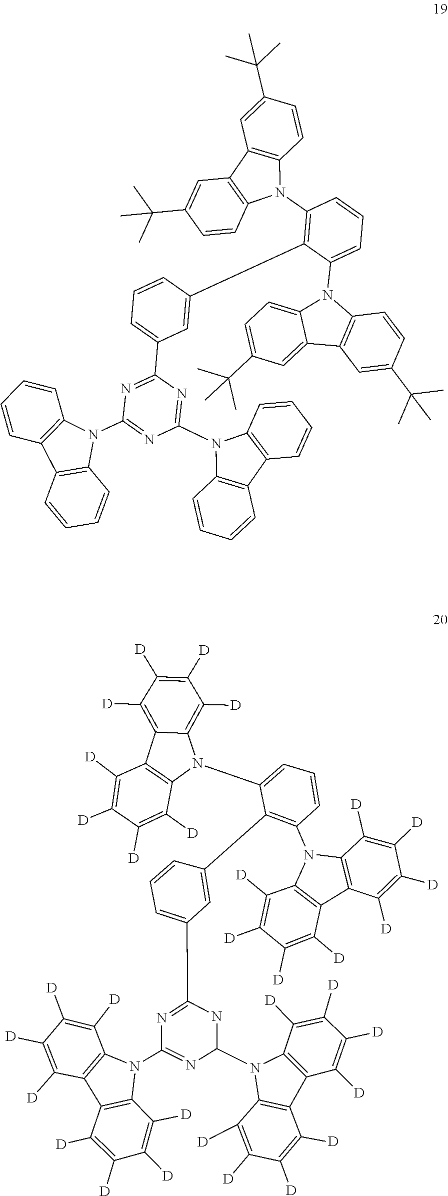

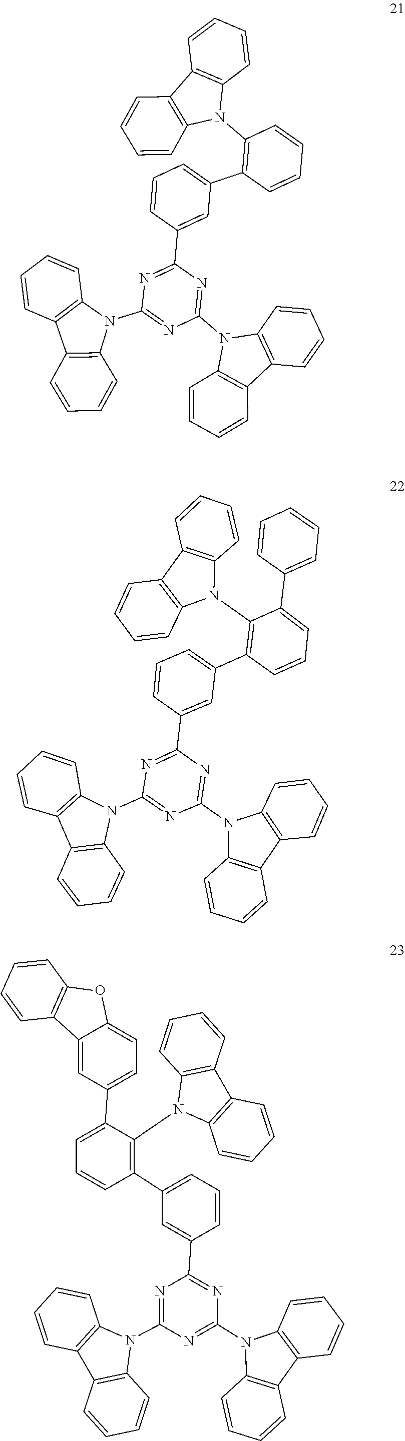 [SMILES: c1cc(-c2nc(-n3c4ccccc4c4ccccc43)nc(-n3c4ccccc4c4ccccc43)n2)cc(-c2ccccc2-n2c3ccccc3c3ccccc32)c1 ; C1=CCc2c(n(-c3nc(-c4cccc(-c5cccc(-c6ccccc6)c5-n5c6ccccc6c6ccccc65)c4)nc(-n4c5ccccc5c5ccccc54)n3)c3ccccc23)C=C1 ; c1cc(-c2nc(-n3c4ccccc4c4ccccc43)nc(-n3c4ccccc4c4ccccc43)n2)cc(-c2cccc(-c3ccc4oc5ccccc5c4c3)c2-n2c3ccccc3c3ccccc32)c1]

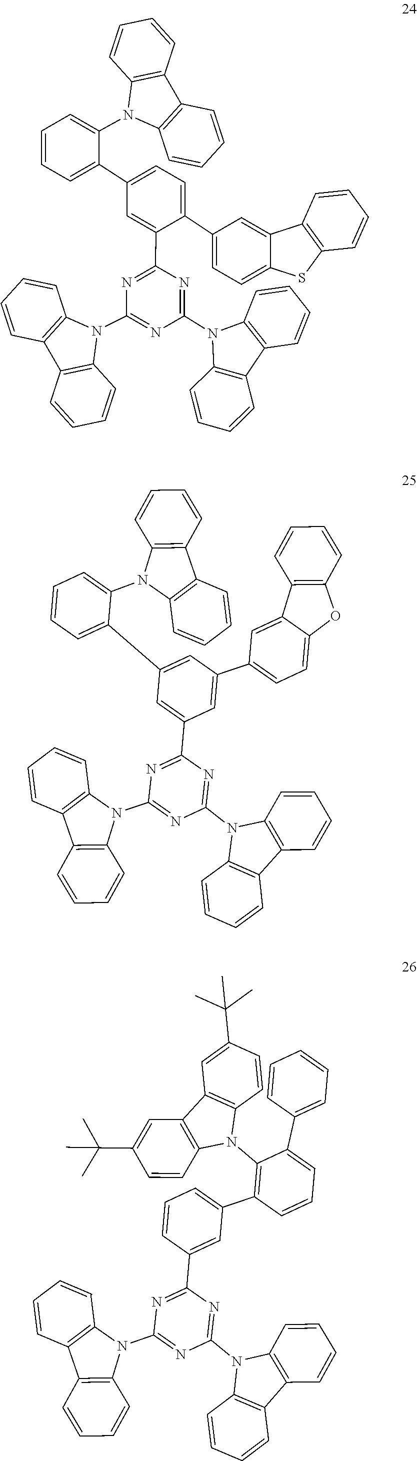 [SMILES: c1ccc(-n2c3ccccc3c3ccccc32)c(-c2ccc(-c3ccc4sc5ccccc5c4c3)c(-c3nc(-n4c5ccccc5c5ccccc54)nc(-n4c5ccccc5c5ccccc54)n3)c2)c1 ; c1ccc(-n2c3ccccc3c3ccccc32)c(-c2cc(-c3ccc4oc5ccccc5c4c3)cc(-c3nc(-n4c5ccccc5c5ccccc54)nc(-n4c5ccccc5c5ccccc54)n3)c2)c1 ; Cc1cccc2c3ccccc3n(-c3nc(-c4cccc(-c5cccc(-c6ccccc6)c5-n5c6ccc(C(C)(C)C)cc6c6cc(C(C)(C)C)ccc65)c4)nc(-n4c5ccccc5c5ccccc54)n3)c12]

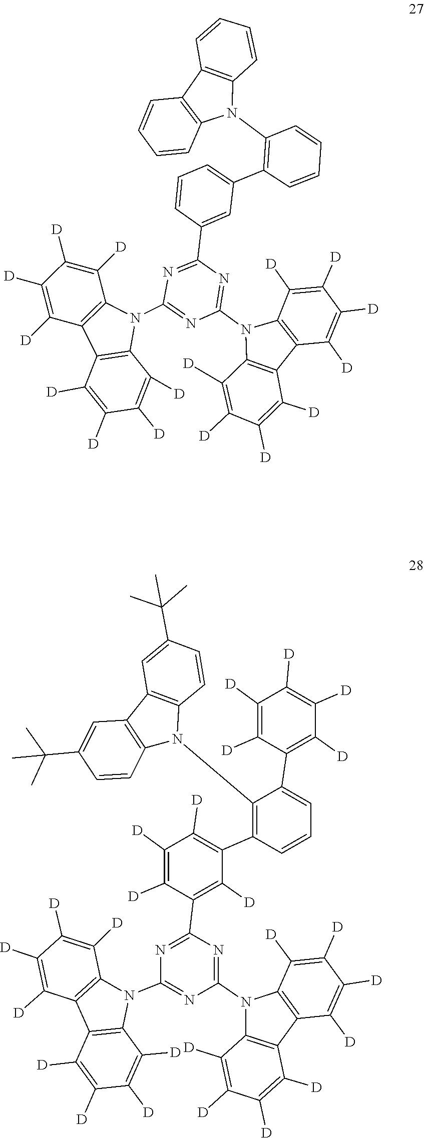 [SMILES: [2H]c1c([2H])c([2H])c2c(c1[2H])c1c([2H])c([2H])c([2H])c([2H])c1n2-c1nc(-c2cccc(-c3ccccc3-n3c4ccccc4c4ccccc43)c2)nc(-n2c3c([2H])c([2H])c([2H])c([2H])c3c3c([2H])c([2H])c([2H])c([2H])c32)n1 ; [2H]c1c([2H])c([2H])c(-c2cccc(-c3c([2H])c([2H])c([2H])c(-c4nc(-n5c6c([2H])c([2H])c([2H])c([2H])c6c6c([2H])c([2H])c([2H])c([2H])c65)nc(-n5c6c([2H])c([2H])c([2H])c([2H])c6c6c([2H])c([2H])c([2H])c([2H])c65)n4)c3[2H])c2-n2c3ccc(C(C)(C)C)cc3c3cc(C(C)(C)C)ccc32)c([2H])c1[2H]]

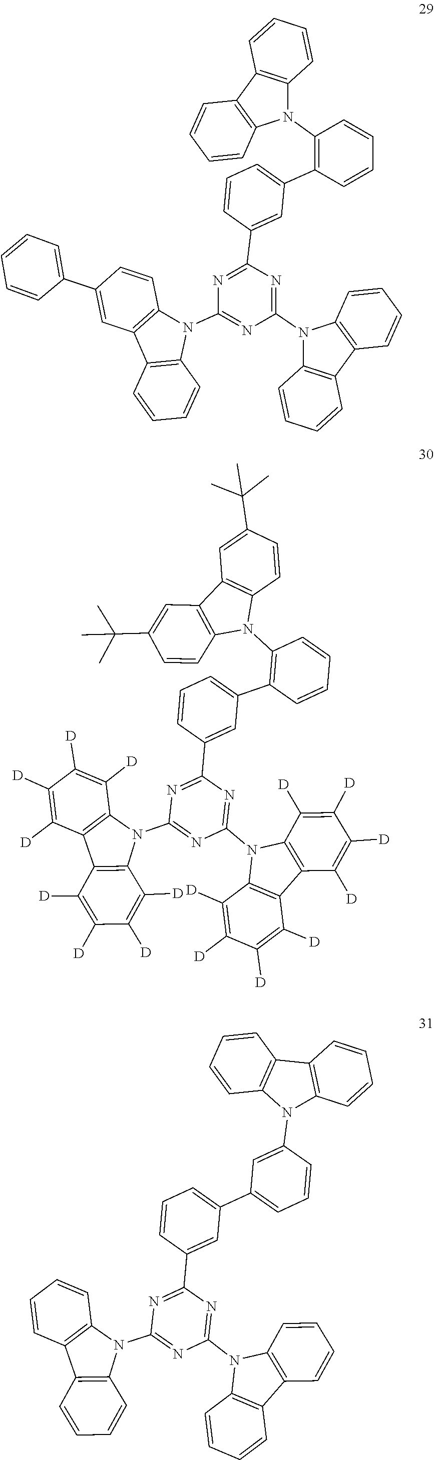 [SMILES: c1ccc(-c2ccc3c(c2)c2ccccc2n3-c2nc(-c3cccc(-c4ccccc4-n4c5ccccc5c5ccccc54)c3)nc(-n3c4ccccc4c4ccccc43)n2)cc1 ; [2H]c1c([2H])c([2H])c2c(c1[2H])c1c([2H])c([2H])c([2H])c([2H])c1n2-c1nc(-c2cccc(-c3ccccc3-n3c4ccc(C(C)(C)C)cc4c4cc(C(C)(C)C)ccc43)c2)nc(-n2c3c([2H])c([2H])c([2H])c([2H])c3c3c([2H])c([2H])c([2H])c([2H])c32)n1 ; c1cc(-c2cccc(-n3c4ccccc4c4ccccc43)c2)cc(-c2nc(-n3c4ccccc4c4ccccc43)nc(-n3c4ccccc4c4ccccc43)n2)c1]

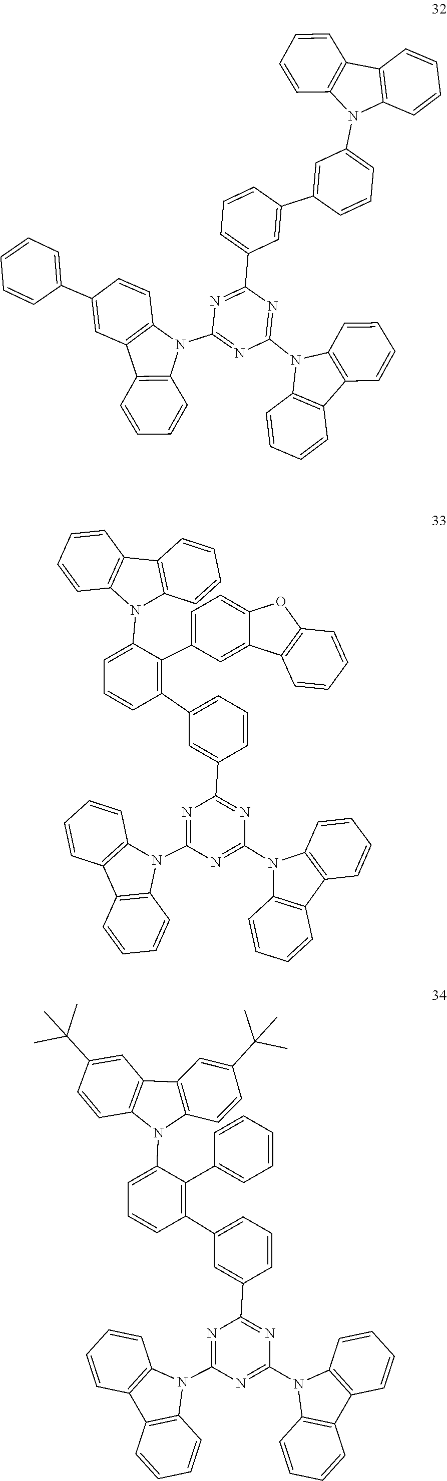 [SMILES: c1ccc(-c2ccc3c(c2)c2ccccc2n3-c2nc(-c3cccc(-c4cccc(-n5c6ccccc6c6ccccc65)c4)c3)nc(-n3c4ccccc4c4ccccc43)n2)cc1 ; c1cc(-c2nc(-n3c4ccccc4c4ccccc43)nc(-n3c4ccccc4c4ccccc43)n2)cc(-c2cccc(-n3c4ccccc4c4ccccc43)c2-c2ccc3oc4ccccc4c3c2)c1 ; CC(C)(C)c1ccc2c(c1)c1cc(C(C)(C)C)ccc1n2-c1cccc(-c2cccc(-c3nc(-n4c5ccccc5c5ccccc54)nc(-n4c5ccccc5c5ccccc54)n3)c2)c1-c1ccccc1]

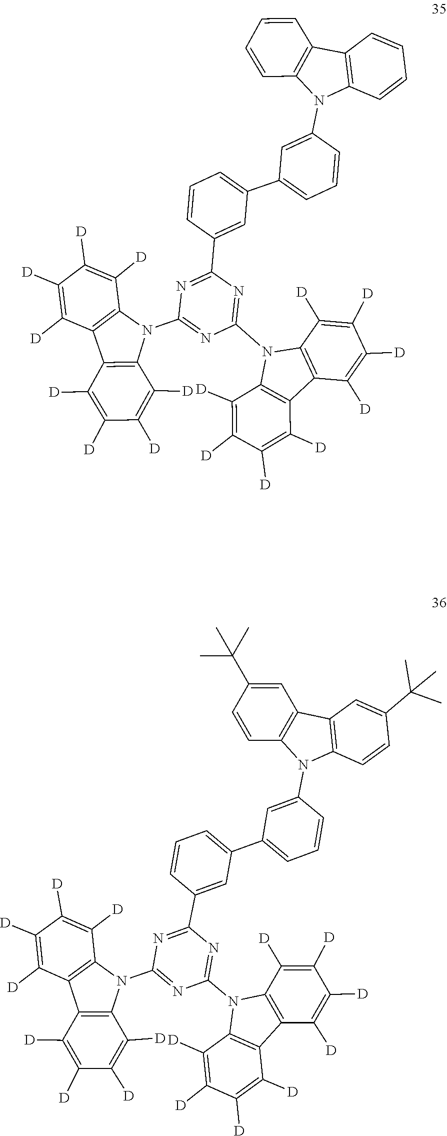 [SMILES: [2H]c1c([2H])c([2H])c2c(c1[2H])c1c([2H])c([2H])c([2H])c([2H])c1n2-c1nc(-c2cccc(-c3cccc(-n4c5ccccc5c5ccccc54)c3)c2)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2cccc(-c3cccc(-n4c5ccc(C(C)(C)C)cc5c5cc(C(C)(C)C)ccc54)c3)c2)nc(-n2c3c([2H])c([2H])c([2H])c([2H])c3c3c([2H])c([2H])c([2H])c([2H])c32)n1]

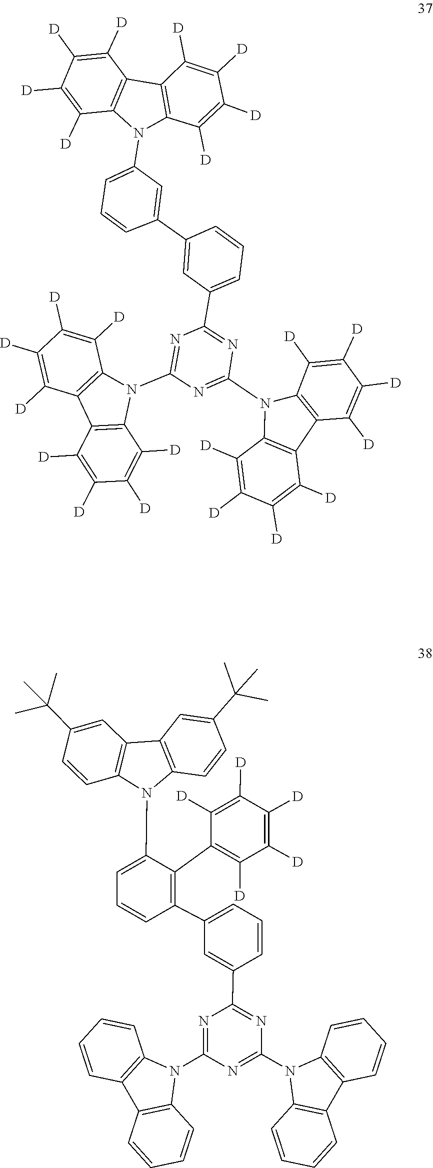 [SMILES: [2H]c1c([2H])c([2H])c2c(c1[2H])c1c([2H])c([2H])c([2H])c([2H])c1n2-c1cccc(-c2cccc(-c3nc(-n4c5c([2H])c([2H])c([2H])c([2H])c5c5c([2H])c([2H])c([2H])c([2H])c54)nc(-n4c5c([2H])c([2H])c([2H])c([2H])c5c5c([2H])c([2H])c([2H])c([2H])c54)n3)c2)c1 ; [2H]c1c([2H])c([2H])c(-c2c(-c3cccc(-c4nc(-n5c6ccccc6c6ccccc65)nc(-n5c6ccccc6c6ccccc65)n4)c3)cccc2-n2c3ccc(C(C)(C)C)cc3c3cc(C(C)(C)C)ccc32)c([2H])c1[2H]]

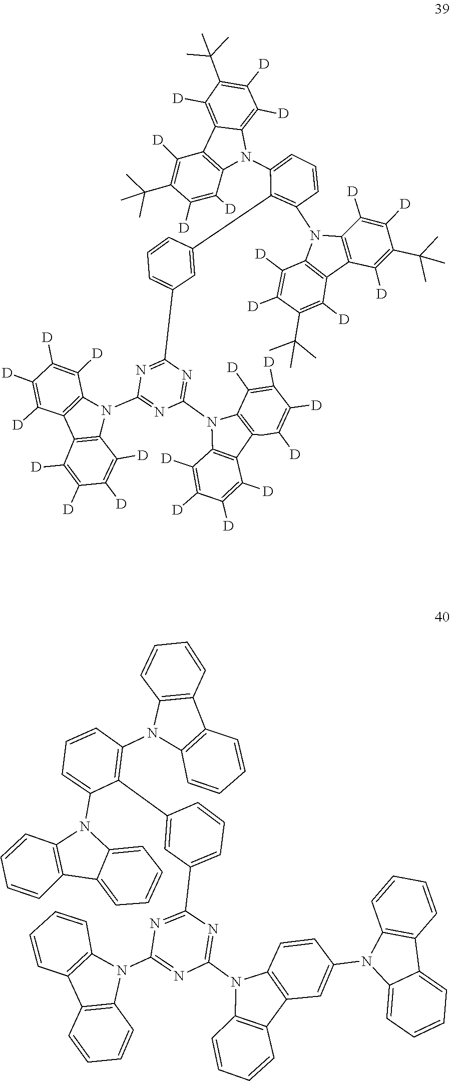 [SMILES: [2H]C1=C([2H])C([2H])=C([2H])c2c(c3c([2H])c([2H])c([2H])c([2H])c3n2-c2nc(-c3cccc(-c4c(-n5c6c([2H])c([2H])c(C(C)(C)C)c([2H])c6c6c([2H])c(C(C)(C)C)c([2H])c([2H])c65)cccc4-n4c5c([2H])c([2H])c(C(C)(C)C)c([2H])c5c5c([2H])c(C(C)(C)C)c([2H])c([2H])c54)c3)nc(-n3c4c([2H])c([2H])c([2H])c([2H])c4c4c([2H])c([2H])c([2H])c([2H])c43)n2)C1 ; c1cc(-c2nc(-n3c4ccccc4c4ccccc43)nc(-n3c4ccccc4c4cc(-n5c6ccccc6c6ccccc65)ccc43)n2)cc(-c2c(-n3c4ccccc4c4ccccc43)cccc2-n2c3ccccc3c3ccccc32)c1]

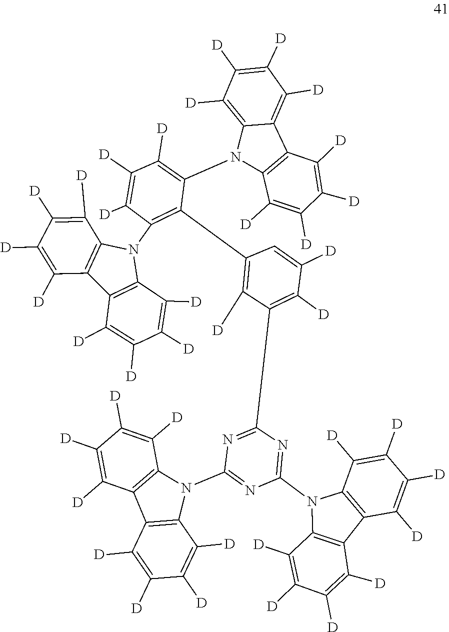 [SMILES: [2H]C1=C([2H])c2c(n(-c3c([2H])c([2H])c([2H])c(-n4c5c([2H])c([2H])c([2H])c([2H])c5c5c([2H])c([2H])c([2H])c([2H])c54)c3-c3cc([2H])c([2H])c(-c4nc(-n5c6c([2H])c([2H])c([2H])c([2H])c6c6c([2H])c([2H])c([2H])c([2H])c65)nc(-n5c6c([2H])c([2H])c([2H])c([2H])c6c6c([2H])c([2H])c([2H])c([2H])c65)n4)c3[2H])c3c([2H])c([2H])c([2H])c([2H])c23)C([2H])=C([2H])C1]

The nitrogen-containing compound of an embodiment may include a triazine group as a central structure, and three carbazole groups directly or indirectly bonded to the triazine group. Two carbazole group among the three carbazole groups may be directly bonded to the triazine group, and the remaining one carbazole group may be bonded to the triazine group via a biphenyl group.

In an embodiment, the emission layer EML may include a host and a dopant. The nitrogen-containing compound of an embodiment may be utilized as a host material in the emission layer EML. The carbazole group may be an electron donating group. The nitrogen-containing compound of an embodiment containing the carbazole group has a decrease in the band gap, and thus may prevent or reduce the deterioration of a dopant when the energy is transferred to the dopant. The nitrogen-containing compound of an embodiment containing the biphenyl group has a relatively large volume, and thus it may be prevented or reduced for the nitrogen-containing compound and the dopant to form an exciplex. In some embodiments, the nitrogen-containing compound of an embodiment may have relatively high lowest triplet excitation energy level (T1 level). Thus, when the nitrogen-containing compound is utilized as a phosphorescence emitting host material, the triplet excitons may be prevented or reduced from being annihilated. In an embodiment, the light emitting element ED including the nitrogen-containing compound may have a decrease in the driving voltage and improvement in the efficiency. However, the embodiment of the present disclosure is not limited thereto, but the nitrogen-containing compound may be utilized as a fluorescence emitting host material.

For example, the emission layer EML may include a single host and a single dopant. In some embodiments, the emission layer EML may include at least two hosts, a sensitizer, and a dopant. More specifically, the emission layer EML may include a hole transporting host and an electron transporting host. The emission layer EML of an embodiment may include the nitrogen-containing compound of an embodiment as a host. More specifically, the nitrogen-containing compound of an embodiment may be utilized as an electron transport host material.

The emission layer EML may include a phosphorescent sensitizer or a thermally activated delayed fluorescence (TADF) sensitizer as a sensitizer. For example, the phosphorescent sensitizer may be a material including a metal complex. The TADF sensitizer may include a compound represented by Formula F-c which will be described later. However, this is an example, and the phosphorescent sensitizer and TADF sensitizer are not limited thereto.

When the emission layer EML includes a hole transporting host, an electron transporting host, a sensitizer, and a dopant, the hole transporting host and the electron transporting host may form an exciplex, and the energy may be transferred from the exciplex to the sensitizer and from the sensitizer to the dopant, and thus light may be emitted. However, this is an example, and materials included in the emission layer EML are not limited thereto.

In the emission layer EML, the hole transporting host and the electron transporting host may form an exciplex. In this case, a triplet energy of the exciplex formed by the hole transporting host and the electron transporting host may correspond to the difference between a lowest unoccupied molecular orbital (LUMO) energy level of the electron transporting host and a highest occupied molecular orbital (HOMO) energy level of the hole transporting host.

For example, the absolute value of the triplet energy (T1) of the exciplex formed by the hole transporting host and the electron transporting host may be about 2.4 eV to about 3.0 eV. In some embodiments, the triplet energy of the exciplex may be a value smaller than an energy gap of each host material. The exciplex may have a triplet energy of about 3.0 eV or less that is an energy gap between the hole transporting host and the electron transporting host. However, this is an example, and the embodiment of the present disclosure is not limited thereto.

The emission layer EML of an embodiment may further include the second compound represented by Formula HT-1. The second compound may be utilized as a hole transporting host material.

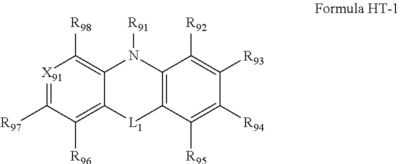

In Formula HT-1, L₁ may be a direct linkage, CR₉₉R₁₀₀, or SiR₁₀₁R₁₀₂. X₉₁ may be N or CR₁₀₃. For example, when L₁ is a direct linkage and X₉₁ is CR₁₀₃, the second compound represented by Formula HT-1 may have a carbazole core.

R₉₁ to R₁₀₃ may each independently be a hydrogen atom, a deuterium atom, a cyano group, a substituted or unsubstituted silyl group, a substituted or unsubstituted oxy group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, and/or may be bonded to an adjacent group to form a ring, For example, R₉₁ may be a substituted phenyl group, an unsubstituted dibenzofuran group, or a substituted fluorenyl group. Any one among R₉₂ to R₉₈ may be a substituted or unsubstituted carbazole group. R₉₄ and R₉₅ may be bonded to each other to form a ring.

The second compound may be represented by any one among the compounds in Compound Group 2. In Compound Group 2, D is a deuterium atom, and Ph is a phenyl group.

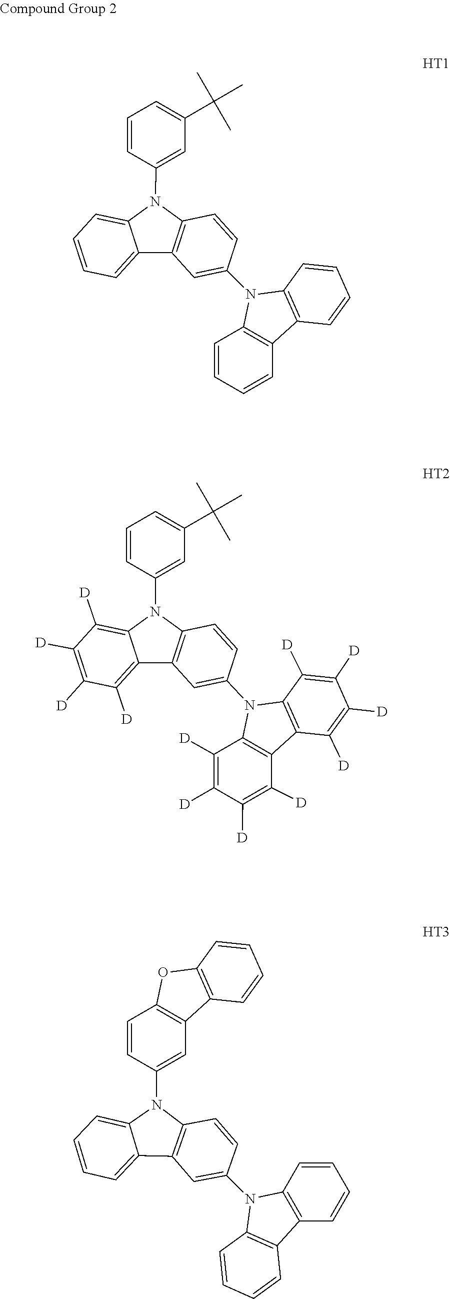 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

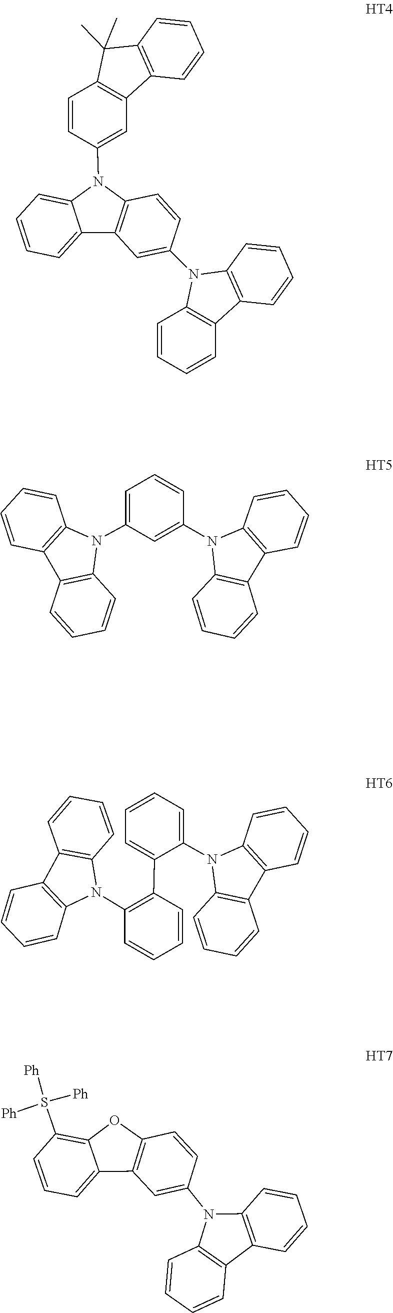 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc(S(c2ccccc2)(c2ccccc2)c2cccc3c2oc2ccc(-n4c5ccccc5c5ccccc54)cc23)cc1]

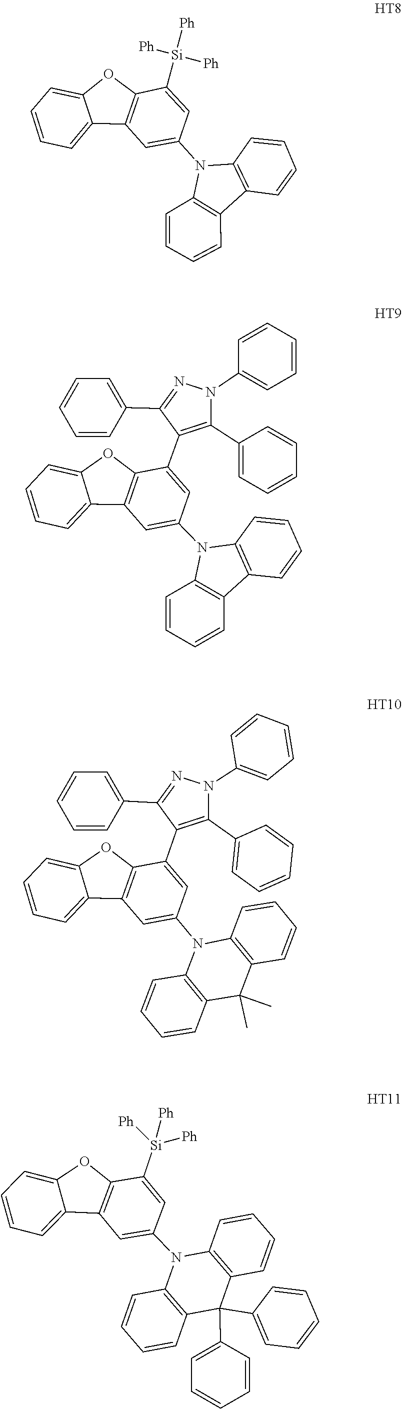 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

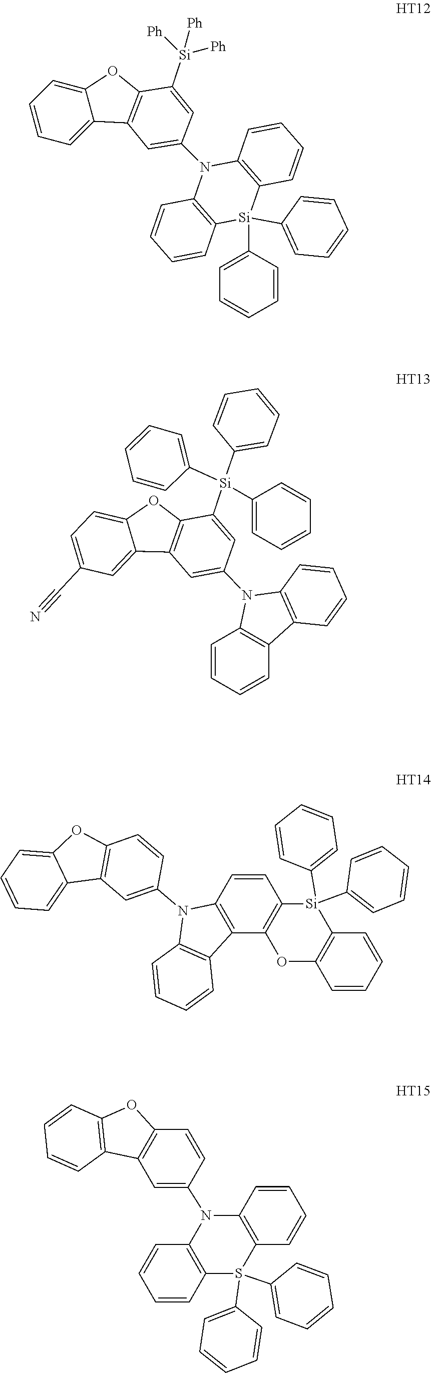 [SMILES: c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc(S2(c3ccccc3)c3ccccc3N(c3ccc4oc5ccccc5c4c3)c3ccccc32)cc1]

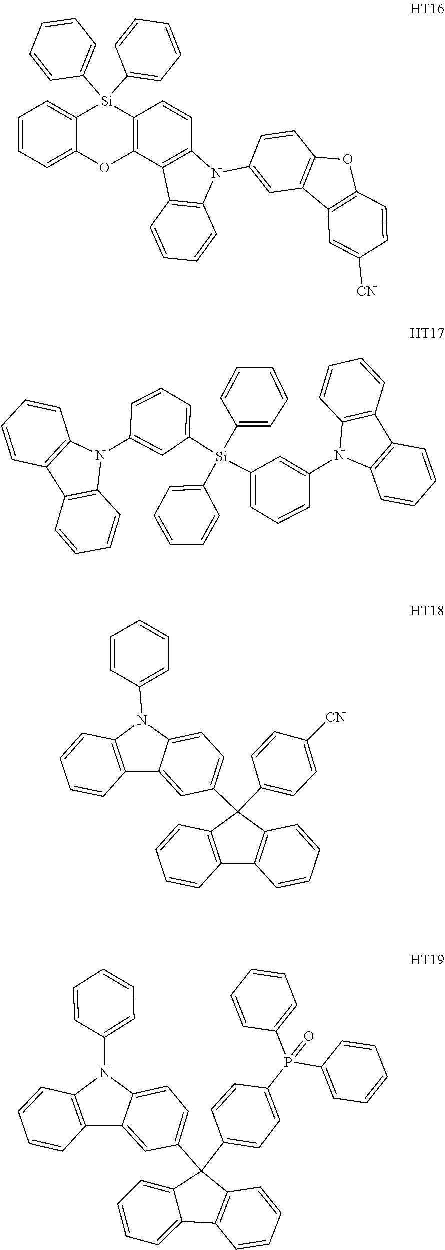 [SMILES: N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1]

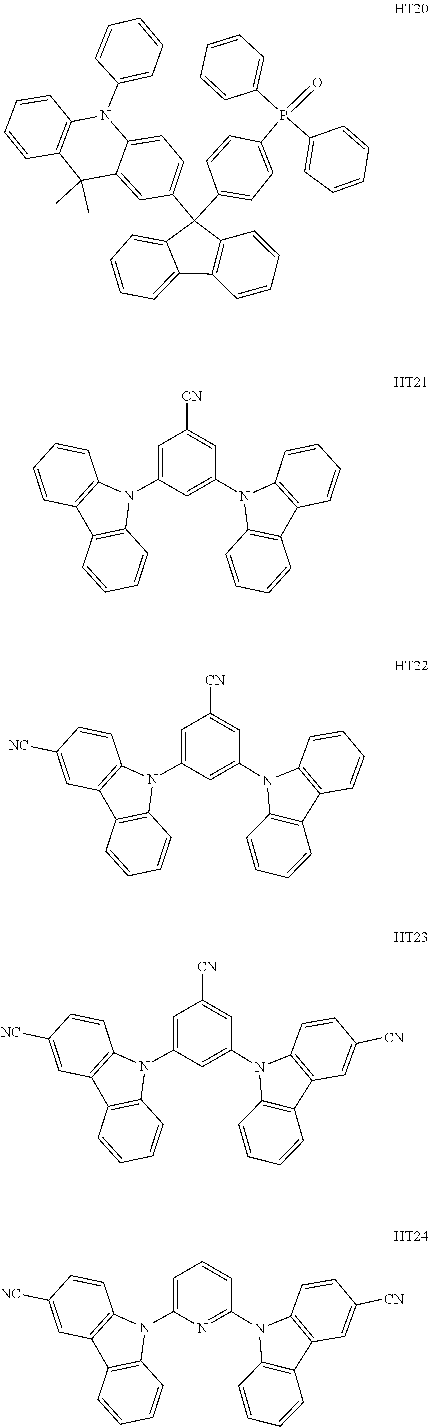 [SMILES: CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1]

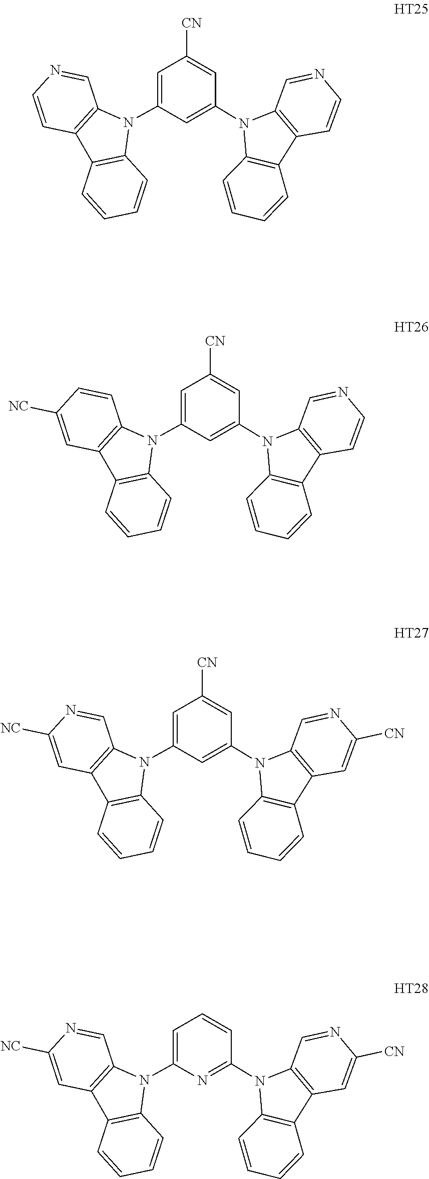 [SMILES: N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1]

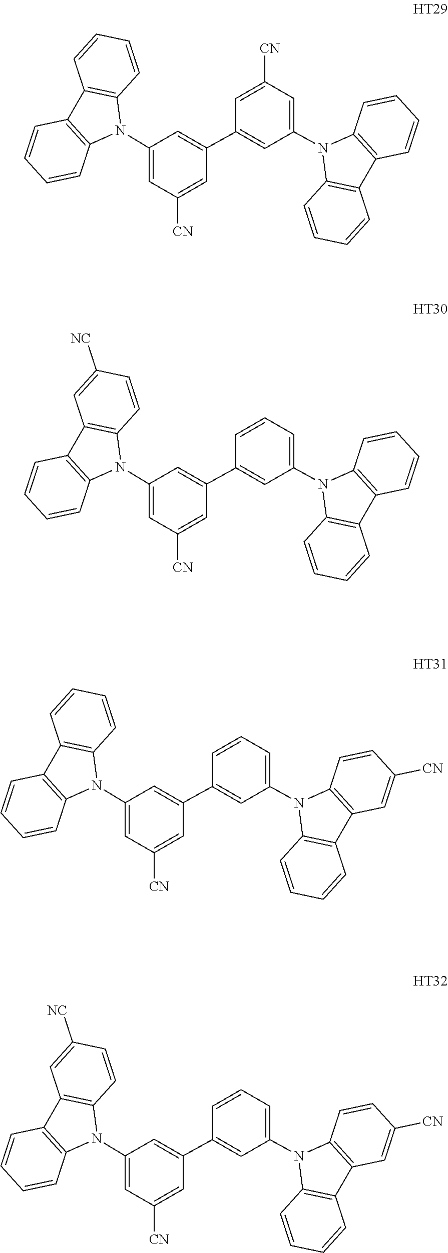 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1]

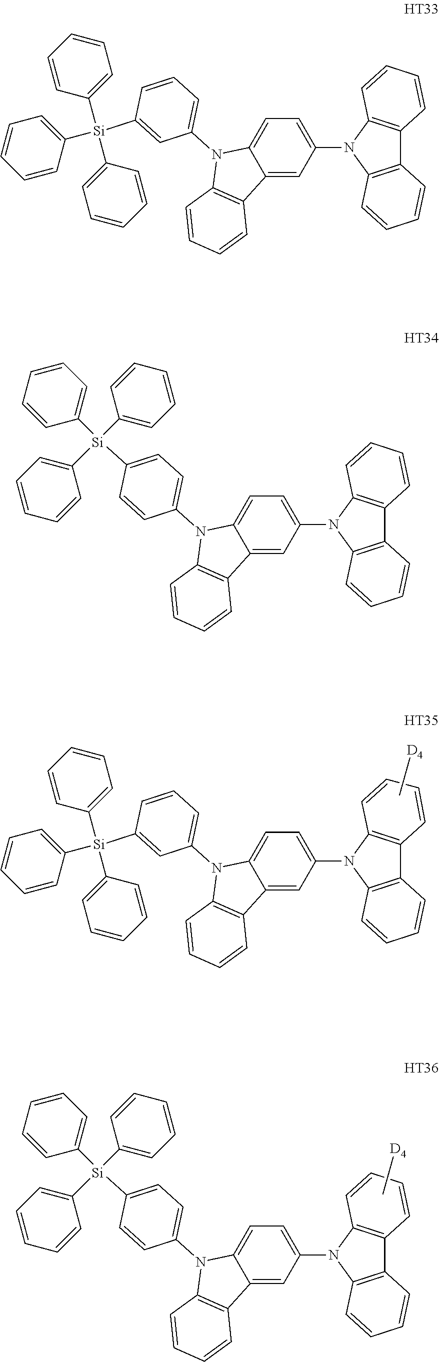

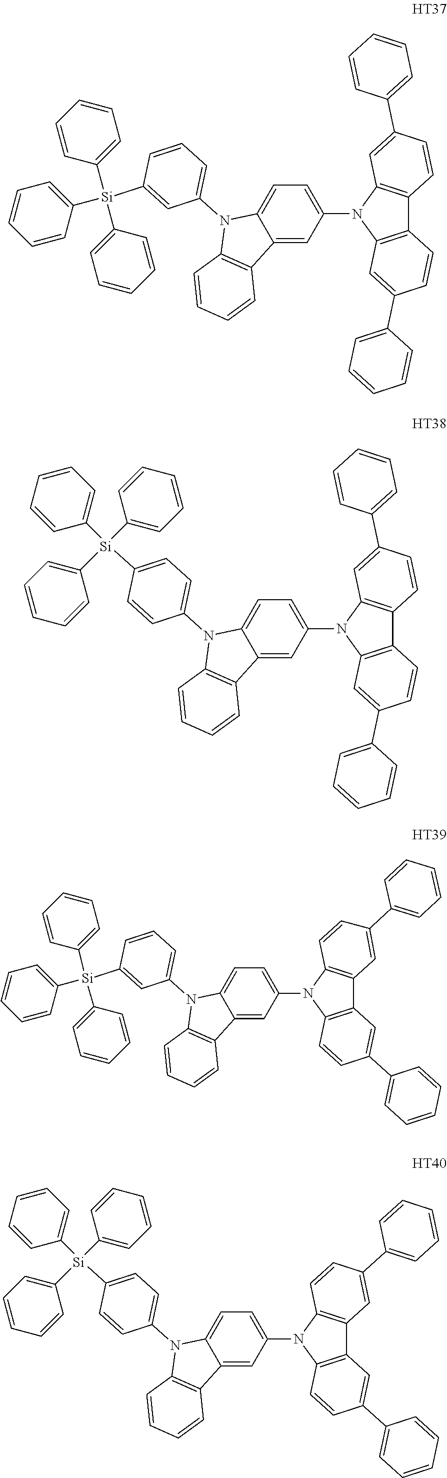 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1 ; c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1]

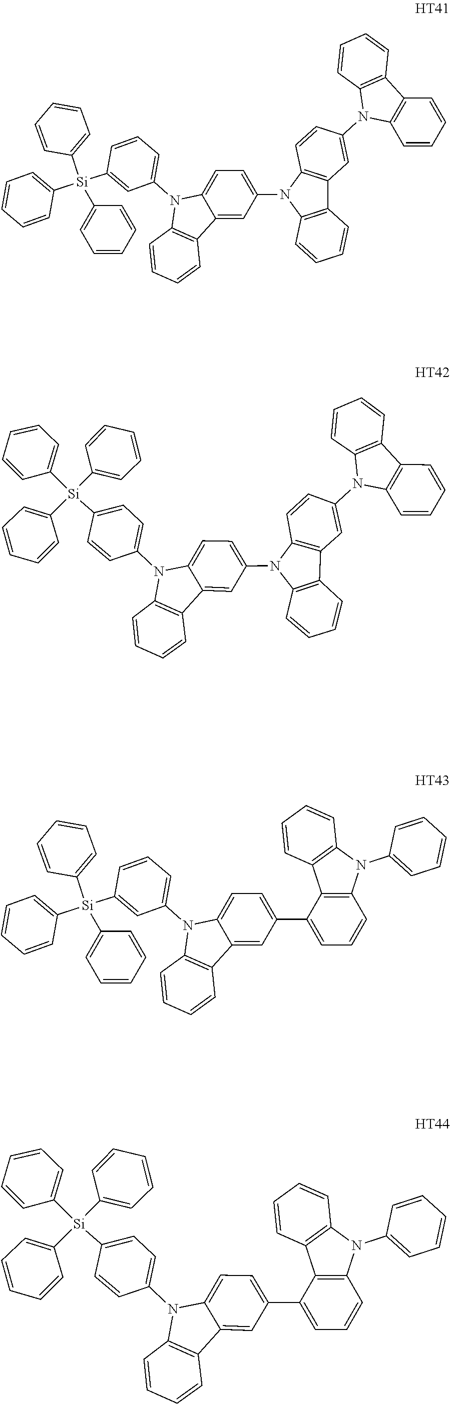 [SMILES: c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; C1=Cc2c(n(-c3ccc4c(c3)c3ccccc3n4-c3ccc([Si](c4ccccc4)(c4ccccc4)c4ccccc4)cc3)c3ccc(-n4c5ccccc5c5ccccc54)cc23)C=CC1 ; c1ccc(-n2c3ccccc3c3c(-c4ccc5c(c4)c4ccccc4n5-c4cccc([Si](c5ccccc5)(c5ccccc5)c5ccccc5)c4)cccc32)cc1 ; c1ccc(-n2c3ccccc3c3c(-c4ccc5c(c4)c4ccccc4n5-c4ccc([Si](c5ccccc5)(c5ccccc5)c5ccccc5)cc4)cccc32)cc1]

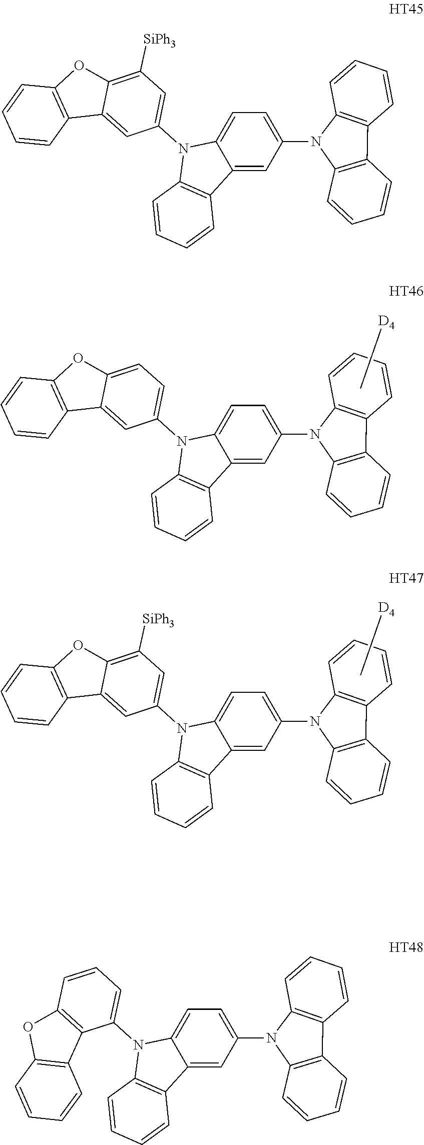

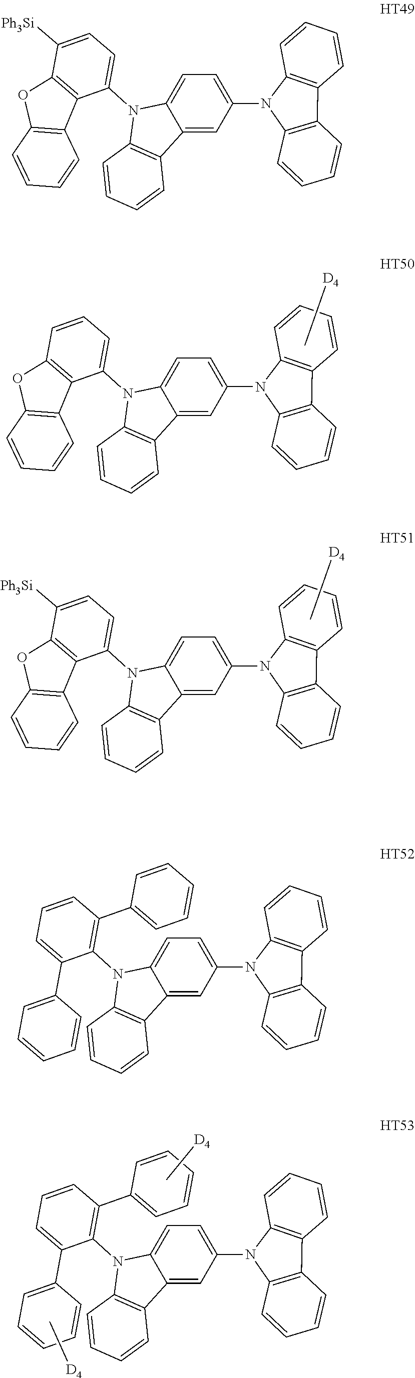

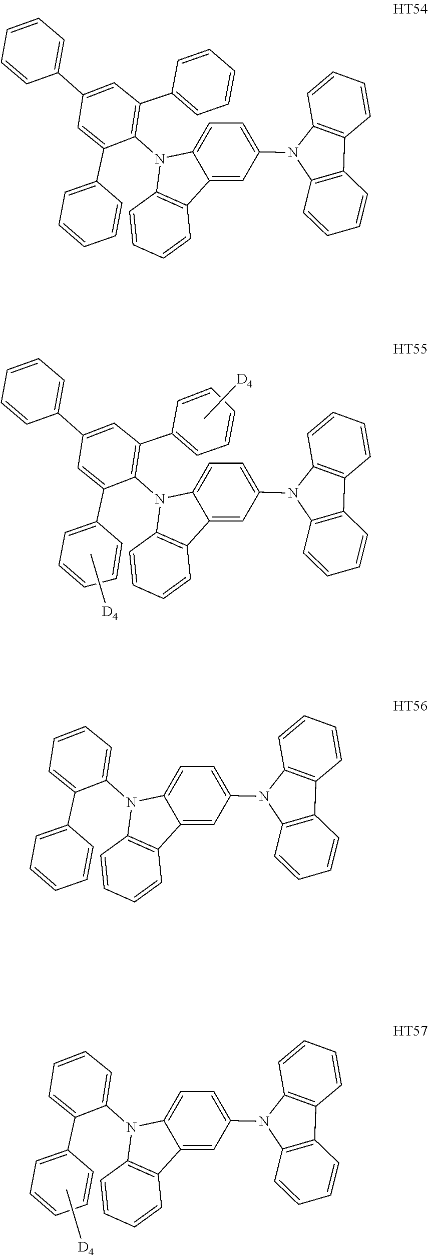

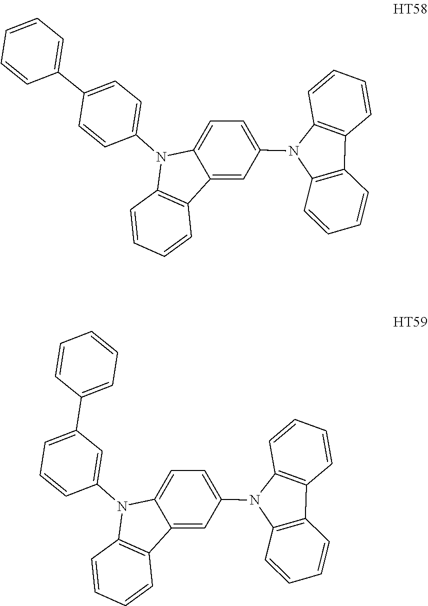 [SMILES: c1ccc(-c2ccc(-n3c4ccccc4c4cc(-n5c6ccccc6c6ccccc65)ccc43)cc2)cc1 ; c1ccc(-c2cccc(-n3c4ccccc4c4cc(-n5c6ccccc6c6ccccc65)ccc43)c2)cc1]

The emission layer EML of an embodiment may further include a third compound represented by Formula M-a. The third compound may be utilized as a phosphorescent dopant material.

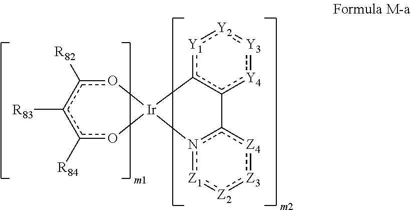

In Formula M-a, Y₁ to Y₄ and Z₁ to Z₄ may each independently be CR₈₁ or N. R₈₁ to R₈₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring.

In Formula M-a, m1 may be 0 or 1, and m2 may be 2 or 3. When m1 is 0, m2 may be 3, and when m1 is 1, m2 may be 2.

The compound represented by Formula M-a may be represented by any one among Compound M-a1 to Compound M-a25. However, Compounds M-a1 to M-a25 are examples, and the compound represented by Formula M-a is not limited to those represented by Compounds M-a1 to M-a25.

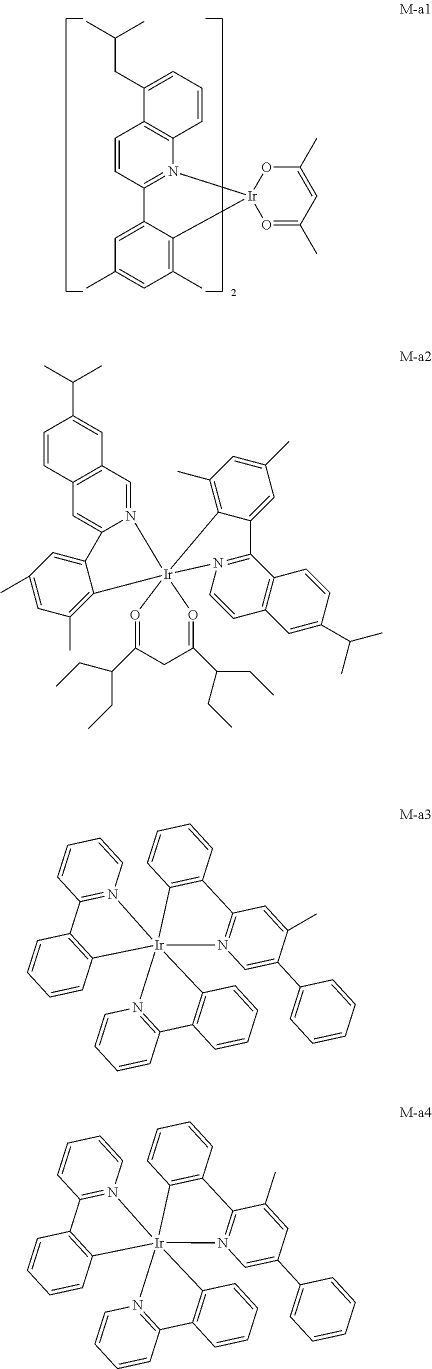

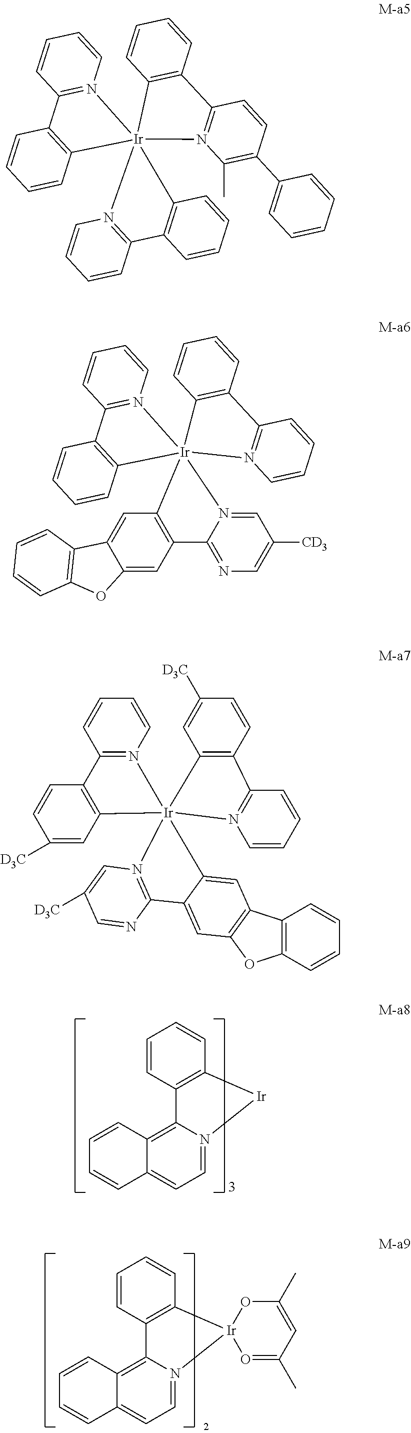

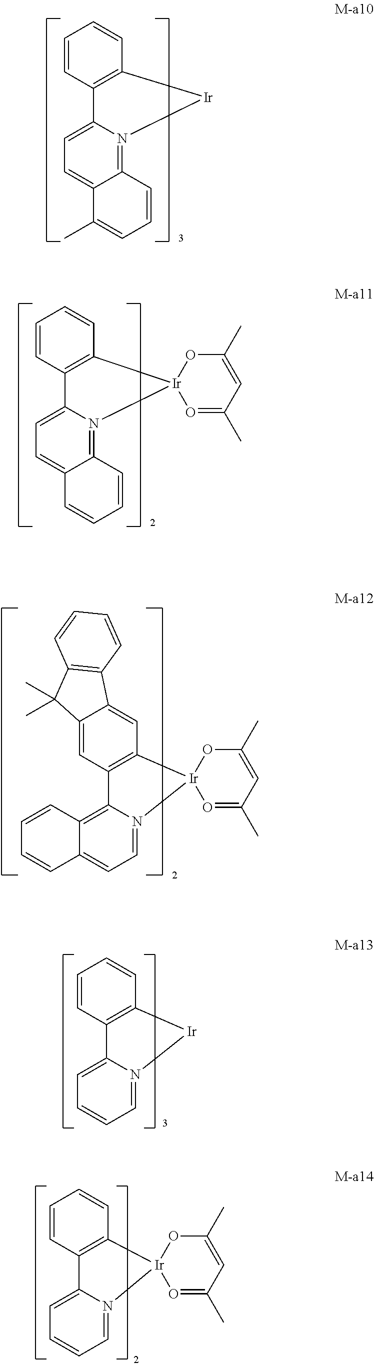

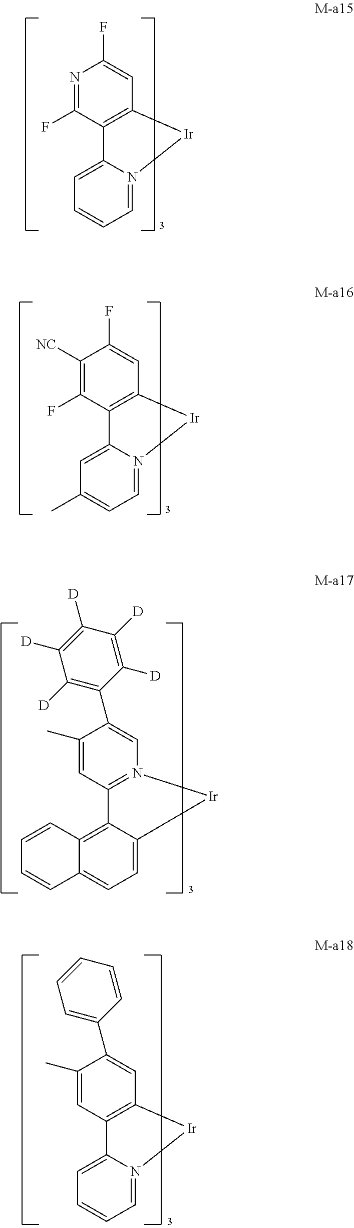

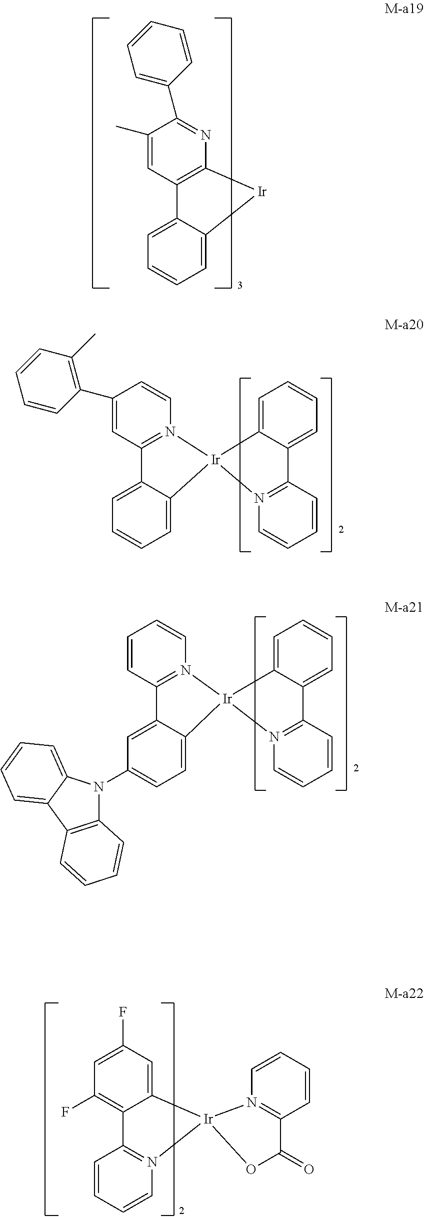

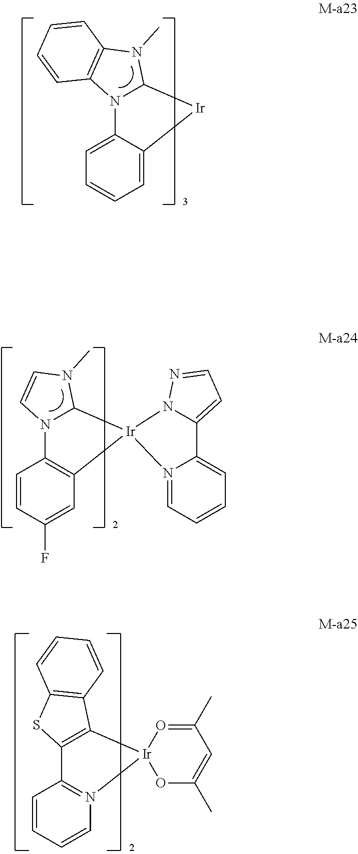

Compound M-a1 and Compound M-a2 may be utilized as a red dopant material, and Compound M-a3 to Compound M-a7 may be utilized as a green dopant material.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

The emission layer EML may further include compounds, which will be described, besides the nitrogen-containing compound of an embodiment, the second compound, and the third compound.

In the light emitting element ED of an embodiment, the emission layer EML may include an anthracene derivative, a pyrene derivative, a fluoranthene derivative, a chrysene derivative, a dehydrobenzanthracene derivative, or a triphenylene derivative. For example, the emission layer EML may include the anthracene derivative or the pyrene derivative.

The emission layer EML may include a compound represented by Formula E-1. The compound represented by Formula E-1 may be utilized as a fluorescent host material.

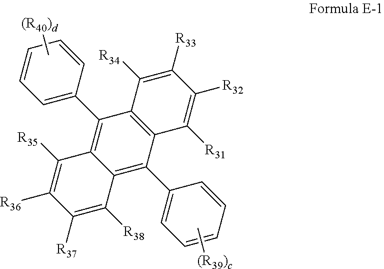

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In some embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer of 0 to 5. When c is an integer of 2 or greater, a plurality of R₃₉'s may be the same as each other or at least one may be different from the others. When d is an integer of 2 or greater, a plurality of R₄₀'s may be the same as each other or at least one may be different from the others. Formula E-1 may be represented by any one among Compound E1 to Compound E19:

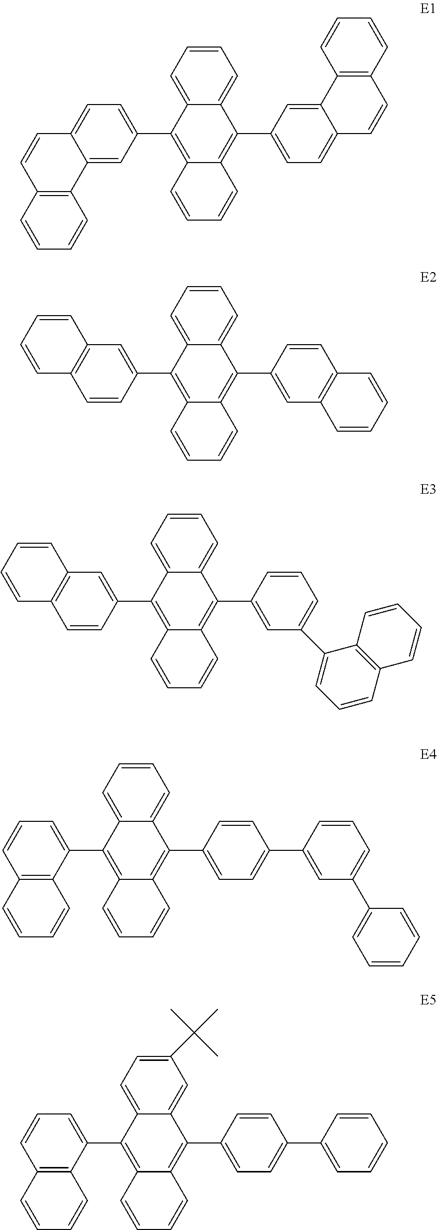

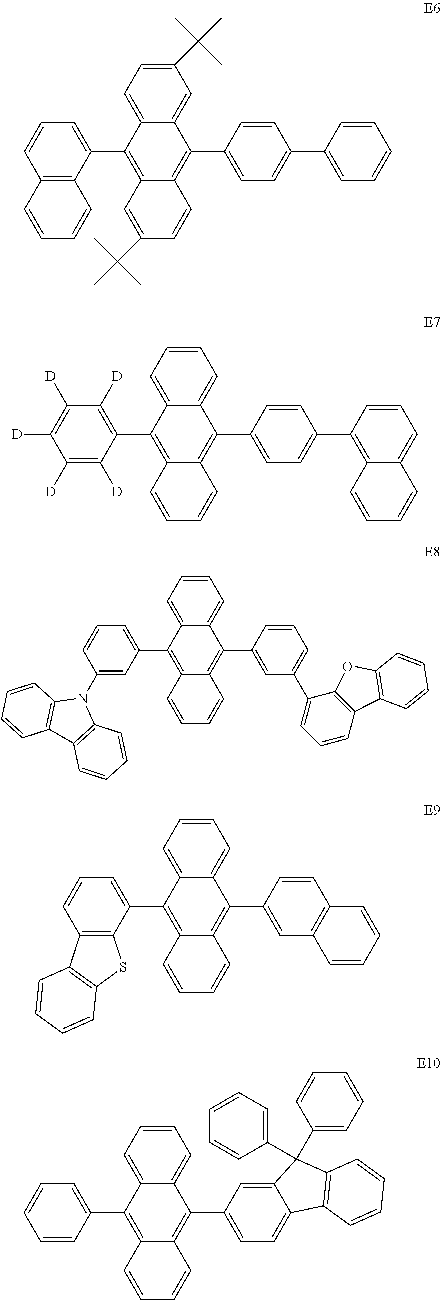

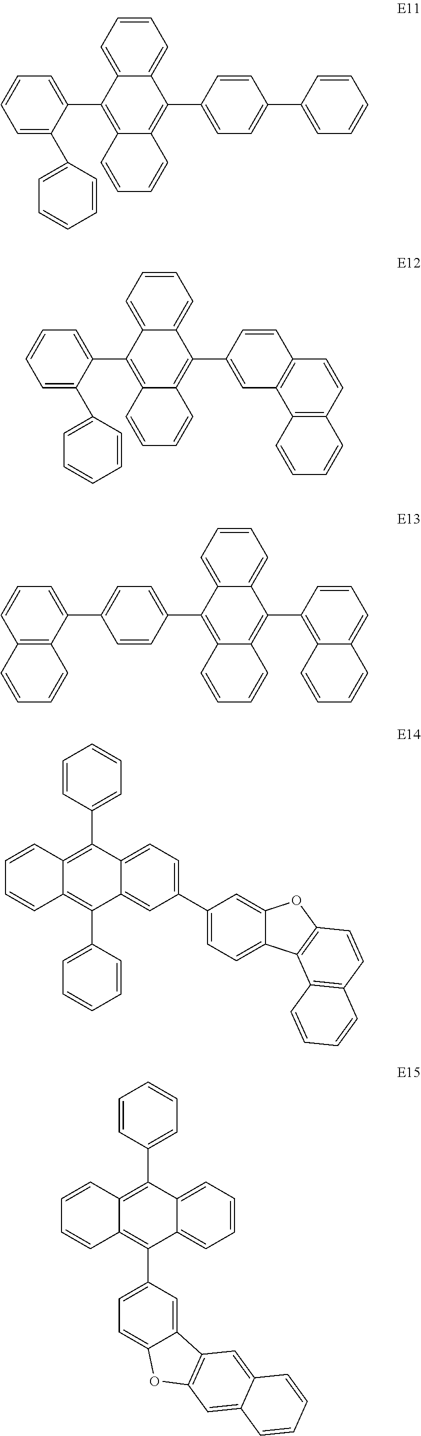

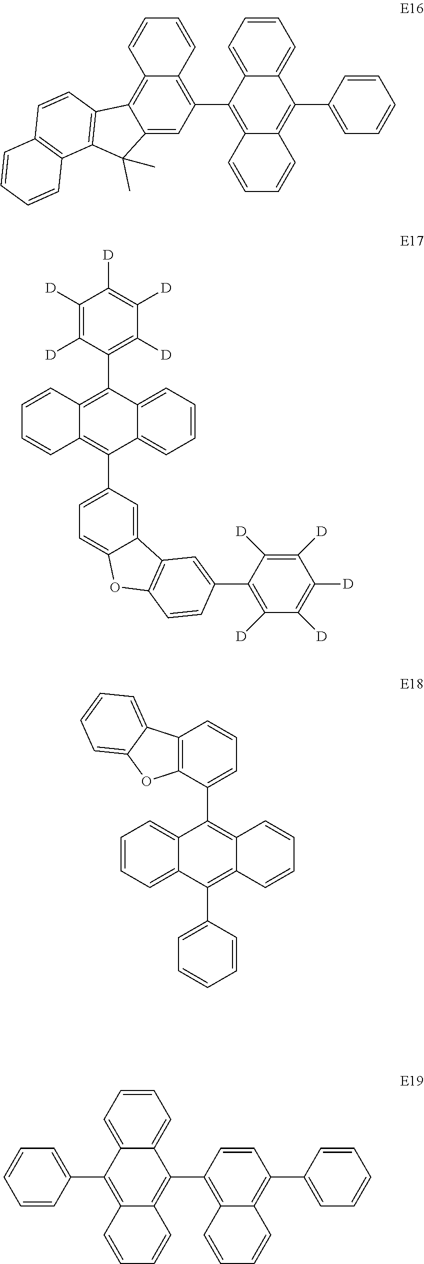

In an embodiment, the emission layer EML may include a compound represented by Formula E-2a or Formula E-2b. The compound represented by Formula E-2a or Formula E-2b may be utilized as a phosphorescent host material.

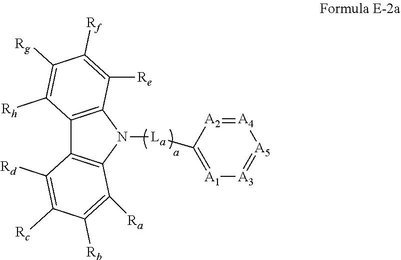

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or greater, a plurality of Lₐ's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. Rₐ to Rᵢ may be bonded to an adjacent group to form a hydrocarbon ring or a heterocycle containing N, O, S, etc., as a ring-forming atom.

In some embodiments, in Formula E-2a, two or three selected from among A₁ to A₅ may be N, and the rest may be CRᵢ.

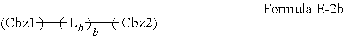

In Formula E-2b, Cbz1 and Cbz2 may each independently be an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. In some embodiments, b is an integer of 0 to 10, and when b is an integer of 2 or more, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one among the compounds of Compound Group E-2. However, the compounds listed in Compound Group E-2 are examples, and the compound represented by Formula E-2a or Formula E-2b is not limited to those represented in Compound Group E-2.

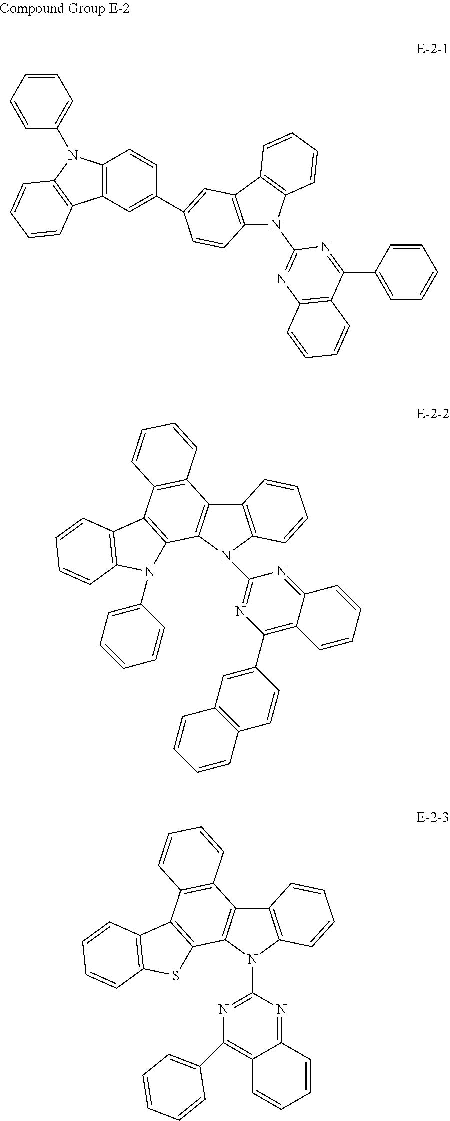 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

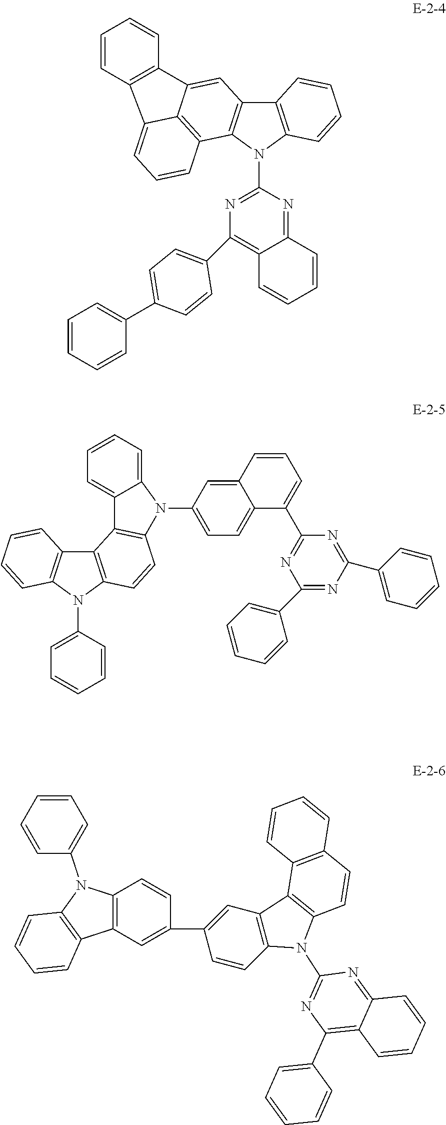 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

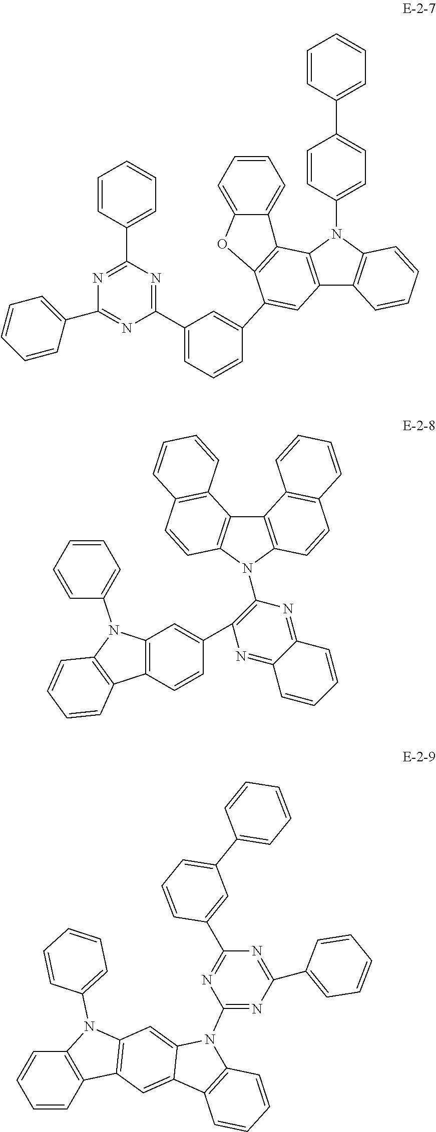 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

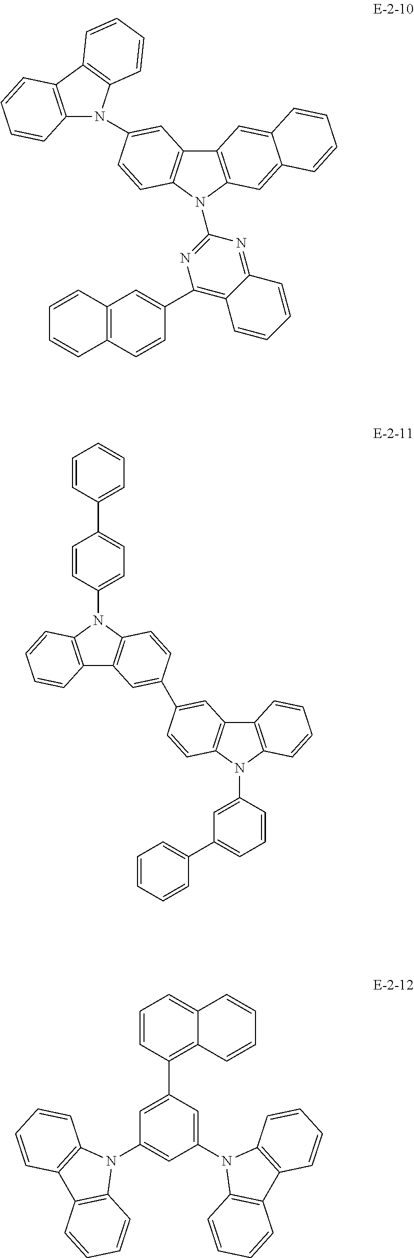 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

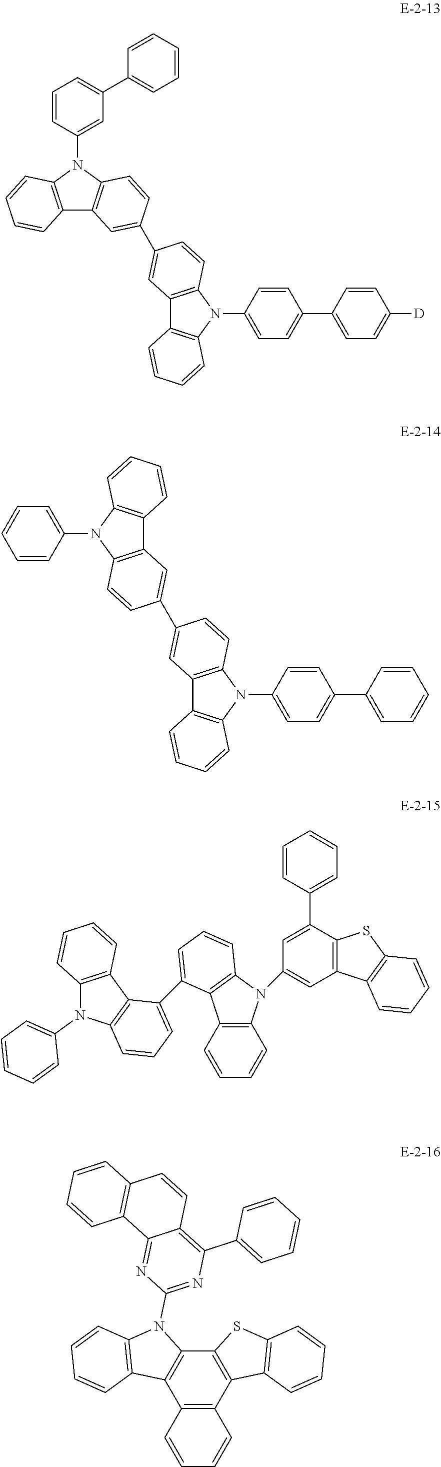 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1]

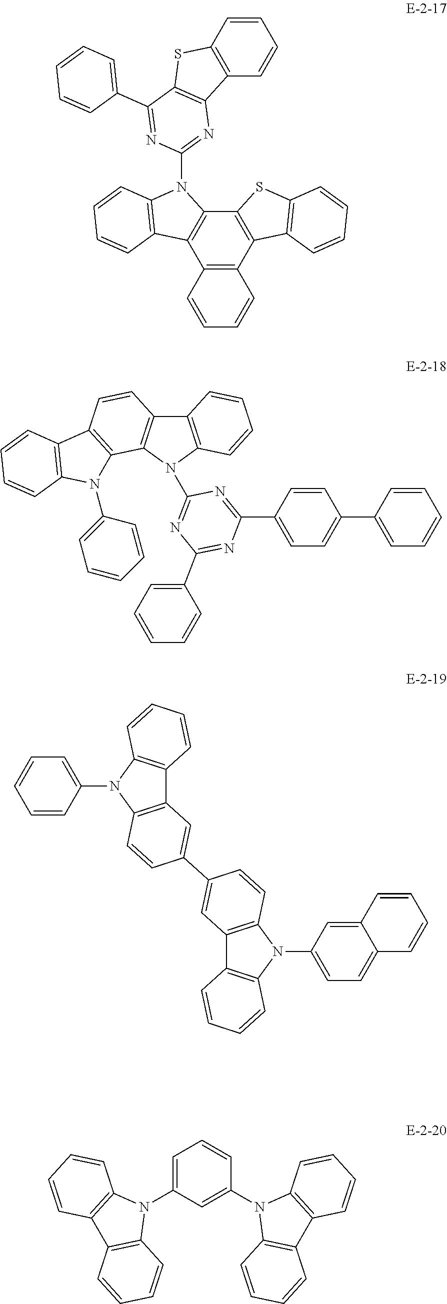 [SMILES: c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1]

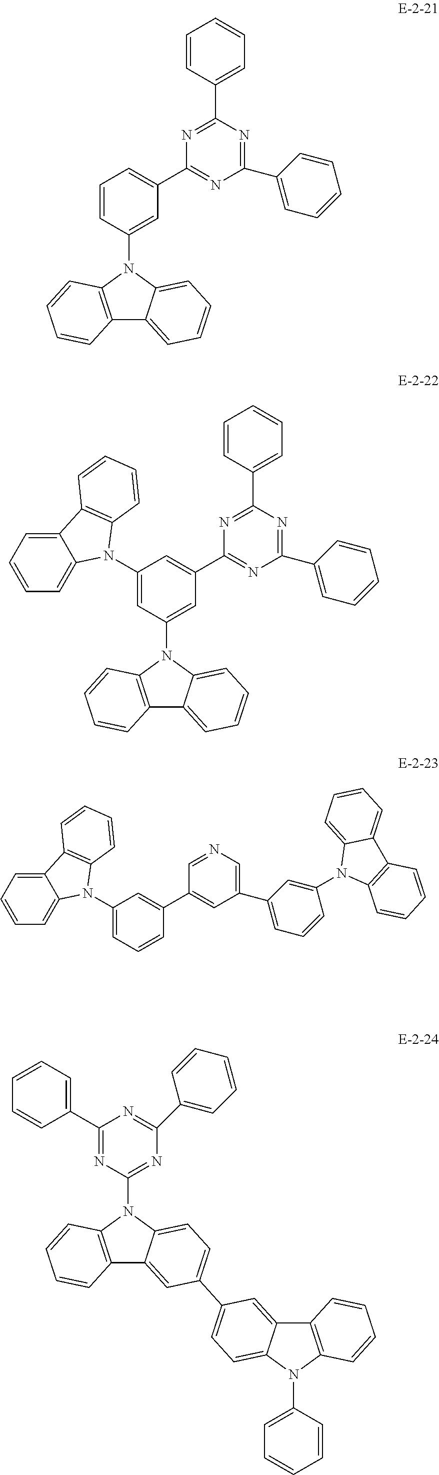 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiment of the present disclosure is not limited thereto, for example, tris(8-hydroxyquinolino)aluminum (Alq3), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), etc. may be utilized as a host material.

The emission layer EML may include the compound represented by Formula M-b. The compound represented by Formula M-b may be utilized as a phosphorescent dopant material.

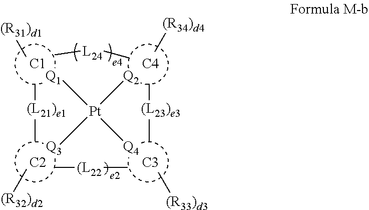

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ may each independently be a direct linkage, *—O—*, *—S—*,

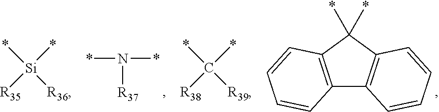

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1.

In Formula M-b, R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In some embodiments, d1 to d4 may each independently be an integer of 0 to 4.

The compound represented by Formula M-b may be utilized as a blue phosphorescent dopant or a green phosphorescent dopant. The compound represented by Formula M-b may be represented by any one among the compounds. However, the compounds are examples, and the compound represented by Formula M-b is not limited to those represented by the compounds.

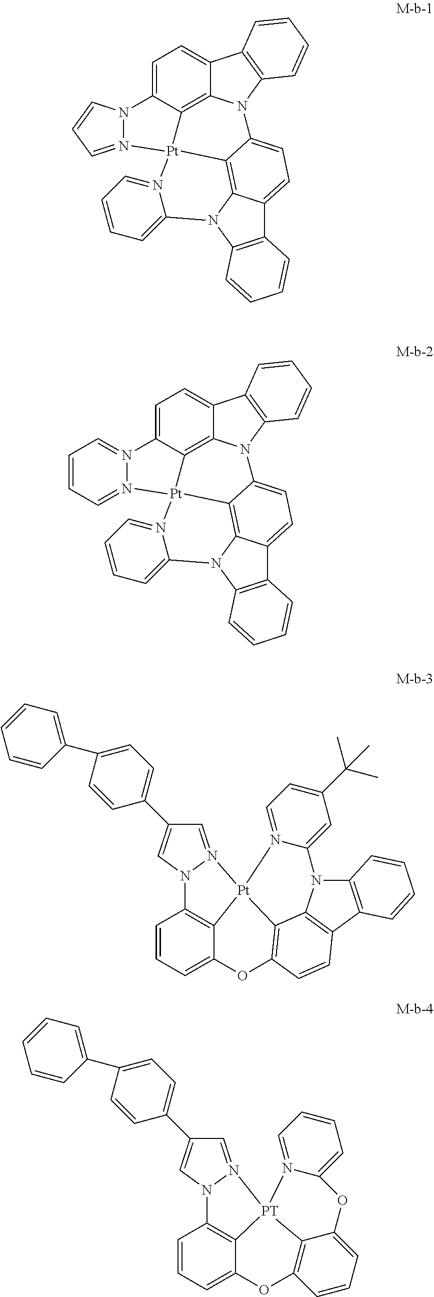

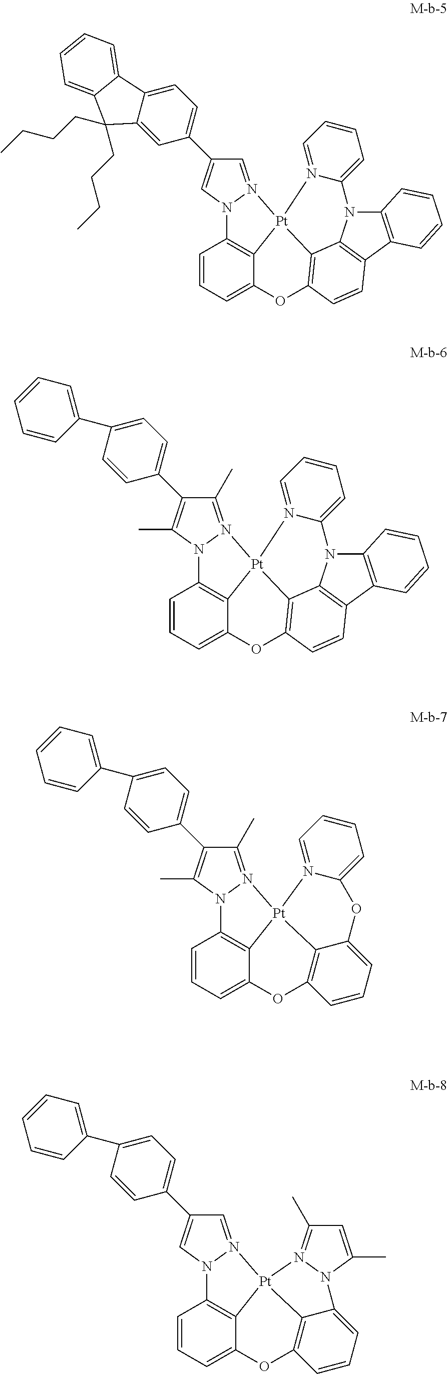

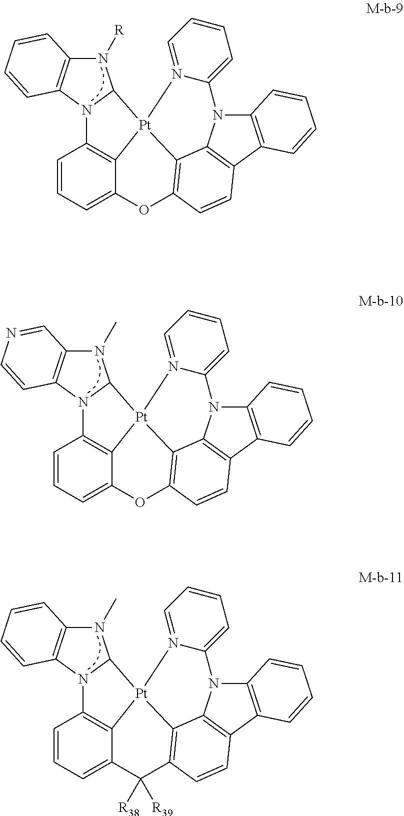

In the compounds, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one among Formula F-a to Formula F-c. The compound represented by Formula F-a or Formula F-c may be utilized as a fluorescence dopant material.

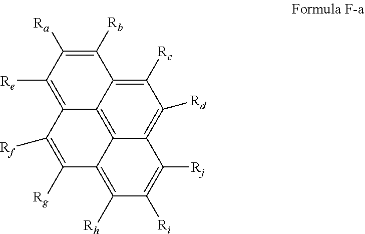

In Formula F-a above, two selected from among Rₐ to Rⱼ may each independently be substituted with *—NAr₁Ar₂. The others, which are not substituted with *—NAr₁Ar₂ among Rₐ to Rⱼ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

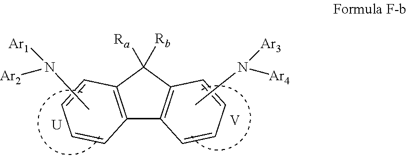

In Formula F-b above, Rₐ and Rb may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. In Formula F-b, the number of rings represented by U and V may each independently be 0 or 1.

For example, in Formula F-b, it refers to that when the number of U or V is 1, one ring constitutes a fused ring at a portion indicated by U or V, and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring having a fluorene core in Formula F-b may be a cyclic compound having three rings. In some embodiments, when each number of U and V is 1, the fused ring having a fluorene core in Formula F-b may be a cyclic compound having five rings.

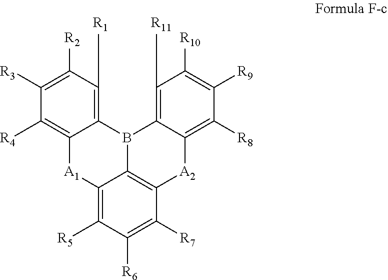

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boron group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring.

In Formula F-c, A₁ and A₂ may each independently be bonded to substituents of an adjacent ring to form a fused ring. For example, when A₁ and A₂ may each independently be NRₘ, A₁ may be bonded to R₄ or R₅ to form a ring. In some embodiments, A₂ may be bonded to R₇ or R₈ to form a ring.

In an embodiment, the emission layer EML may include, as a suitable dopant material, a styryl derivative (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and a derivative thereof (e.g., 2,5,8,11-tetra-t-butylperylene (TBP)), pyrene and a derivative thereof (e.g., 1,1′-dipyrene, 1,4-dipyrenylbenzene, 1,4-bis(N,N-diphenylamino)pyrene), etc.

The emission layer EML may include a suitable phosphorescent dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be utilized as a phosphorescent dopant. For example, iridium(III) bis(4,6-difluorophenylpyridinato-N,C2′)picolinate (Firpic), bis(2,4-difluorophenylpyridinato)-tetrakis(1-pyrazolyl)borate iridium(III) (FIr6), or platinum octaethyl porphyrin (PtOEP) may be utilized as a phosphorescent dopant. However, the embodiment of the present disclosure is not limited thereto.

The emission layer EML may include a quantum dot material. The core of the quantum dot may be selected from a Group II-VI compound, a Group III-VI compound, a Group I-III-IV compound, a Group III-V compound, a Group III-II-V compound, a Group IV-VI compound, a Group IV element, a Group IV compound, or a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof.

The Group III-VI compound may include a binary compound such as In₂S₃ or In₂Se₃, a ternary compound such as InGaS₃ or InGaSe₃, or a combination thereof.

The Group I-III-VI compound may be selected from a ternary compound selected from the group consisting of AgInS, AgInS₂, CuInS, CuInS₂, AgGaS₂, CuGaS₂ CuGaO₂, AgGaO₂, AgAIO₂, and a mixture thereof, or a quaternary compound such as AgInGaS₂ or CuInGaS₂.

The Group III-V compound may be selected from the group consisting of a binary compound selected from the group consisting of GaN, GaP, GaAs, GaSb, AlN, AIP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. In some embodiments, the Group III-V compound may further include a Group II metal. For example, InZnP, etc., may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

In this case, the binary compound, the ternary compound, or the quaternary compound may be present in a particle with a substantially uniform concentration distribution, or may be present in substantially the same particle with a partially different concentration distribution. In some embodiments, the quantum dot may have a core/shell structure in which one quantum dot surrounds another quantum dot. The core/shell structure may have a concentration gradient in which the concentration of elements present in the shell decreases toward the core.

In some embodiments, the quantum dot may have the above-described core/shell structure including a core containing nanocrystals and a shell around (e.g., surrounding) the core. The shell of the quantum dot may serve as a protection layer to prevent or reduce the chemical deformation of the core to maintain semiconductor properties, and/or a charging layer to impart electrophoresis properties to the quantum dot. The shell may be a single layer or a multilayer. An example of the shell of the quantum dots may include a metal or non-metal oxide, a semiconductor compound, or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but the embodiment of the present disclosure is not limited thereto.

Also, examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

The quantum dot may have a full width of half maximum (FWHM) of a light emitting wavelength spectrum of about 45 nm or less, about 40 nm or less, and about 30 nm or less, and color purity or color reproducibility may be improved in the above range. In some embodiments, light emitted through such a quantum dot is emitted in all directions, and thus a wide viewing angle may be improved.

In some embodiments, although the form of a quantum dot is not particularly limited as long as it is a form commonly utilized in the art, more specifically, a quantum dot in the form of spherical, pyramidal, multi-arm, or cubic nanoparticles, nanotubes, nanowires, nanofibers, nanoparticles, etc. may be utilized.

A quantum dot may control the color of emitted light according to the particle size thereof and thus the quantum dot may have one or more suitable light emission colors such as green, red, etc.

Referring to FIGS. 3A to 3B again, the first electrode EL1 has conductivity (e.g., is a conductor). The first electrode EL1 may be formed of a metal material, a metal alloy, or a conductive compound. The first electrode EL1 may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and/or Zn, a compound of two or more selected from among these, a mixture of two or more selected from among these, or an oxide thereof.

When the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but the embodiment of the present disclosure is not limited thereto. In some embodiments, the first electrode EL1 may include the above-described metal materials, combinations of at least two metal materials of the above-described metal materials, oxides of the above-described metal materials, and/or the like, and the embodiment of the present disclosure is not limited thereto. The thickness of the first electrode EL1 may be from about 700 Å to about 10,000 Å. For example, the thickness of the first electrode EL1 may be from about 1,000 Å to about 3,000 Å.

The hole transport region HTR may be provided on the first electrode EL1. The hole transport region HTR may include at least one of a hole injection layer HIL, a hole transport layer HTL, a buffer layer or an emission-auxiliary layer, or an electron blocking layer EBL.

The hole transport region HTR may have a single layer formed of a single material, a single layer formed of a plurality of different materials, or a multilayer structure including a plurality of layers formed of a plurality of different materials. For example, the hole transport region HTR may have a single layer structure of the hole injection layer HIL or the hole transport layer HTL, or may have a single layer structure formed of a hole injection material and a hole transport material.

In some embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1. However, this is an example, and the embodiment of the present disclosure is not limited thereto.

The hole transport region HTR may be formed utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

The hole transport region HTR may include a compound represented by Formula H-1:

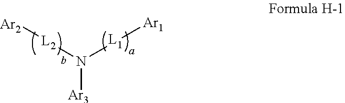

In Formula H-1 above, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, a and b may each independently be an integer of 0 to 10. In some embodiments, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms.

The compound represented by Formula H-1 above may be a monoamine compound. In some embodiments, the compound represented by Formula H-1 above may be a diamine compound in which at least one among Ar₁ to Ar₃ includes the amine group as a substituent. In some embodiments, the compound represented by Formula H-1 above may be a carbazole-based compound including a substituted or unsubstituted carbazole group in at least one of Ar or Ar₂, or a fluorene-based compound including a substituted or unsubstituted fluorene group in at least one of Ar or Ar₂.

The compound represented by Formula H-1 may be represented by any one among the compounds in Compound Group H. However, the compounds listed in Compound Group H are examples, and the compounds represented by Formula H-1 are not limited to those represented by Compound Group H:

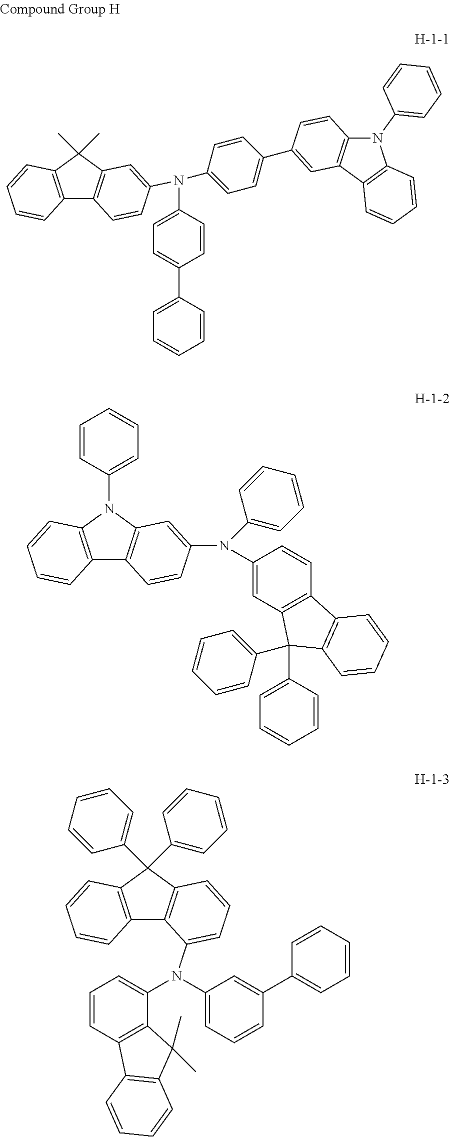 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

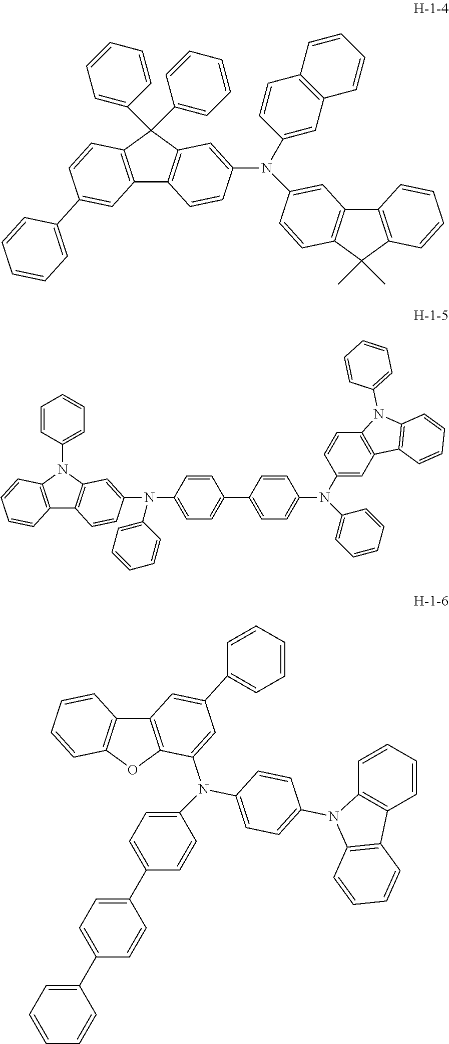 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

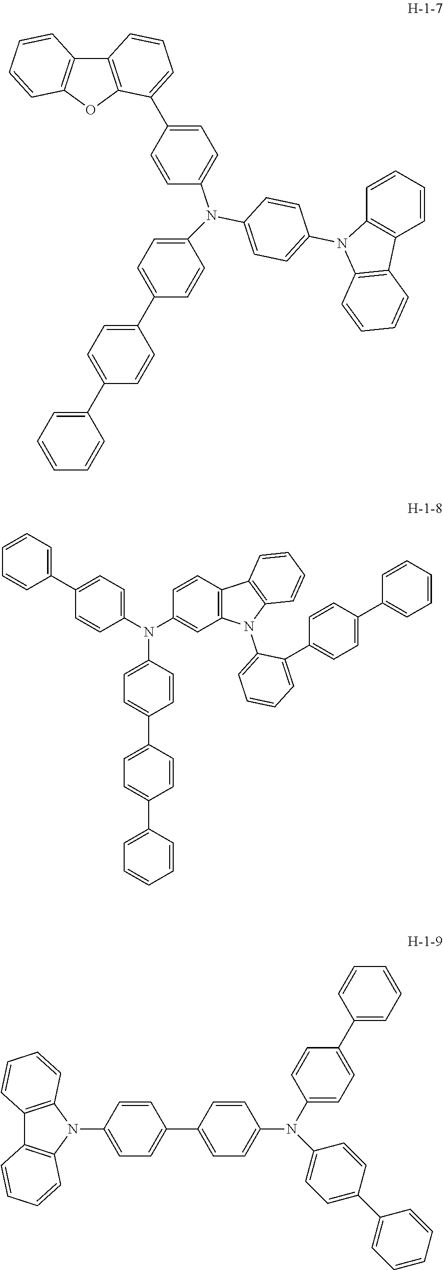 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

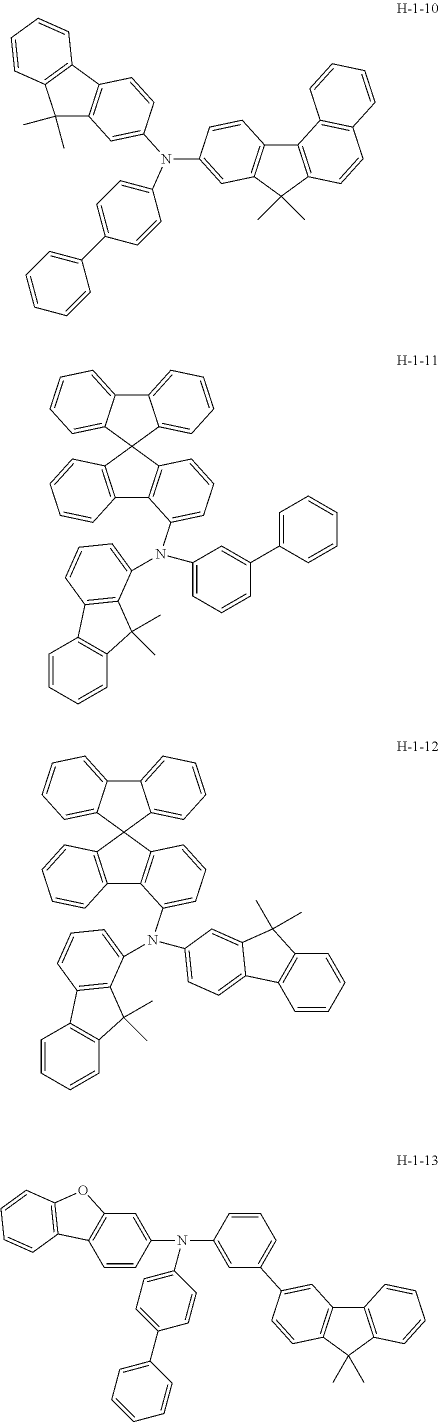 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

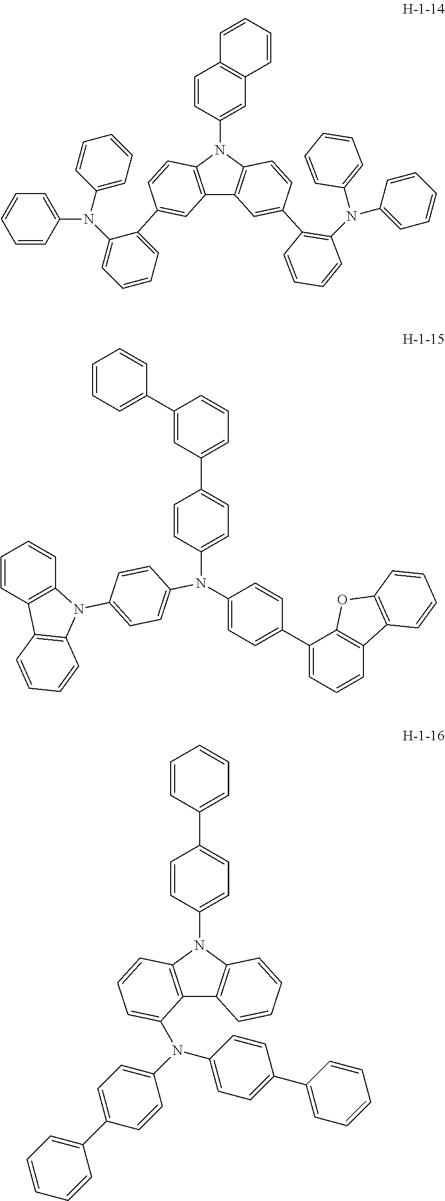 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

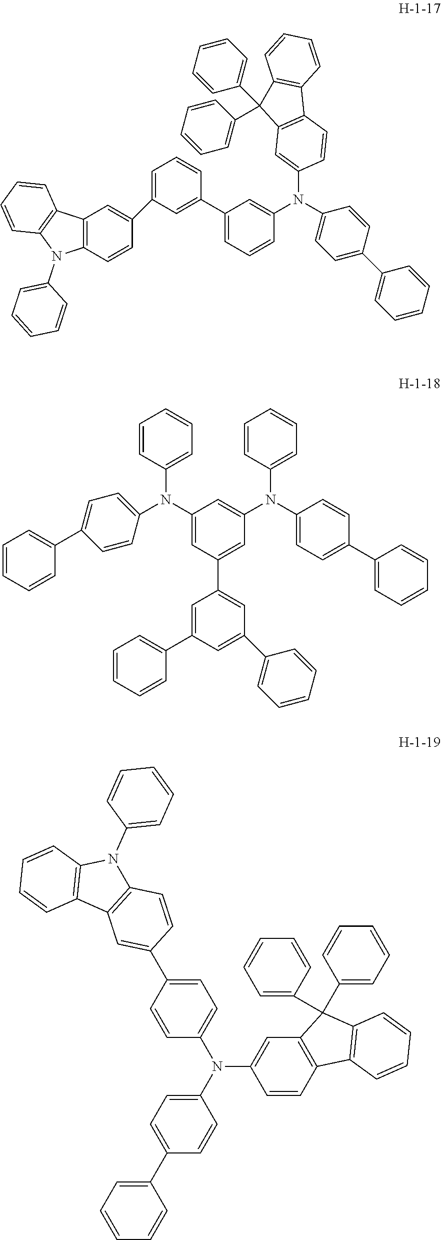 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine; N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine] (m-MTDATA), 4,4′,4″-tris(N,N-diphenylamino)triphenylamine (TDATA), 4,4′,4″-tris{N,-(2-naphthyl)-N-phenylamino)-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HAT-CN), etc.

In some embodiments, the hole transport region HTR may include carbazole derivatives such as N-phenyl carbazole and polyvinyl carbazole, fluorene derivatives, N,N′-bis(3-methylphenyl)-N,N′-diphenyl-[1,1-biphenyl]-4,4′-diamine (TPD), triphenylamine derivatives such as 4,4′,4″-tris(N-carbazolyl)triphenylamine (TCTA), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 9-(4-tert-butylphenyl)-3,6-bis(triphenylsilyl)-9H-carbazole (CzSi), 9-phenyl-9H-3,9′-bicarbazole (CCP), 1,3-bis(N-carbazolyl)benzene (mCP), 1,3-bis(1,8-dimethyl-9H-carbazol-9-yl)benzene (mDCP), etc.

The hole transport region HTR may include the above-described compounds of the hole transport region in at least one of a hole injection layer HIL, a hole transport layer HTL, and/or an electron blocking layer EBL.

The thickness of the hole transport region HTR may be, for example, from about 50 Å to about 15,000 Å. The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, when the hole transport region HTR includes the electron blocking layer EBL, the electron blocking layer EBL may have a thickness of about 10 Å to about 1,000 Å. When the thicknesses of the hole transport region HTR, the hole injection layer HIL, the hole transport layer HTL and the electron blocking layer EBL satisfy the above-described ranges, satisfactory hole transport properties may be achieved without a substantial increase in driving voltage.

The hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but the embodiment of the present disclosure is not limited thereto. For example, the p-dopant may include a metal halide compound such as CuI or RbI, a quinone derivative such as tetracyanoquinodimethane (TCNQ) or 2,3,5,6-tetrafluoro-7,7′ 8,8-tetracyanoquinodimethane (F4-TCNQ), a metal oxide such as tungsten oxide or molybdenum oxide, a cyano group-containing compound such as dipyrazino[2,3-f: 2′,3′-h] quinoxaline-2,3,6,7,10,11-hexacarbonitrile (HAT-CN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but the embodiment of the present disclosure is not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be included in the hole transport region HTR may be utilized as a material to be included in the buffer layer. The electron blocking layer EBL is a layer that serves to prevent or reduce the electron injection from the electron transport region ETR to the hole transport region HTR.

In each of the light emitting elements ED of embodiments illustrated in FIGS. 3 to 6, the electron transport region ETR is provided on the emission layer EML. The electron transport region ETR may include at least one of the hole blocking layer HBL, the electron transport layer ETL, or the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the emission layer EML, but the embodiment of the present disclosure is not limited thereto. The electron transport region ETR may have a thickness, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1:

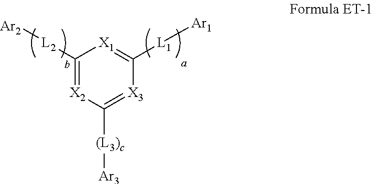

In Formula ET-1, at least one among X₁ to X₃ may be N, and the rest may each independently be CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer of 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAIq), berylliumbis(benzoquinolin-10-olate (Bebq2), 9,10-di(naphthalene-2-yl)anthracene (ADN), 1,3-bis[3,5-di(pyridin-3-yl)phenyl]benzene (BmPyPhB), or a mixture thereof.

In some embodiments, the electron transport region ETR may include a metal halide (such as LiF, NaCl, CsF, RbCI, RbI, CuI, and/or KI), a lanthanide metal (such as Yb), and/or a co-deposited material of the metal halide and the lanthanide metal. For example, the electron transport region ETR may include KI:Yb, RbI:Yb, LiF:Yb, etc., as a co-deposited material. In some embodiments, the electron transport region ETR may be formed utilizing a metal oxide such as Li₂O or BaO, or 8-hydroxyl-lithium quinolate (Liq), etc., but the embodiment of the present disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, or a metal stearate. The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described material(s), but the embodiment of the present disclosure is not limited thereto.

The electron transport region ETR may include the above-described compound(s) of the hole transport region in at least one of the electron injection layer EIL, the electron transport layer ETL, and/or the hole blocking layer HBL.

When the electron transport region ETR includes the electron transport layer ETL, the electron transport layer ETL may have a thickness of about 100 Å to about 1,000 Å, for example, about 150 Å to about 500 Å. When the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described range, satisfactory electron injection characteristics may be obtained without a substantial increase in driving voltage.

The second electrode EL2 may be provided on the electron transport region ETR. The second electrode EL2 may be a common electrode. The second electrode EL2 may be a cathode or an anode, but the embodiment of the present disclosure is not limited thereto. For example, when the first electrode EL1 is an anode, the second electrode EL2 may be a cathode, and when the first electrode EL1 is a cathode, the second electrode EL2 may be an anode. The second electrode EL2 may include at least one selected from among Ag, Mg, Cu, Al, Pt, Pd, Au, Ni, Nd, Ir, Cr, Li, Ca, LiF, Mo, Ti, W, In, Sn, and/or Zn, a compound of two or more selected from among these, a mixture of two or more selected from among these, or an oxide thereof.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/AI, Mo, Ti, Yb, W, or a compound or mixture thereof (e.g., AgMg, AgYb, or MgYb). In some embodiments, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal material(s), oxide(s) of the above-described metal material(s), and/or the like.

In some embodiments, the second electrode EL2 may be connected with an auxiliary electrode. When the second electrode EL2 is connected with the auxiliary electrode, the resistance of the second electrode EL2 may decrease.

In some embodiments, a capping layer CPL may further be disposed on the second electrode EL2 of the light emitting element ED of an embodiment. The capping layer CPL may include a multilayer or a single layer.

In an embodiment, the capping layer CPL may be an organic layer or an inorganic layer. For example, when the capping layer CPL contains an inorganic material, the inorganic material may include an alkaline metal compound (e.g., LiF), an alkaline earth metal compound (e.g., MgF₂), SiON, SiNx, SiOy, etc.

For example, when the capping layer CPL includes an organic material, the organic material may include a-NPD, NPB, TPD, m-MTDATA, Alq3, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., or an epoxy resin, or acrylate such as methacrylate. However, the embodiment of the present disclosure is not limited thereto, and the capping layer CPL may include at least one among Compounds P1 to P5:

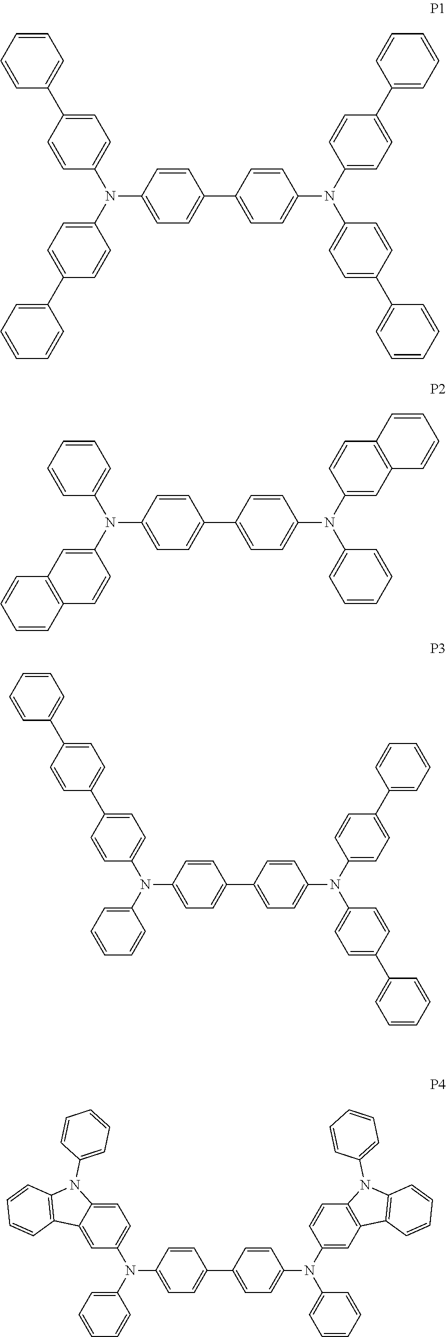 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

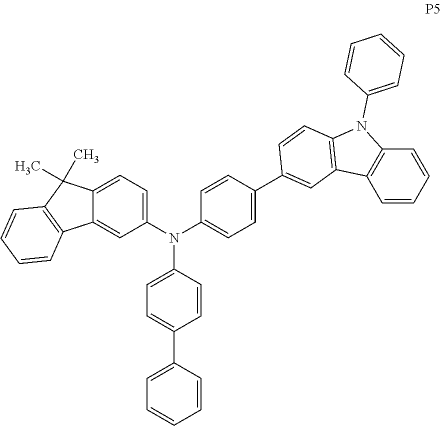 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be 1.6 or more with respect to light in a wavelength range of about 550 nm to about 660 nm.

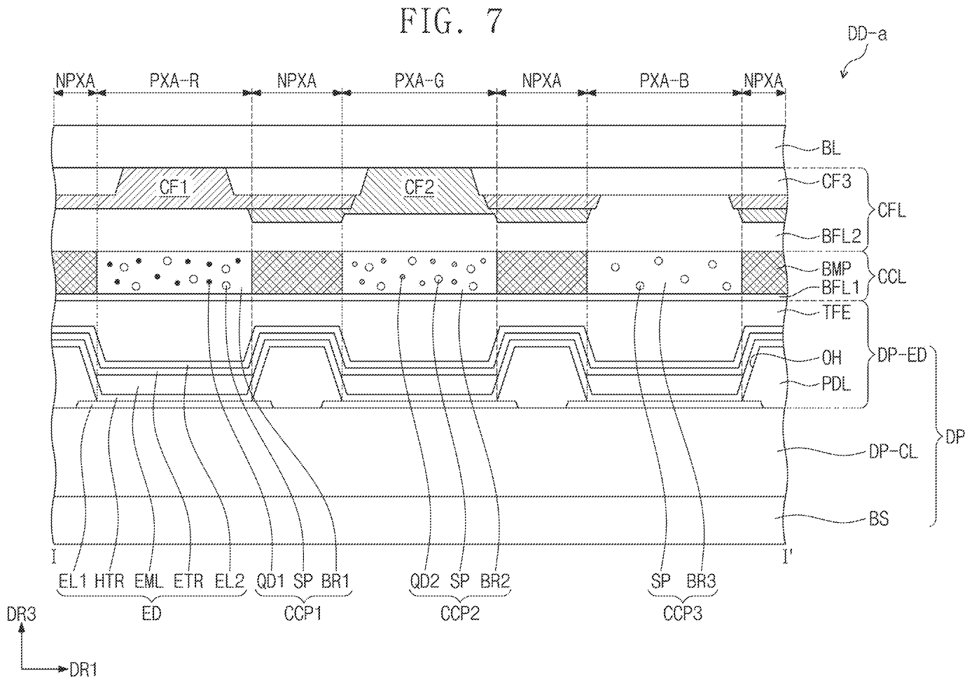

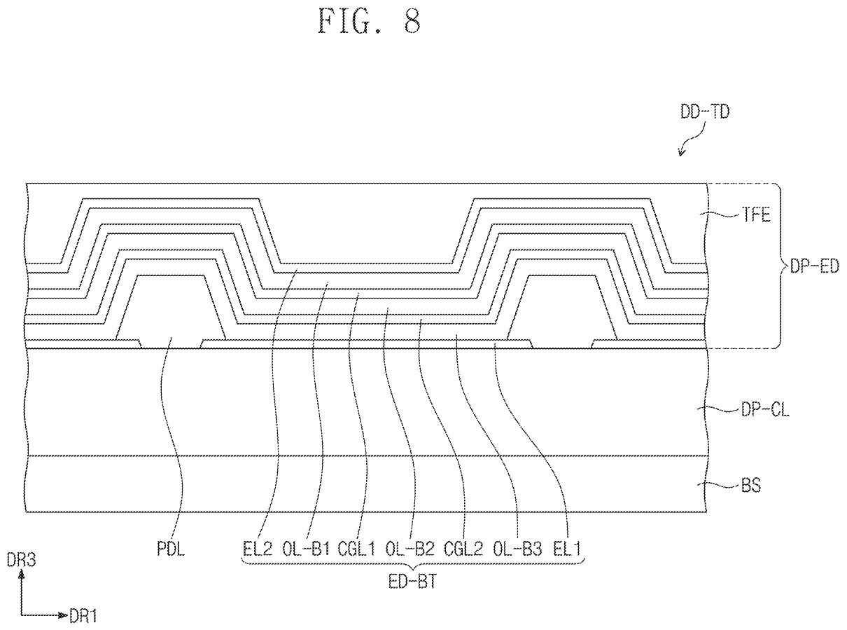

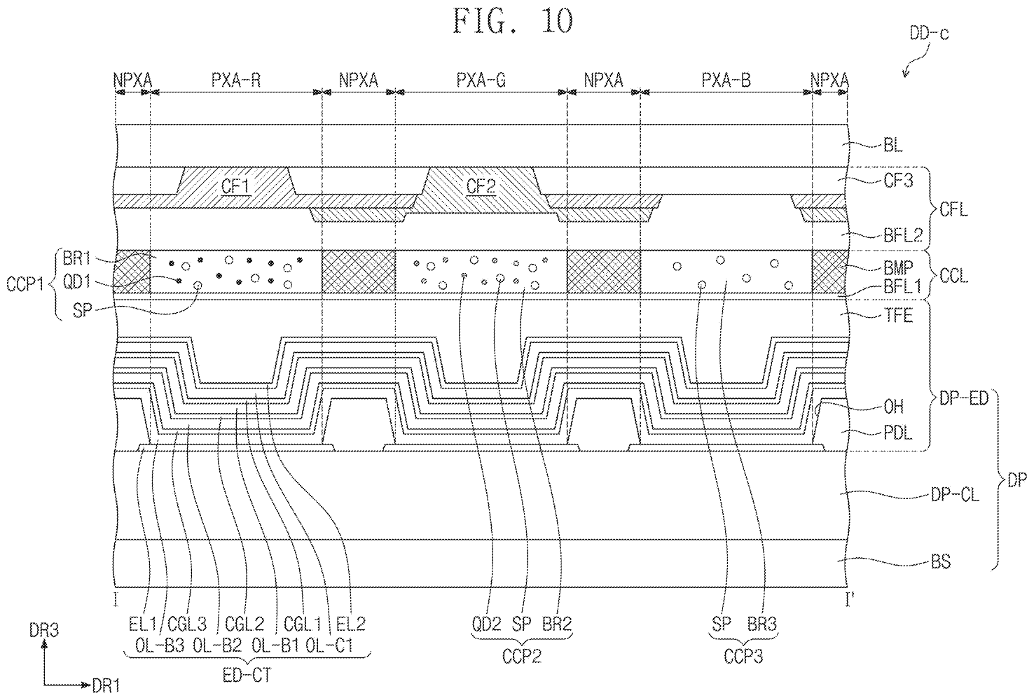

Each of FIGS. 7 to 10 is a cross-sectional view of a display device according to an embodiment of the present disclosure. Hereinafter, in describing the display devices of embodiments with reference to FIGS. 7 to 10, the duplicated features which have been described in FIGS. 1 to 6 are not described again, but their differences may be mainly described.

Referring to FIG. 7, the display device DD-a according to an embodiment may include a display panel DP including a display element layer DP-ED, a light control layer CCL disposed on the display panel DP, and a color filter layer CFL.

In an embodiment illustrated in FIG. 7, the display panel DP may include a base layer BS, a circuit layer DP-CL provided on the base layer BS, and the display element layer DP-ED, and the display element layer DP-ED may include a light emitting element ED.

The light emitting element ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. The light emitting element ED may include the nitrogen-containing compound of an embodiment. The structures of the light emitting elements of FIGS. 3 to 6 as described above may be equally applied to the structure of the light emitting element ED illustrated in FIG. 7.

Referring to FIG. 7, the emission layer EML may be disposed in an opening OH defined in a pixel defining film PDL. For example, the emission layer EML which is divided by the pixel defining film PDL and provided corresponding to each light emitting regions PXA-R, PXA-G, and PXA-B may be configured to emit light in substantially the same wavelength range. In the display device DD-a of an embodiment, the emission layer EML may be configured to emit blue light. In some embodiments, unlike the configuration illustrated, in an embodiment, the emission layer EML may be provided as a common layer in the entire light emitting regions PXA-R, PXA-G, and PXA-B.

The light control layer CCL may be disposed on the display panel DP. The light control layer CCL may include a light conversion body. The light conversion body may be a quantum dot, a phosphor, and/or the like. The light conversion body may be configured to emit provided light by converting the wavelength thereof. For example, the light control layer CCL may a layer containing the quantum dot or a layer containing the phosphor.

The light control layer CCL may include a plurality of light control parts CCP1, CCP2 and CCP3. The light control parts CCP1, CCP2, and CCP3 may be spaced apart from each other.

Referring to FIG. 7, divided patterns BMP may be disposed between the light control parts CCP1, CCP2 and CCP3 which are spaced apart from each other, but the embodiment of the present disclosure is not limited thereto. FIG. 7 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same as described above may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, and/or hollow sphere silica. The scatterer SP may include any one among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block or reduce the light control parts CCP1, CCP2 and CCP3 from being exposed to moisture/oxygen. In some embodiments, the barrier layer BFL1 may cover the light control parts CCP1, CCP2, and CCP3. In some embodiments, the barrier layer BFL2 may be provided between the light control parts CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In some embodiments, the barrier layers BFL1 and BFL2 may further include an organic film. The barrier layers BFL1 and BFL2 may be formed of a single layer or a plurality of layers.

In the display device DD-a of an embodiment, the color filter layer CFL may be disposed on the light control layer CCL. For example, the color filter layer CFL may be directly disposed on the light control layer CCL. In this case, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or dye. The first filter CF1 may include a red pigment or dye, the second filter CF2 may include a green pigment or dye, and the third filter CF3 may include a blue pigment or dye. In some embodiments, the embodiment of the present disclosure is not limited thereto, and the third filter CF3 may not include (e.g., may exclude) a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

Furthermore, in an embodiment, the first filter CF1 and the second filter CF2 may be a yellow filter. The first filter CF1 and the second filter CF2 may not be separated but be provided as one filter. The first to third filters CF1, CF2, and CF3 may be disposed corresponding to the red light emitting region PXA-R, the green light emitting region PXA-G, and the blue light emitting region PXA-B, respectively.

Although not illustrated, the color filter layer CFL may further include a light shielding part. The light shielding part may be a black matrix. The light shielding part may include an organic light shielding material or an inorganic light shielding material containing a black pigment or dye. The light shielding part may prevent or reduce light leakage, and may separate boundaries between the adjacent filters CF1, CF2, and CF3.

A base substrate BL may be disposed on the color filter layer CFL. The base substrate BL may be a member which provides a base surface in which the color filter layer CFL, the light control layer CCL, and/or the like are disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, unlike the configuration illustrated, in an embodiment, the base substrate BL may not be provided.

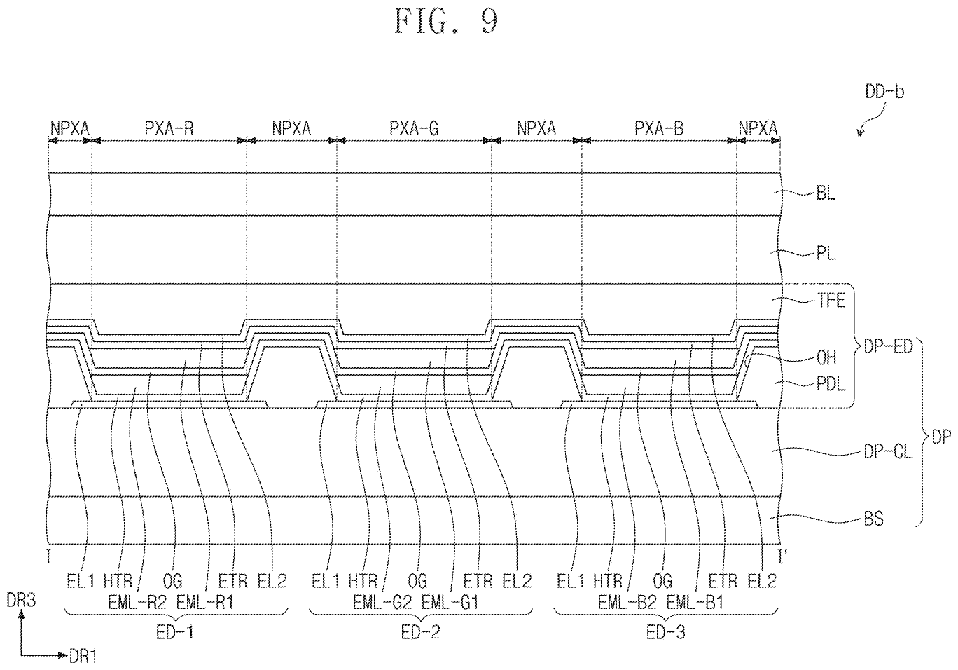

FIG. 8 is a cross-sectional view illustrating a portion of a display device according to an embodiment. FIG. 8 illustrates another embodiment of a part corresponding to the display panel DP of FIG. 7. In the display device DD-TD of an embodiment, the light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. At least one among the plurality of light emitting structures OL-B1, OL-B2, and OL-B3 may include the nitrogen-containing compound of an embodiment.

The light emitting structures OL-B1, OL-B2, and OL-B3 each may include an emission layer EML (FIG. 7) and a hole transport region HTR and an electron transport region ETR disposed with the emission layer EML (FIG. 7) located therebetween. For example, the light emitting element ED-BT included in the display device DD-TD of an embodiment may be a light emitting element having a tandem structure and including a plurality of emission layers.

In an embodiment illustrated in FIG. 8, all light beams respectively emitted from the light emitting structures OL-B1, OL-B2, and OL-B3 may be blue light. However, the embodiment of the present disclosure is not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may be configured to emit white light.

Charge generation layers CGL1 and CGL2 may be respectively disposed between two of the neighboring light emitting structures OL-B1, OL-B2, and OL-B3. The charge generation layers CGL1 and CGL2 may include a p-type charge (p-charge) generation layer and/or an n-type charge (n-charge) generation layer.

Referring to FIG. 9, the display device DD-b according to an embodiment may include light emitting elements ED-1, ED-2, and ED-3 in which two emission layers are stacked. Compared with the display device DD of an embodiment illustrated in FIG. 2, an embodiment illustrated in FIG. 9 has a difference in that the first to third light emitting elements ED-1, ED-2, and ED-3 each include two emission layers stacked in the thickness direction. In each of the first to third light emitting elements ED-1, ED-2, and ED-3, the two emission layers may be configured to emit light in substantially the same wavelength region. At least one among the first to third light emitting elements ED-1, ED-2, and ED-3 may include the nitrogen-containing compound of an embodiment. For example, the third light emitting element ED-3 may include the nitrogen-containing compound of an embodiment.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. More specifically, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, the embodiment of the present disclosure is not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be disposed between the emission auxiliary part OG and the electron transport region ETR. The second red emission layer EML-R2, the second green emission layer EML-G2, and the second blue emission layer EML-B2 may be disposed between the hole transport region HTR and the emission auxiliary part OG.

For example, the first light emitting element ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked. The second light emitting element ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting element ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

In some embodiments, an optical auxiliary layer PL may be disposed on the display element layer DP-ED. The optical auxiliary layer PL may include a polarizing layer. The optical auxiliary layer PL may be disposed on the display panel DP and control reflected light in the display panel DP due to external light. Unlike the configuration illustrated, the optical auxiliary layer PL in the display device according to an embodiment may not be provided.

Unlike FIGS. 8 and 9, FIG. 10 illustrates that a display device DD-c includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. At least one among the first to fourth light emitting structures OL-B1, OL-B2, OL-B3, and OL-C1 may include the nitrogen-containing compound of an embodiment.

Among the four light emitting structures, the first to third light emitting structures OL-B1, OL-B2, and OL-B3 may be configured to emit blue light, and the fourth light emitting structure OL-C1 may be configured to emit green light. However, the embodiment of the present disclosure is not limited thereto, and the first to fourth light emitting structures OL-B1, OL-B2, OL-B3, and OL-C1 may be configured to emit light beams in different wavelength regions.

Charge generation layers CGL1, CGL2, and CGL3 may be disposed between the first to fourth light emitting structures OL-B1, OL-B2, OL-B3, and OL-C1. The charge generation layers CGL1, CGL2, and CGL3 disposed between adjacent light emitting structures OL-B1, OL-B2, OL-B3, and OL-C1 may include a p-type or kind charge generation layer and/or an n-type or kind charge generation layer.

Hereinafter, with reference to Examples and Comparative Examples, a nitrogen-containing compound according to an embodiment of the present disclosure and a light emitting element of an embodiment will be described in more detail. In some embodiments, Examples described are only illustrations to assist the understanding of the present disclosure, and the scope of the present disclosure is not limited thereto.

### EXAMPLES

### 1. Synthesis of Nitrogen-Containing Compound of Example

First, a synthetic method of a nitrogen-containing compound according to the current embodiment will be described in more detail by illustrating synthetic methods of Compounds 1, 22, 29, and 35. In some embodiments, in the following descriptions, a synthetic method of the nitrogen-containing compound is provided as an example, but an embodiment of the present disclosure is not limited to the following examples.

### (1) Synthesis of Nitrogen-Containing Compound 1

Nitrogen-containing Compound 1 according to an example may be synthesized by, for example, the steps (tasks) shown in Reaction Scheme 1:

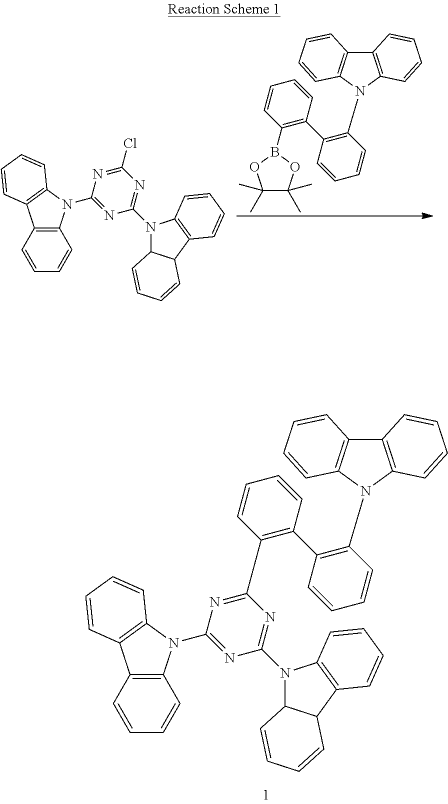

9-(2′-(4,4,5,5-tetramethyl-1,3,2-dioxaborolan-2-yl)-[1,1′-biphenyl]-2-yl)-9H-carbazole (2.39 g), 9,9′-(6-chloro-1,3,5-triazine-2,4-diyl)bis(9H-carbazole) (2 g), potassium carbonate (1.55 g), and tetrakis(triphenylphosphine)palladium(0) (0.2 g) were added to a round bottom flask (RB), dissolved in 28 mL of tetrahydrofuran (THF) and 7 mL of distilled water (DW), and refluxed for about 12 hours. After the reaction was completed, the reaction solution was extracted with ethyl acetate to collect organic layers. The organic layers were dried over magnesium sulfate to obtain residues. The residues were separated and purified by silica gel column chromatography to obtain Compound 1 (1.79 g, yield: 55%). Compound 1 above was identified by LC-MS.

C₅₁H₃₂N₆ M+1: 729.33

(2) Synthesis of Nitrogen-Containing Compound 22

Nitrogen-containing Compound 22 according to an example may be synthesized by, for example, the steps shown in Reaction Scheme 2:

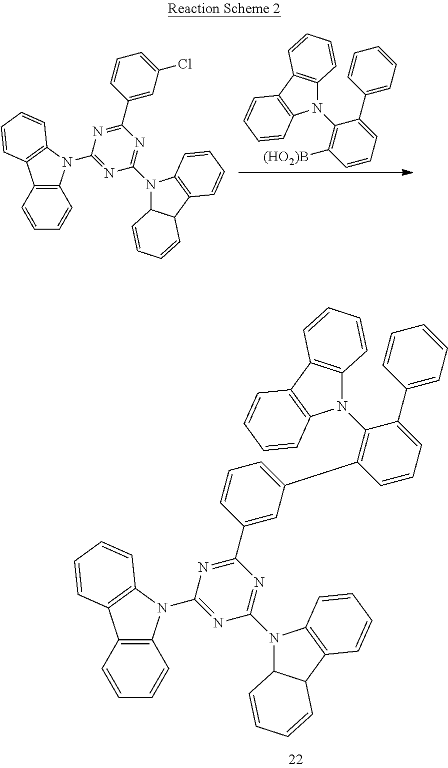

2-(9H-carbazol-9-yl)-[1,1′-biphenyl]-3-yl)boronic acid (1.67 g), 9,9′-(6-(3-chlorophenyl)-1,3,5-triazine-2,4-diyl)bis(9H-carbazole) (2 g), potassium carbonate (1.33 g), and tetrakis(triphenylphosphine)palladium(0) (0.17 g) were added to a RB, dissolved in 24 mL of THF and 6 mL of DW, and refluxed for about 12 hours. After the reaction was completed, the reaction solution was extracted with ethyl acetate to collect organic layers. The organic layers were dried over magnesium sulfate to obtain residues. The residues were separated and purified by silica gel column chromatography to obtain Compound 22 (2.18 g, yield: 71%). Compound 22 above was identified by LC-MS.

C₅₇H₃₆N₆ M+1: 805.51

(3) Synthesis of Nitrogen-Containing Compound 29

Nitrogen-containing Compound 29 according to an example may be synthesized by, for example, the steps shown in Reaction Scheme 3:

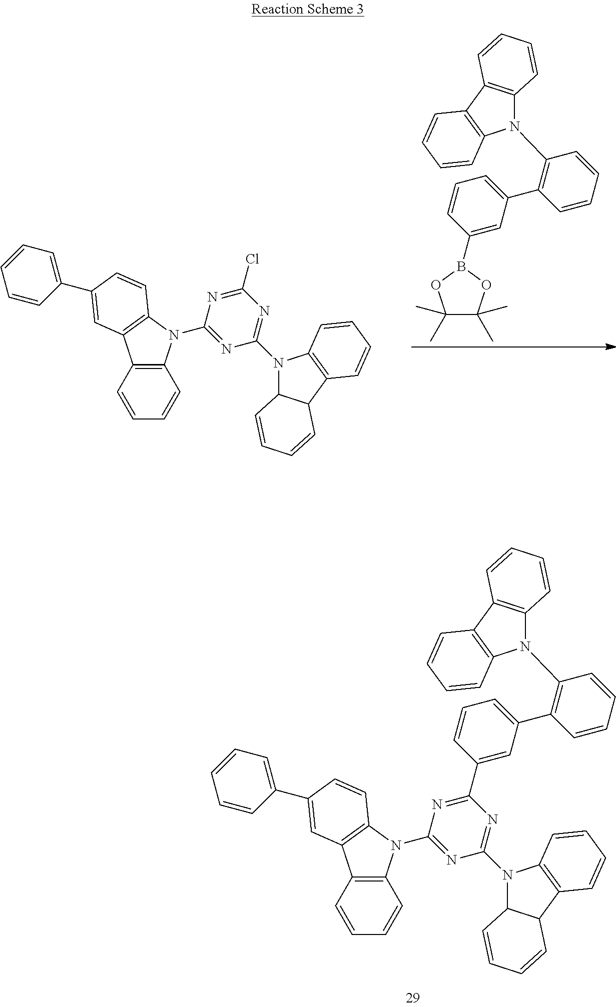 [SMILES: Clc1nc(N2c3ccccc3C3C=CC=CC32)nc(-n2c3ccccc3c3cc(-c4ccccc4)ccc32)n1 ; CC1(C)OB(c2cccc(-c3ccccc3-n3c4ccccc4c4ccccc43)c2)OC1(C)C ; C1=CC2c3ccccc3N(c3nc(-c4cccc(-c5ccccc5-n5c6ccccc6c6ccccc65)c4)nc(-n4c5ccccc5c5cc(-c6ccccc6)ccc54)n3)C2C=C1]

9-(3′-(4,4,5,5-tetramethyl-1,3,2-dioxaborolan-2-yl)-[1,1′-biphenyl]-2-yl)-9H-carbazole (1.33 g), 9-(4-(9H-carbazol-9-yl)-6-chloro-1,3,5-triazin-2-yl)-3-phenyl-9H-carbazole (1.3 g), potassium carbonate (0.86 g), and tetrakis(triphenylphosphine)palladium(0) (0.11 g) were added to a RB, dissolved in 20 mL of THF and 5 mL of DW, and refluxed for about 12 hours. After the reaction was completed, the reaction solution was extracted with ethyl acetate to collect organic layers. The organic layers were dried over magnesium sulfate to obtain residues. The residues were separated and purified by silica gel column chromatography to obtain Compound 29 (1.6 g, yield: 80%). Compound 29 above was identified by LC-MS.

C₅₇H₃₆N₆ M+1: 805.44

(4) Synthesis of Nitrogen-Containing Compound 35

Nitrogen-containing Compound 35 according to an example may be synthesized by, for example, the steps shown in Reaction Scheme 4:

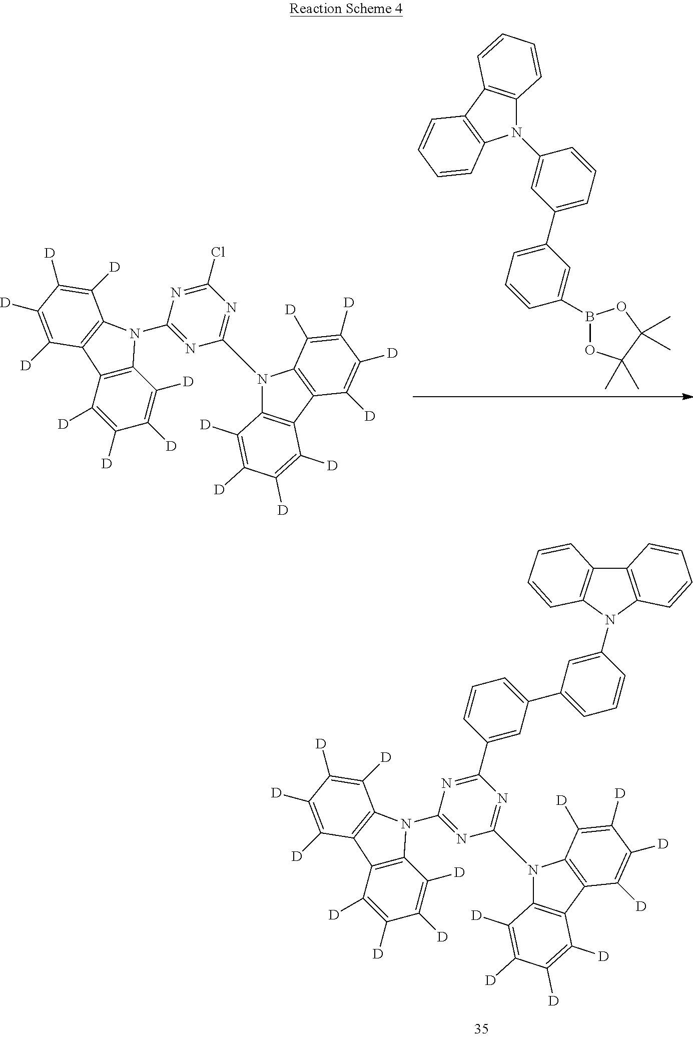 [SMILES: [2H]c1c([2H])c([2H])c2c(c1[2H])c1c([2H])c([2H])c([2H])c([2H])c1n2-c1nc(Cl)nc(-n2c3c([2H])c([2H])c([2H])c([2H])c3c3c([2H])c([2H])c([2H])c([2H])c32)n1 ; CC1(C)OB(c2cccc(-c3cccc(-n4c5ccccc5c5ccccc54)c3)c2)OC1(C)C ; [2H]c1c([2H])c([2H])c2c(c1[2H])c1c([2H])c([2H])c([2H])c([2H])c1n2-c1nc(-c2cccc(-c3cccc(-n4c5ccccc5c5ccccc54)c3)c2)nc(-n2c3c([2H])c([2H])c([2H])c([2H])c3c3c([2H])c([2H])c([2H])c([2H])c32)n1]

9-(3′-(4,4,5,5-tetramethyl-1,3,2-dioxaborolan-2-yl)-[1,1′-biphenyl]-3-yl)-9H-carbazole (1.73 g), 9,9′-(6-chloro-1,3,5-triazine-2,4-diyl)bis(9H-carbazole-1,2,3,4,5,6,7,8-d8) (1.5 g), potassium carbonate (1.12 g), and tetrakis(triphenylphosphine)palladium(0) (0.15 g) were added to a RB, dissolved in 20 mL of THF and 5 mL of DW, and refluxed for about 12 hours. After the reaction was completed, the reaction solution was extracted with ethyl acetate to collect organic layers. The organic layers were dried over magnesium sulfate to obtain residues. The residues were separated and purified by silica gel column chromatography to obtain Compound 35 (2.05 g, yield: 85%). Compound 35 above was identified by LC-MS.

C₅₁H₁₆D₁₆N₆ M+1: 745.62

### 2. Manufacture and Evaluation of Light Emitting Elements

### (1) Manufacture of Light Emitting Elements

Light emitting elements including the nitrogen-containing compounds of examples or Comparative Example Compound in the emission layers were manufactured as follows. Compounds 1, 22, 29, and 35 that are the nitrogen-containing compounds of examples were utilized as a host material for the emission layer to manufacture the light emitting elements of Examples 1 to 4, respectively. Comparative Example Compound CX1 to CX5 were utilized as a host material to manufacture the light emitting elements of Comparative Examples 1 to 5.

For a first electrode, an ITO glass substrate of about 15 Ω/cm² (about 1,200 Å) made by Corning Co. was cut to a size of 50 mm×50 mm×0.7 mm, cleansed by ultrasonic waves utilizing isopropyl alcohol and pure water for about 5 minutes each, and then irradiated with ultraviolet rays for about 30 minutes and exposed to ozone and cleansed. The glass substrate was installed on a vacuum deposition apparatus. On the first electrode, N,N′-di(naphthalen-1-yl)-N,N′-diphenyl-benzidine (NPB), which is a suitable compound, was deposited in vacuum to form a 300 Å-thick hole injection layer, and then mCP was deposited in vacuum to form a 200 Å-thick hole transport layer. Then, a host and a dopant were co-deposited in a weight ratio of 92:8 to form a 250 Å-thick emission layer. The nitrogen-containing compound of an example or Comparative Example Compound was utilized as the host, and Firpic was utilized as the dopant.

TAZ was provided to form a 200 Å-thick electron transport layer. LiF, which is an alkaline metal halide, was deposited on the electron transport layer to a 10 Å-thick electron injection layer, and Al was deposited in vacuum to form a 100 Å-thick LiF/AI electrode (negative electrode), thereby manufacturing a light emitting element.

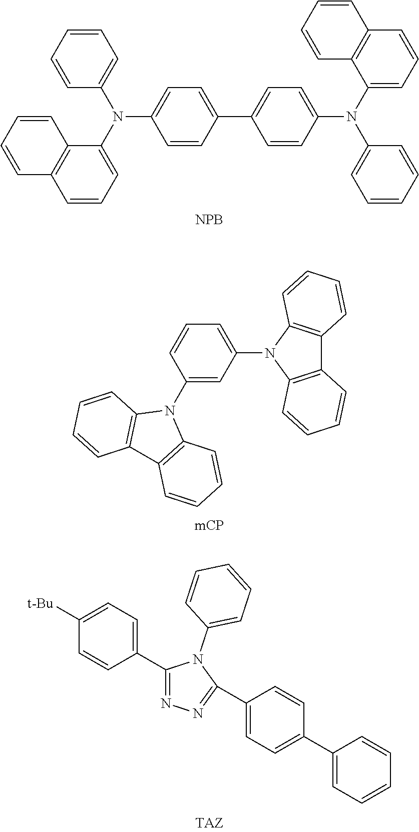 [SMILES: c1ccc(N(c2ccc(-c3ccc(N(c4ccccc4)c4cccc5ccccc45)cc3)cc2)c2cccc3ccccc23)cc1 ; "NPB" ; c1cc(-n2c3ccccc3c3ccccc32)cc(-n2c3ccccc3c3ccccc32)c1 ; CC(C)(C)c1ccc(-c2nnc(-c3ccc(-c4ccccc4)cc3)n2-c2ccccc2)cc1]

Example Compounds and Comparative Example Compounds utilized in Examples 1 to 4 and Comparative Examples 1 to 5 are listed in Table 1.

TABLE 1

| | |
|---|---|
| Compound 1 | 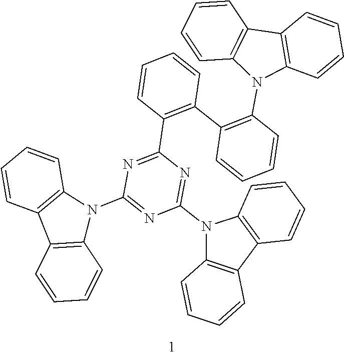 [SMILES: c1ccc(-c2ccccc2-n2c3ccccc3c3ccccc32)c(-c2nc(-n3c4ccccc4c4ccccc43)nc(-n3c4ccccc4c4ccccc43)n2)c1] |
| Compound 22 | 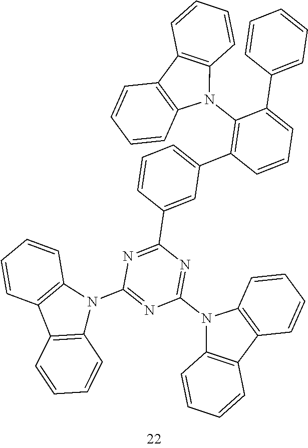 [SMILES: c1ccc(-c2cccc(-c3cccc(-c4nc(-n5c6ccccc6c6ccccc65)nc(-n5c6ccccc6c6ccccc65)n4)c3)c2-n2c3ccccc3c3ccccc32)cc1] |
| Compound 29 | 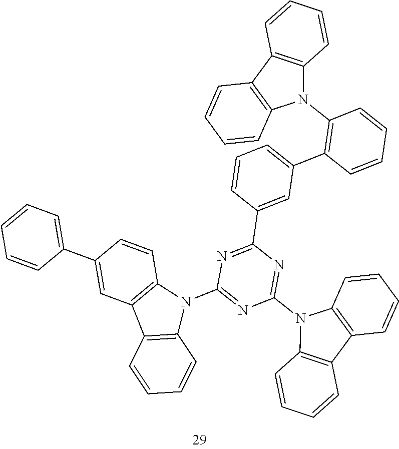 [SMILES: c1ccc(-c2ccc3c(c2)c2ccccc2n3-c2nc(-c3cccc(-c4ccccc4-n4c5ccccc5c5ccccc54)c3)nc(-n3c4ccccc4c4ccccc43)n2)cc1] |
| Compound 35 | 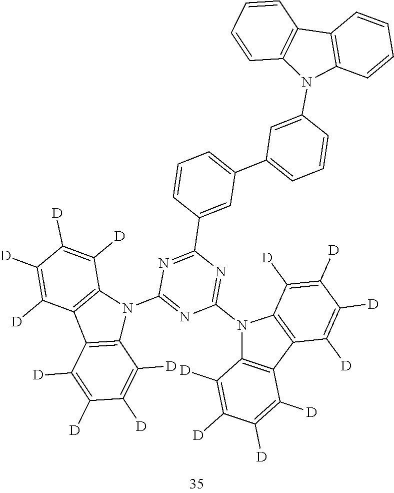 [SMILES: [2H]c1c([2H])c([2H])c2c(c1[2H])c1c([2H])c([2H])c([2H])c([2H])c1n2-c1nc(-c2cccc(-c3cccc(-n4c5ccccc5c5ccccc54)c3)c2)nc(-n2c3c([2H])c([2H])c([2H])c([2H])c3c3c([2H])c([2H])c([2H])c([2H])c32)n1] |
| Comparative Example Compound CX1 | 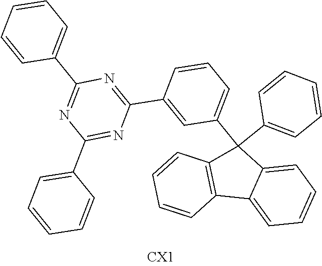 [SMILES: c1ccc(-c2nc(-c3ccccc3)nc(-c3cccc(C4(c5ccccc5)c5ccccc5-c5ccccc54)c3)n2)cc1] |
| Comparative Example Compound CX2 | 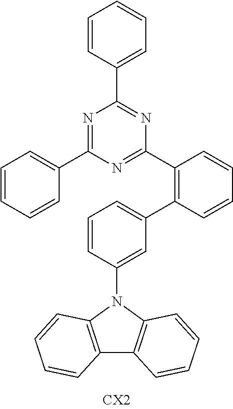 [SMILES: c1ccc(-c2nc(-c3ccccc3)nc(-c3ccccc3-c3cccc(-n4c5ccccc5c5ccccc54)c3)n2)cc1] |
| Comparative Example Compound CX3 | 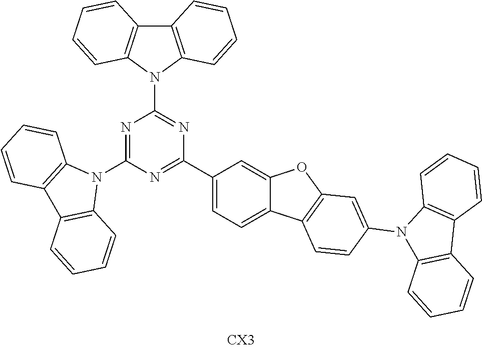 [SMILES: c1ccc2c(c1)c1ccccc1n2-c1ccc2c(c1)oc1cc(-c3nc(-n4c5ccccc5c5ccccc54)nc(-n4c5ccccc5c5ccccc54)n3)ccc12] |
| Comparative Example Compound CX4 | 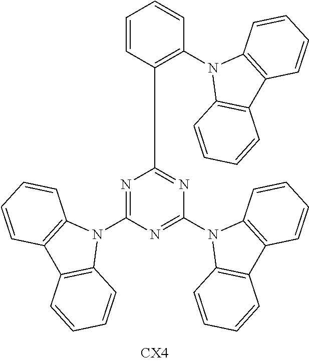 [SMILES: c1ccc(-n2c3ccccc3c3ccccc32)c(CCc2nc(-n3c4ccccc4c4ccccc43)nc(-n3c4ccccc4c4ccccc43)n2)c1] |
| Comparative Example Compound CX5 | 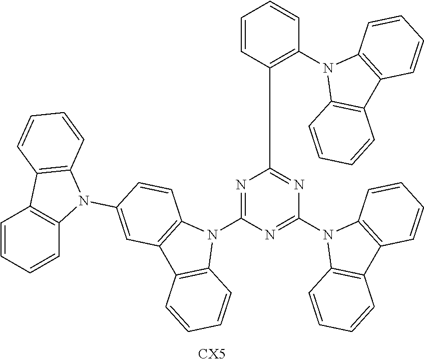 [SMILES: c1ccc(-n2c3ccccc3c3ccccc32)c(CCc2nc(-n3c4ccccc4c4ccccc43)nc(-n3c4ccccc4c4cc(-n5c6ccccc6c6ccccc65)ccc43)n2)c1] |
| — | — |

### (2) Evaluation of Light Emitting Element Characteristics

In each of the light emitting elements of Examples and Comparative Examples, the luminous efficiency and maximum quantum efficiency were evaluated, and the lowest triplet excitation energy level (T1 level) of each of Example Compounds and Comparative Example Compounds was evaluated, and the results are listed in Table 2. The driving voltage and maximum quantum efficiency were evaluated based on a current density of 10 mA/cm². The driving voltage was measured by utilizing SourceMeter (Keithley Instruments, Inc., 2400 series), and the maximum quantum efficiency was measured by utilizing an external quantum efficiency measurement apparatus, C9920-2-12 manufactured by Hamamatsu Photonics, Co., Japan. With respect to the evaluation of maximum quantum efficiency, brightness/current density was measured by utilizing brightness photometer in which wavelength sensitivity is calibrated, and the maximum quantum efficiency is converted assuming angular brightness distribution (Lambertian distribution) in which ideal diffuse reflecting surface is contemplated.

The luminous efficiency was measured based on a brightness of 1,000 cd/in². The value of the lowest triplet excitation energy level (Ti level) was calculated by a non-empirical molecular orbital method, and specifically, was calculated with B3LYP/6-31 G(d) utilizing Gaussian 09 from Gaussian, Inc. (Wallingford, CT, USA).

TABLE 2

| | | | | Maximum | | |
|---|---|---|---|---|---|---|
| | | Driving | | quantum | T1 | |
| | | voltage | Efficiency | efficiency | level | Emission |
| Division | Emission layer | (V) | (Cd/A) | (%) | (eV) | color |
| Example 1 | Compound 1 | 4.0 | 21.7 | 27.3 | 3.00 | Blue |
| Example 2 | Compound 22 | 4.2 | 22.4 | 26.9 | 2.96 | Blue |
| Example 3 | Compound 29 | 4.2 | 23.5 | 28.1 | 2.97 | Blue |
| Example 4 | Compound 35 | 4.3 | 24.2 | 28.8 | 2.93 | Blue |
| Comparative | Comparative | 4.9 | 15.2 | 26.2 | 2.89 | Blue |
| Example 1 | Example | | | | | |
| | Compound CX1 | | | | | |
| Comparative | Comparative | 4.5 | 18.7 | 26.1 | 2.89 | Blue |
| Example 2 | Example | | | | | |
| | Compound CX2 | | | | | |
| Comparative | Comparative | 4.8 | 19.2 | 24.3 | 2.83 | Blue |
| Example 3 | Example | | | | | |
| | Compound CX3 | | | | | |
| Comparative | Comparative | 4.7 | 20.9 | 26.1 | 2.90 | Blue |
| Example 4 | Example | | | | | |
| | Compound CX4 | | | | | |
| Comparative | Comparative | 4.7 | 19.1 | 25.0 | 2.90 | Blue |
| Example 5 | Example | | | | | |
| | Compound CX5 | | | | | |

Referring to Table 2, it may be seen that the light emitting elements of Examples 1 to 4 have a decrease in the driving voltage, and have an excellent or suitable light efficiency and maximum quantum efficiency compared to the light emitting elements of Comparative Examples 1 to 5. In some embodiments, it may be seen that Compounds 1, 22, 29, and 35 have relatively high lowest triplet excitation energy level compared to Comparative Example Compounds CX1 to CX5. The light emitting elements of Examples 1 to 4 include Compounds 1, 22, 29, and 35, and Compounds 1, 22, 29, and 35 include three carbazole groups bonded to the triazine group. In Compounds 1, 22, 29, and 35, two carbazole groups among the three carbazole groups are directly bonded to the triazine group, and the remaining one carbazole group is bonded via the biphenyl group. Accordingly, it is thought that the light emitting elements of Examples 1 to 4 including Compounds 1, 22, 29, and 35 have a decrease in the driving voltage and exhibit excellent or suitable light efficiency and maximum quantum efficiency. Compounds 1, 22, 29, and 35 are the nitrogen-containing compounds of examples. Accordingly, the light emitting element including the nitrogen-containing compound of an example may exhibit characteristics in which the driving voltage decreases, and the light efficiency and maximum quantum efficiency are excellent or suitable.

The light emitting element of Comparative Example 1 includes Comparative Example Compound CX1, and Comparative Example Compound CX1 includes a triazine group, a fluorenyl group bonded to the triazine group, and a phenyl group bonded to the triazine group. Referring to Table 2, it may be seen that Comparative Example Compound CX1 has relatively low lowest triplet excitation energy level. It may be seen that the light emitting element of Comparative Example 1 including Comparative Example Compound CX1 exhibits relatively high driving voltage and low efficiency. It is thought that Comparative Example Compound CX1 does not include a biphenyl group and a carbazole group, and thus exhibits relatively low lowest triplet excitation energy level, and the light emitting element of Comparative Example 1 exhibits relatively low efficiency.

The light emitting element of Comparative Example 2 includes Comparative Example Compound CX2, and Comparative Example Compound CX2 corresponds only one carbazole group. Accordingly, it is thought that Comparative Example Compound CX2 exhibits a relatively low value of the lowest triplet excitation energy level, and the light emitting element of Comparative Example 2 exhibits relatively low efficiency.

The light emitting elements of Comparative Examples 3 to 5 include Comparative Example Compounds CX3 to CX5, and Comparative Example Compounds CX3 to CX5 do not include a biphenyl group. Comparative Example Compound CX3 is that a carbazole group is bonded to the triazine group via a dibenzofuran group, and Comparative Example Compounds CX4 and CX5 are that a carbazole group is bonded to the triazine group via a phenyl group. Accordingly, it is thought that Comparative Example Compounds CX3 to CX5 exhibit a relatively low value of the lowest triplet excitation energy level, and the light emitting elements of Comparative Examples 3 to 5 exhibits relatively high driving voltages and relatively low efficiencies.

According to an embodiment, a light emitting element is provided to include a first electrode, a second electrode disposed on the first electrode, and an emission layer disposed between the first electrode and the second electrode. The emission layer may include the nitrogen-containing compound of an embodiment. The nitrogen-containing compound of an embodiment may include three carbazole groups which are bonded to a hexagonal monocyclic ring containing N as a ring-forming atom. Two carbazole groups among the three carbazole groups may be directly bonded to a hexagonal monocyclic ring containing N as a ring-forming atom, and the remaining one carbazole group may be bonded to the hexagonal monocyclic ring containing N as a ring-forming atom via a biphenyl group. Accordingly, the light emitting element including the nitrogen-containing compound of an embodiment may exhibit characteristics having a decrease in the driving voltage and improvement in the efficiency.

A light emitting element of an embodiment includes a nitrogen-containing compound of an embodiment, and thus the driving voltage may be reduced and the light efficiency may be improved.

The nitrogen-containing compound of an embodiment may contribute to reducing the driving voltage of the light emitting element, and improving the light efficiency thereof.

Although the present disclosure has been described with reference to a preferred embodiment of the present disclosure, it will be understood that the present disclosure should not be limited to these preferred embodiments but one or more suitable changes and modifications can be made by those skilled in the art without departing from the spirit and scope of the present disclosure.

Accordingly, the technical scope of the present disclosure is not intended to be limited to the contents set forth in the detailed description of the specification, but is intended to be defined by the appended claims and equivalents thereof.

What is claimed is:

1. A light emitting element comprising:
a first electrode;
a second electrode on the first electrode; and
an emission layer between the first electrode and the second electrode and comprising a first compound represented by Formula 1: 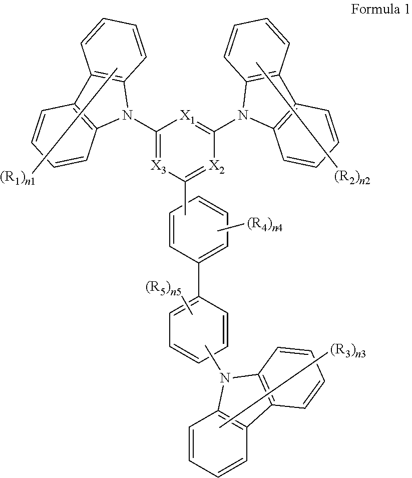
wherein, in Formula 1,
at least one among X₁ to X₃ is N, and the rest are each independently CR₆,
n1 to n3 are each independently an integer of 0 to 8,
n4 and n5 are each independently an integer of 0 to 4, and
R₁ to R₆ are each independently a hydrogen atom, a deuterium atom, a halogen atom, a hydroxy group, a cyano group, a nitro group, a substituted or unsubstituted amine group, a substituted or unsubstituted oxy group, a substituted or unsubstituted thio group, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted aryl group having 6 to 60 ring-forming carbon atoms, or a substituted or unsubstituted heteroaryl group having 2 to 60 ring-forming carbon atoms.

2. The light emitting element of claim 1, wherein Formula 1 is represented by Formula 1-1 or Formula 1-2: 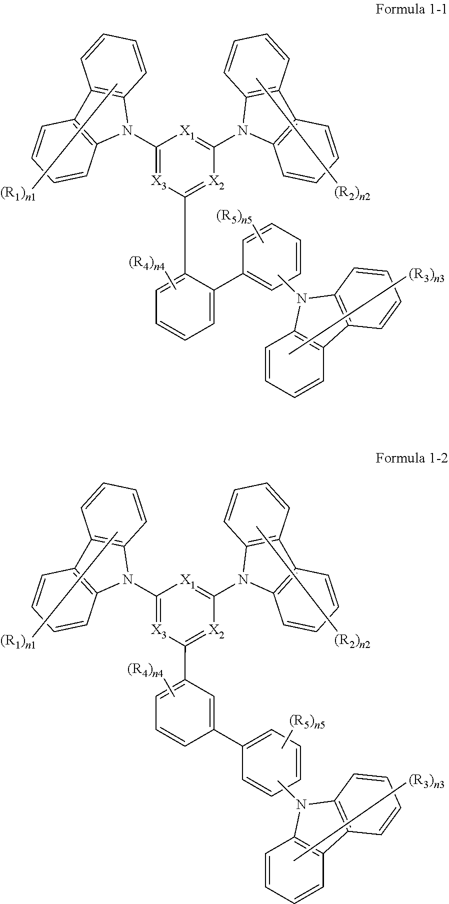
wherein, in Formula 1-1 and Formula 1-2,
X₁ to X₃, n1 to n5, and R₁ to R₅ are the same as defined in Formula 1.

3. The light emitting element of claim 2, wherein Formula 1-1 is represented by Formula 1-1A or Formula 1-1B: 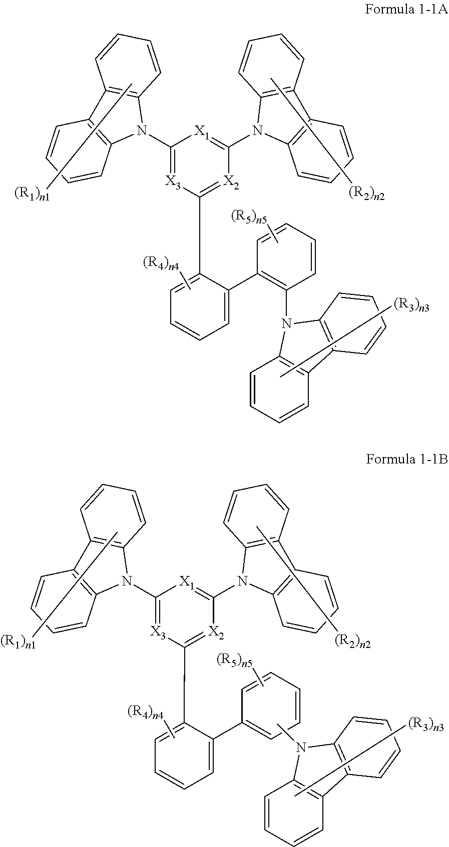
wherein, in Formula 1-1A and Formula 1-1B,
X₁ to X₃, n1 to n5, and R₁ to R₅ are the same as defined in Formula 1.

4. The light emitting element of claim 2, wherein Formula 1-2 is represented by Formula 1-2A or Formula 1-2B: 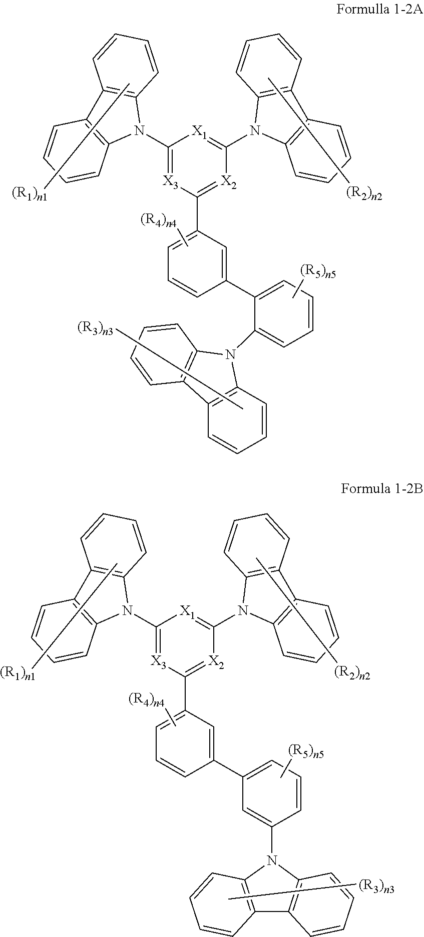
wherein, in Formula 1-2A and Formula 1-2B,
X₁ to X₃, n1 to n5, and R₁ to R₅ are the same as defined in Formula 1.

5. The light emitting element of claim 1, wherein Formula 1 is represented by any one among Formula 1-3 to Formula 1-5: 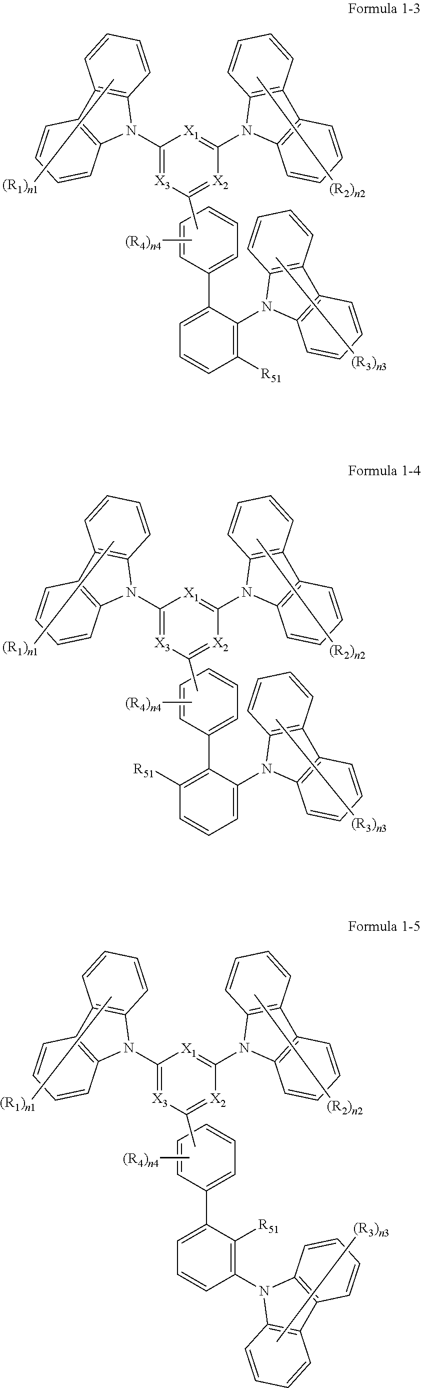
wherein, in Formula 1-3 to Formula 1-5,
R₅₁ is a substituted or unsubstituted phenyl group, a substituted or unsubstituted carbazole group, a substituted or unsubstituted dibenzofuran group, or a substituted or unsubstituted dibenzothiophene group, and
X₁ to X₃, n1 to n4, and R₁ to R₄ are the same as defined in Formula 1.

6. The light emitting element of claim 1, wherein, in Formula 1, R₁ to R₆ are each independently a hydrogen atom, a deuterium atom, or represented by any one among R-1 to R-7: 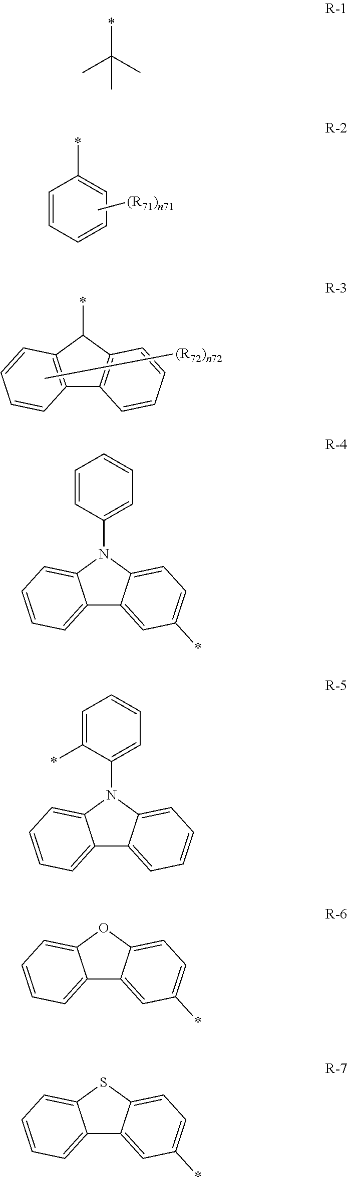
wherein, in R-2 and R-3,
n71 is an integer of 0 to 5,
n72 is an integer of 0 to 8, and
R₇₁ and R₇₂ are each independently a hydrogen atom, a deuterium atom, or an unsubstituted t-butyl group.

7. The light emitting element of claim 1, wherein, in Formula 1, at least one among R₁ to R₅ is a deuterium atom or comprises a substituent containing a deuterium atom.

8. The light emitting element of claim 1, wherein the emission layer comprises a host and a dopant, and
the host comprises the first compound.

9. The light emitting element of claim 8, wherein the host further comprises a second compound represented by Formula HT-1: 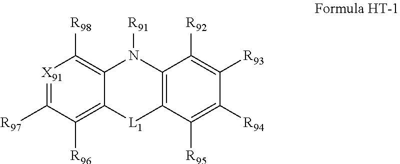
wherein, in Formula HT-1,
L₁ is a direct linkage, CR₉₉R₁₀₀, or SiR₁₀₁R₁₀₂,
X₉₁ is N or CR₁₀₃, and
R₉₁ to R₁₀₃ are each independently a hydrogen atom, a deuterium atom, a cyano group, a substituted or unsubstituted silyl group, a substituted or unsubstituted oxy group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, and/or are bonded to an adjacent group to form a ring.

10. The light emitting element of claim 8, wherein the dopant comprises a third compound represented by Formula M-a: 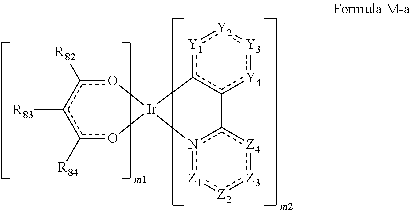
wherein, in Formula M-a,
Y₁ to Y₄ and Z₁ to Z₄ are each independently CR₈₁ or N,
R₈₁ to R₈₄ are each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or are bonded to an adjacent group to form a ring,
m1 is 0 or 1, and
m2 is 2 or 3.

11. The light emitting element of claim 1, wherein the first compound is represented by any one among compounds of Compound Group 1: 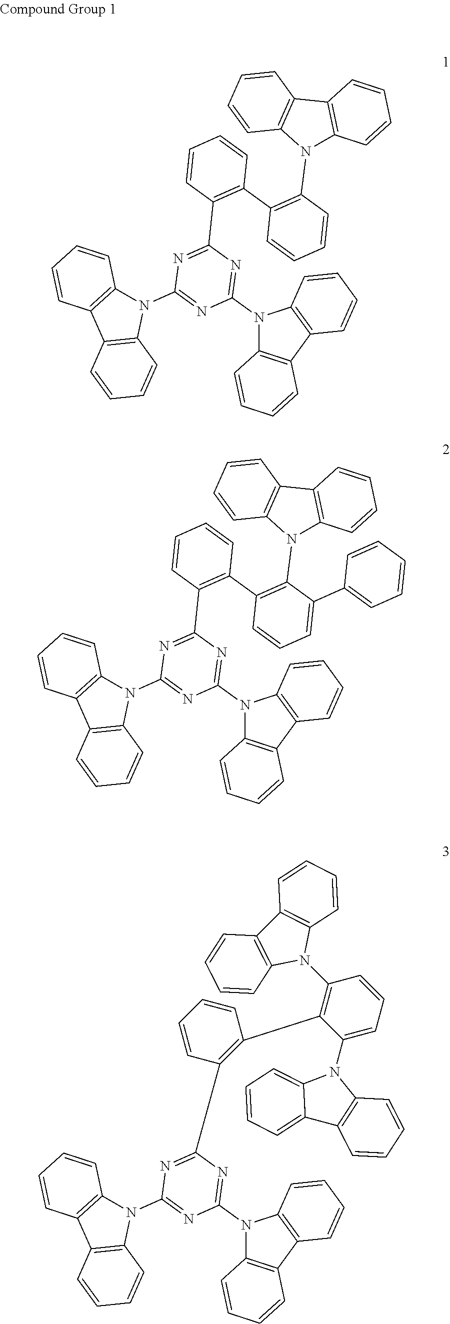 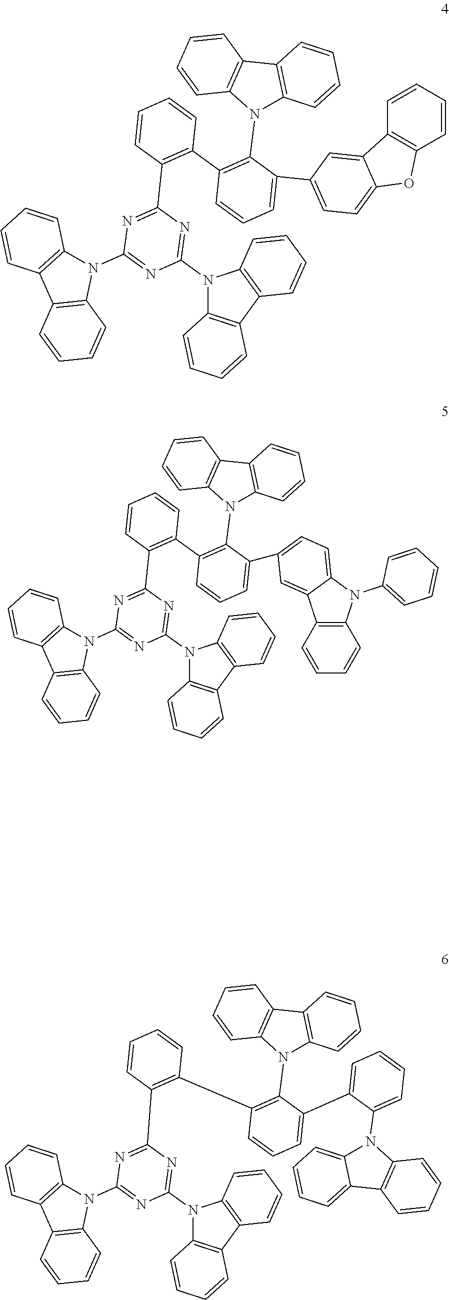 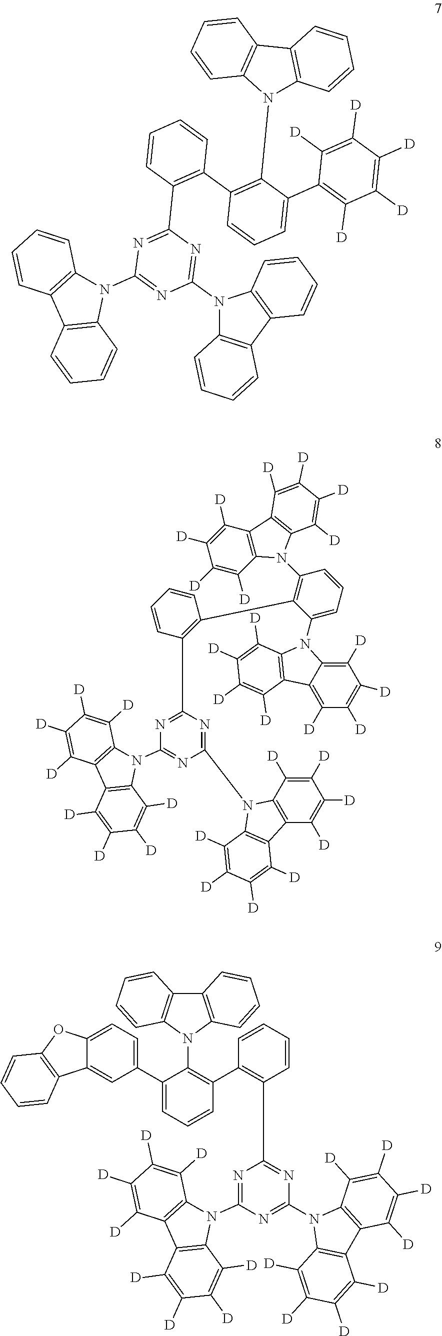 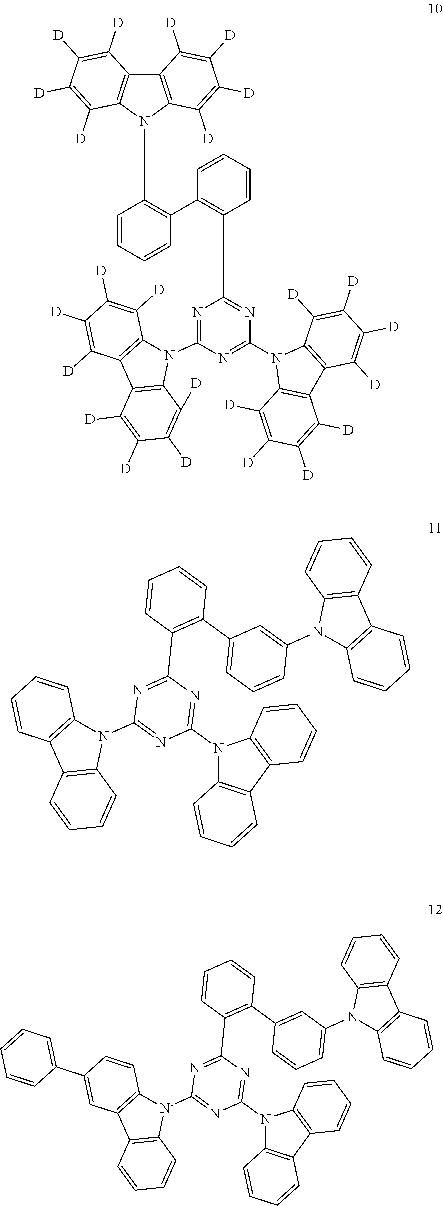 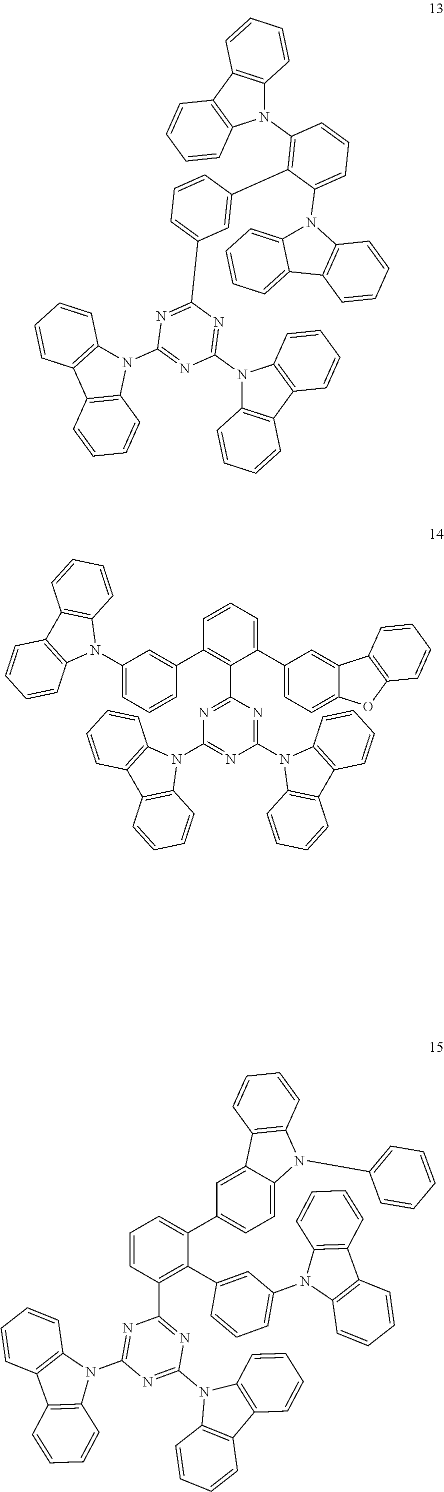 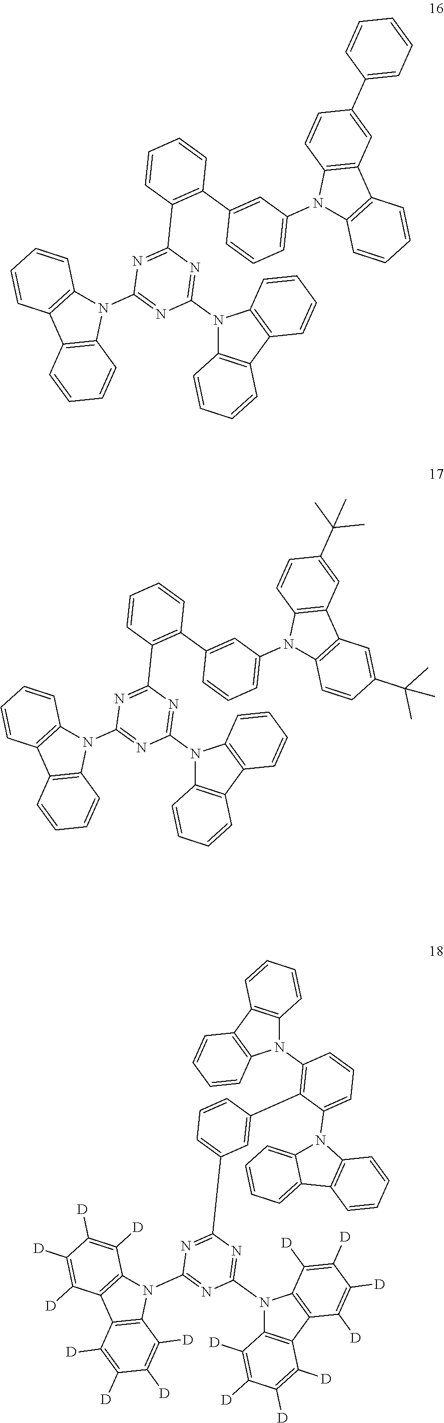 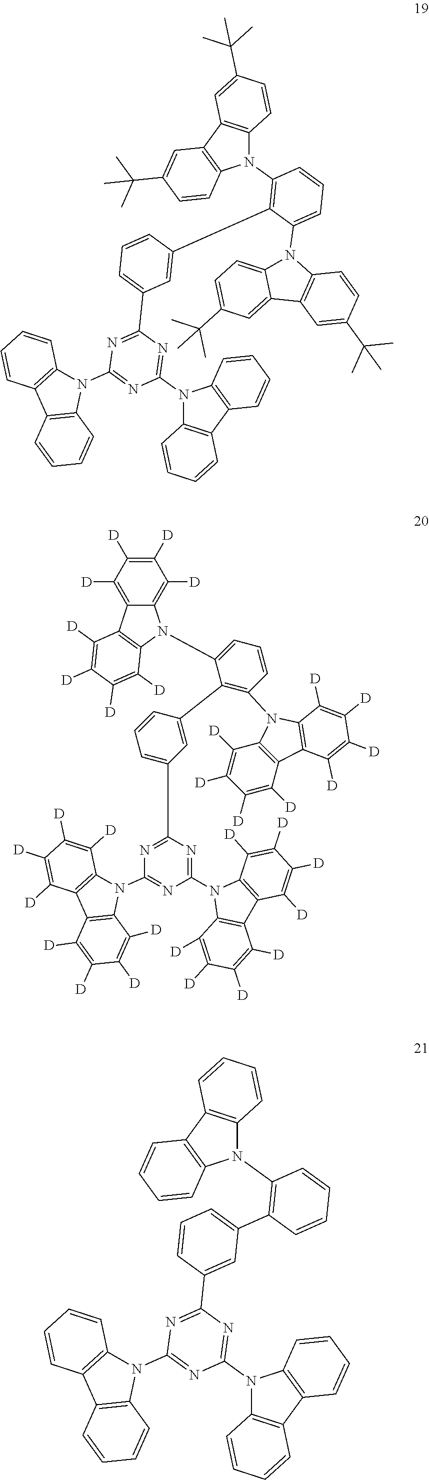 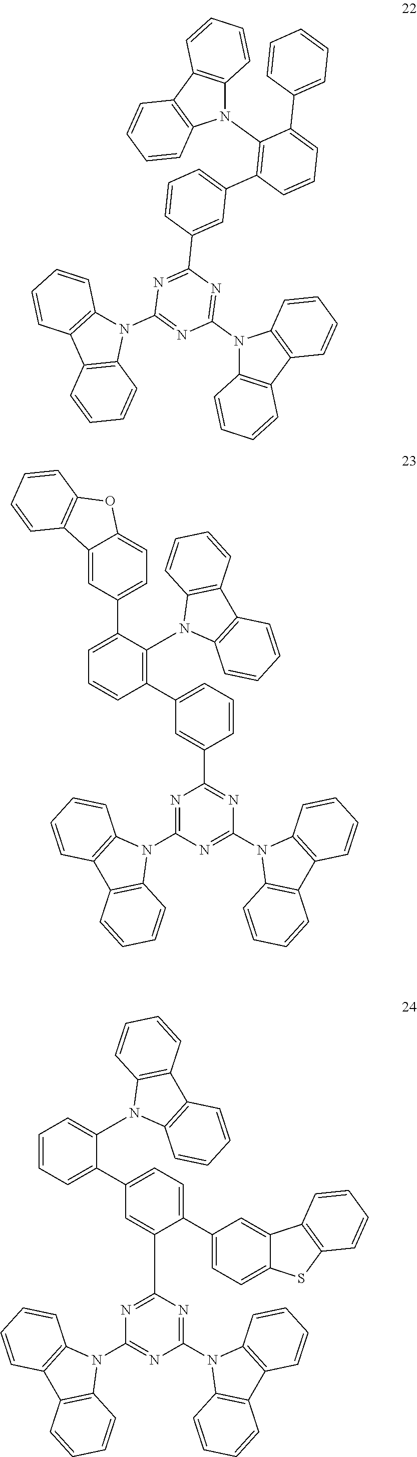 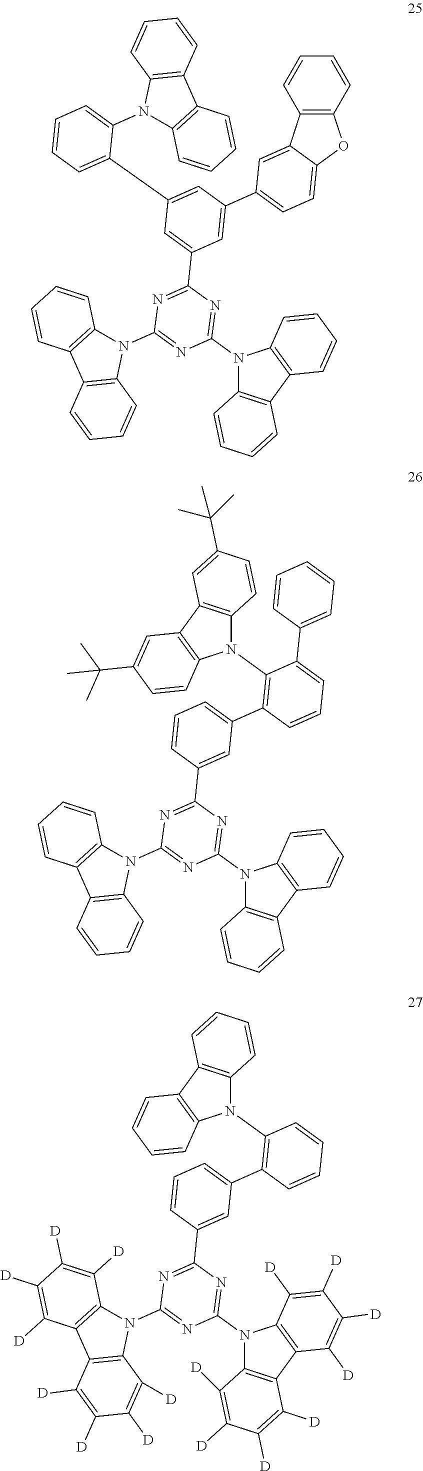 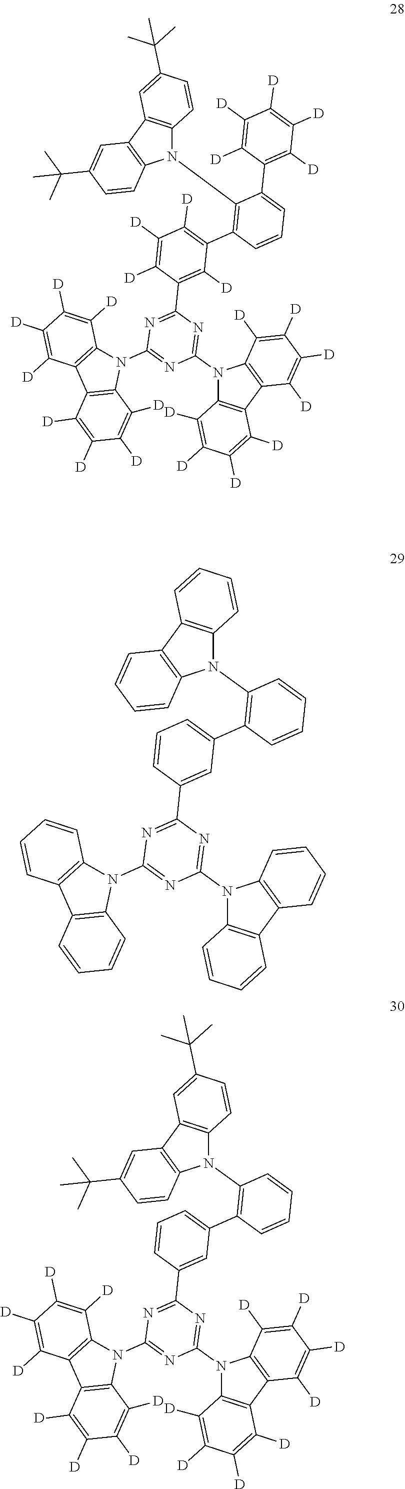 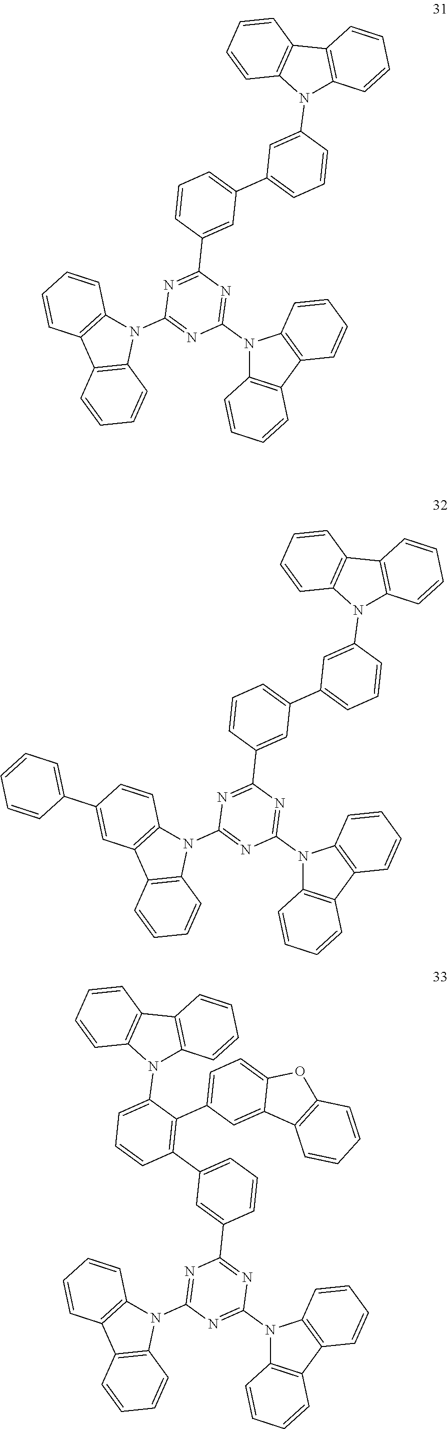 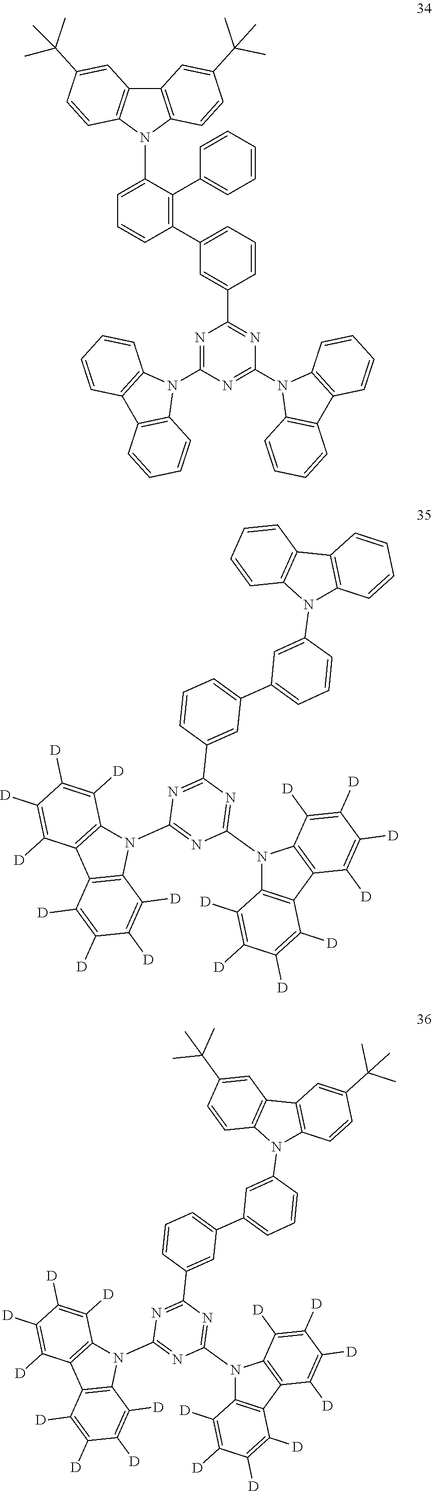 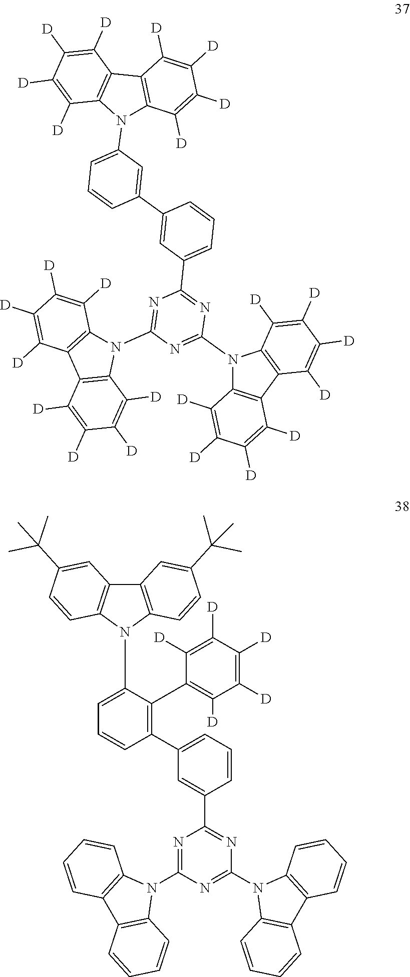 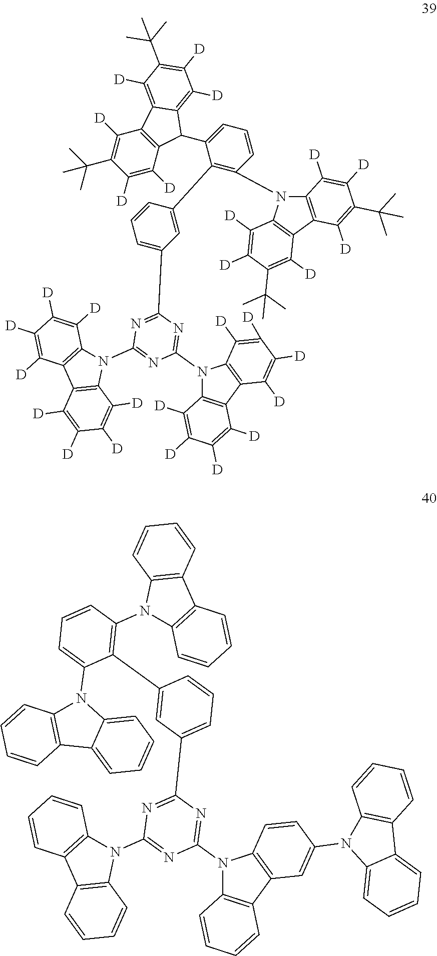 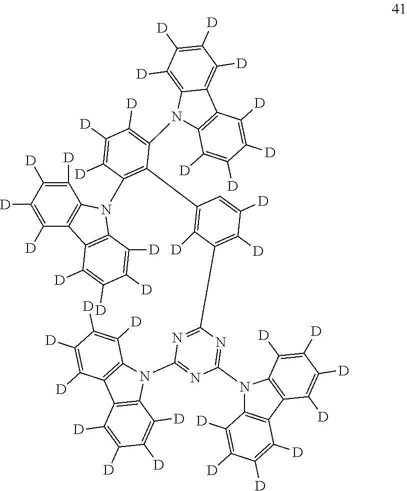
wherein, in Compound Group 1, D is a deuterium atom.

12. A nitrogen-containing compound represented by Formula 1: 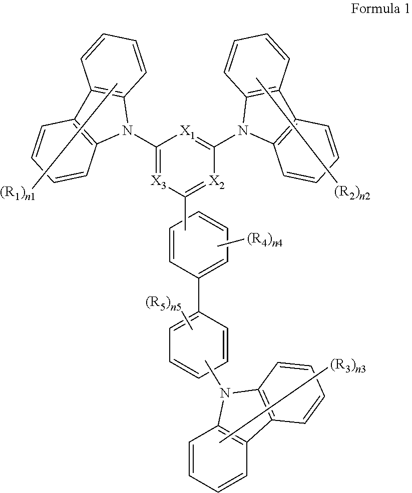
wherein, in Formula 1,
at least one among X₁ to X₃ is N, and the rest are each independently CR₆,
n1 to n3 are each independently an integer of 0 to 8,
n4 and n5 are each independently an integer of 0 to 4, and
R₁ to R₆ are each independently a hydrogen atom, a deuterium atom, a halogen atom, a hydroxy group, a cyano group, a nitro group, a substituted or unsubstituted amine group, a substituted or unsubstituted oxy group, a substituted or unsubstituted thio group, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted aryl group having 6 to 60 ring-forming carbon atoms, or a substituted or unsubstituted heteroaryl group having 2 to 60 ring-forming carbon atoms.

13. The nitrogen-containing compound of claim 12, wherein Formula 1 is represented by Formula 1-1 or Formula 1-2: 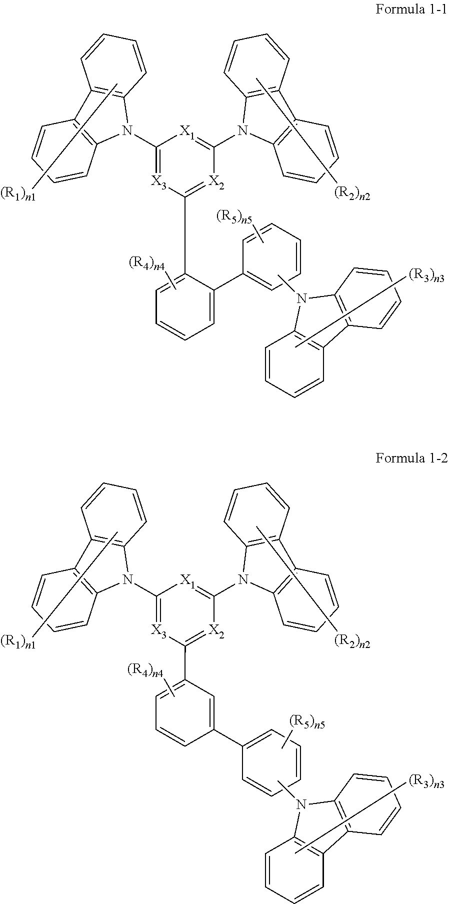
wherein, in Formula 1-1 and Formula 1-2,
X₁ to X₃, n1 to n5, and R₁ to R₅ are the same as defined in Formula 1.

14. The nitrogen-containing compound of claim 13, wherein Formula 1-1 is represented by Formula 1-1A or Formula 1-1B: 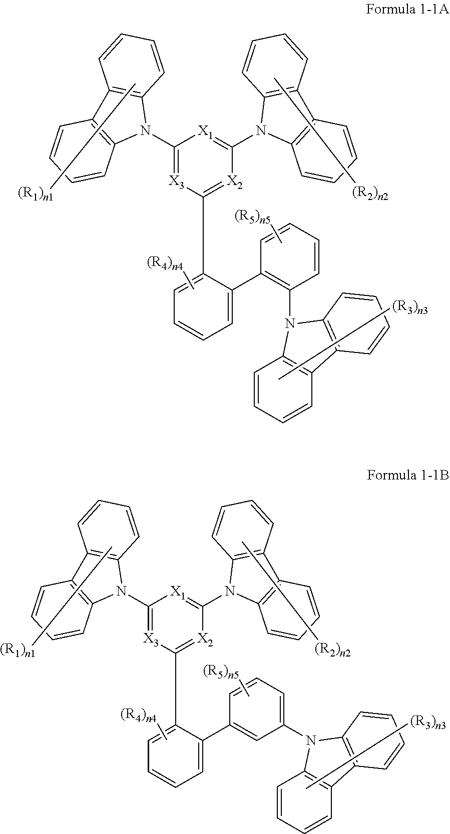
wherein, in Formula 1-1A and Formula 1-1B,
X₁ to X₃, n1 to n5, and R₁ to R₅ are the same as defined in Formula 1.

15. The nitrogen-containing compound of claim 13, wherein Formula 1-2 is represented by Formula 1-2A or Formula 1-2B: 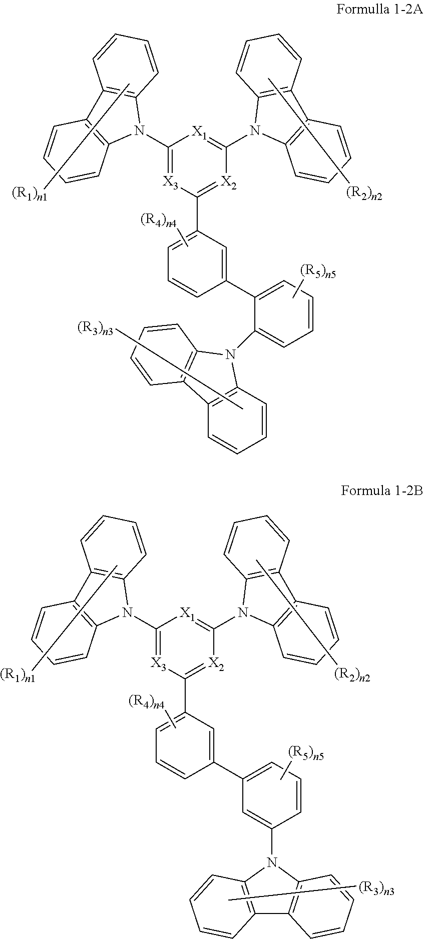
wherein, in Formula 1-2A and Formula 1-2B,
X₁ to X₃, n1 to n5, and R₁ to R₅ are the same as defined in Formula 1.

16. The nitrogen-containing compound of claim 12, wherein Formula 1 is represented by any one among Formula 1-3 to Formula 1-5: 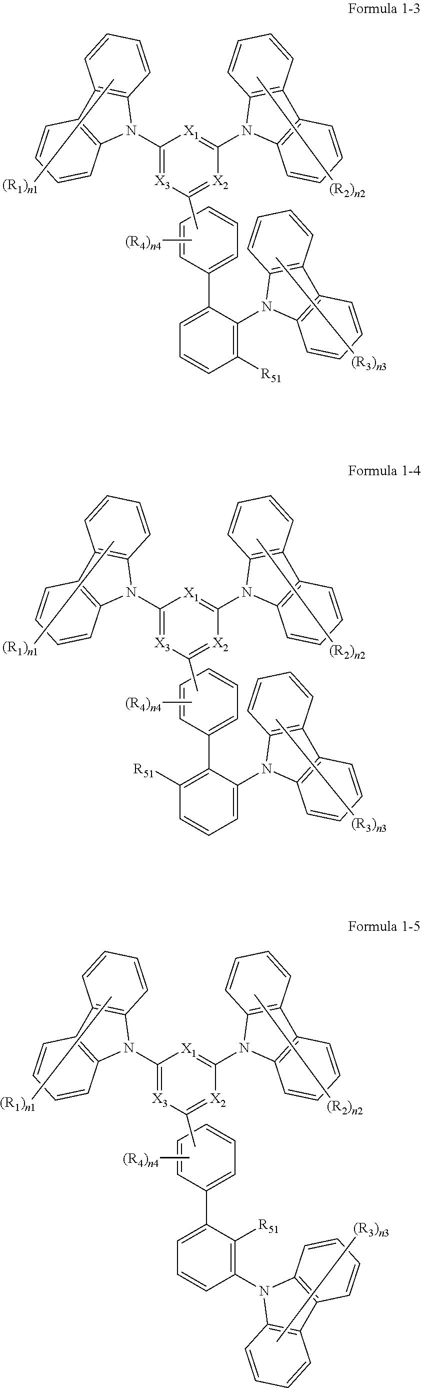
wherein, in Formula 1-3 to Formula 1-5,
R₅₁ is a substituted or unsubstituted phenyl group, a substituted or unsubstituted carbazole group, a substituted or unsubstituted dibenzofuran group, or a substituted or unsubstituted dibenzothiophene group, and
X₁ to X₃, n1 to n4, and R₁ to R₄ are the same as defined in Formula 1.

17. The nitrogen-containing compound of claim 12, wherein, in Formula 1, R₁ to R₆ are each independently a hydrogen atom, a deuterium atom, or represented by any one among R-1 to R-7: 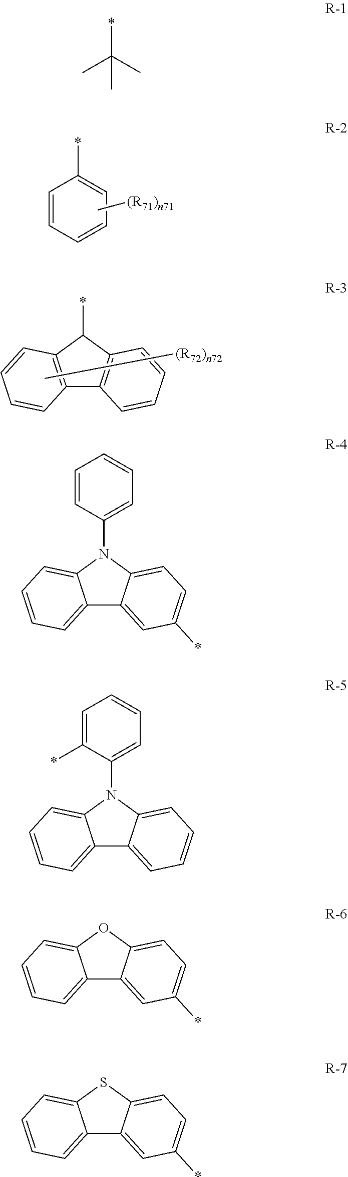
wherein, in R-2 and R-3,
n71 is an integer of 0 to 5,
n72 is an integer of 0 to 8, and
R₇₁ and R₇₂ are each independently a hydrogen atom, a deuterium atom, or an unsubstituted t-butyl group.

18. The nitrogen-containing compound of claim 12, wherein, in Formula 1, at least one among R₁ to R₅ is a deuterium atom or comprises a substituent containing a deuterium atom.

19. The nitrogen-containing compound of claim 12, wherein, in Formula 1, each of X₁ to X₃ is N.

20. The nitrogen-containing compound of claim 12, wherein Formula 1 is represented by any one among compounds of Compound Group 1: 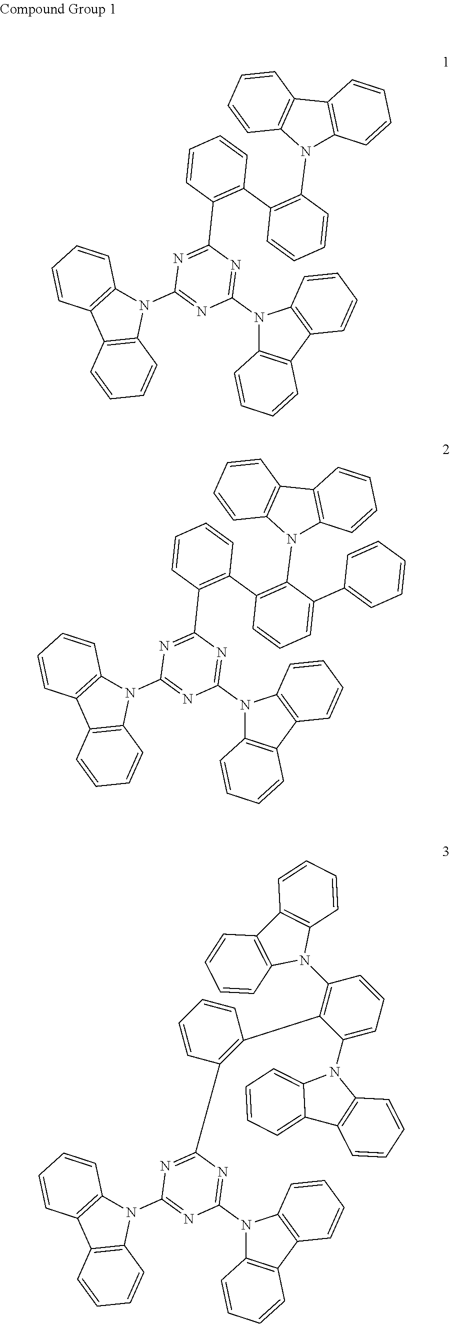 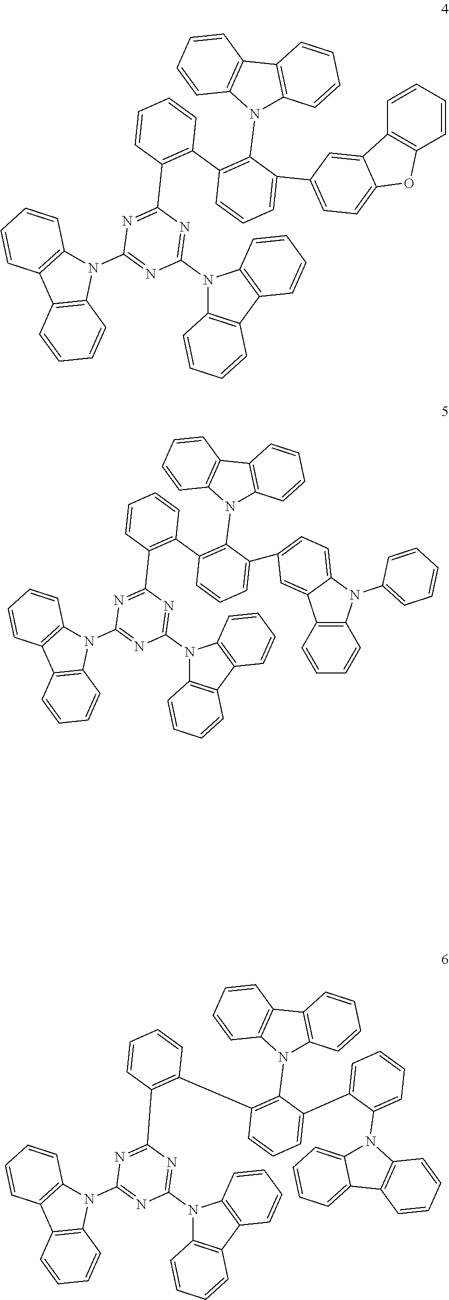 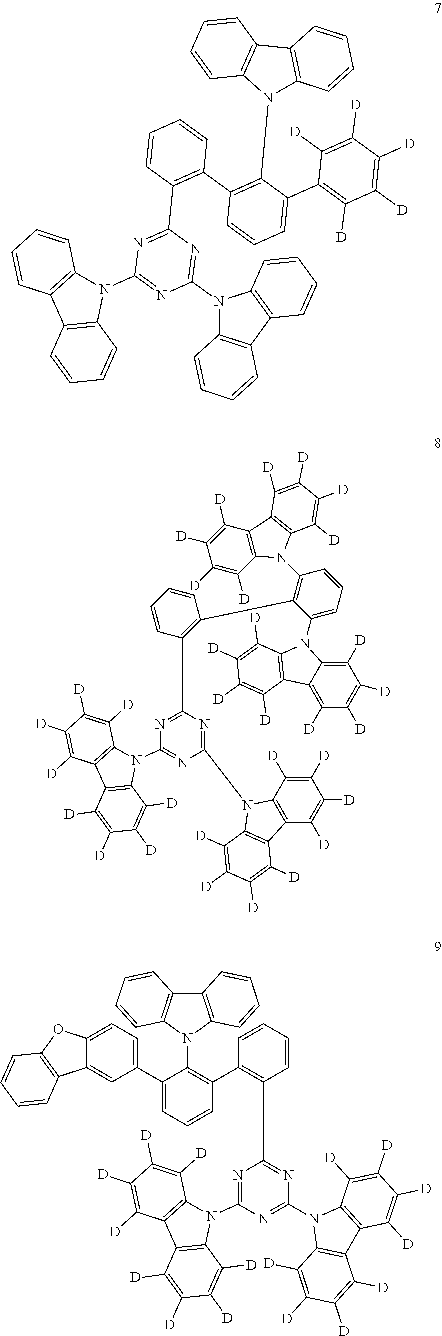 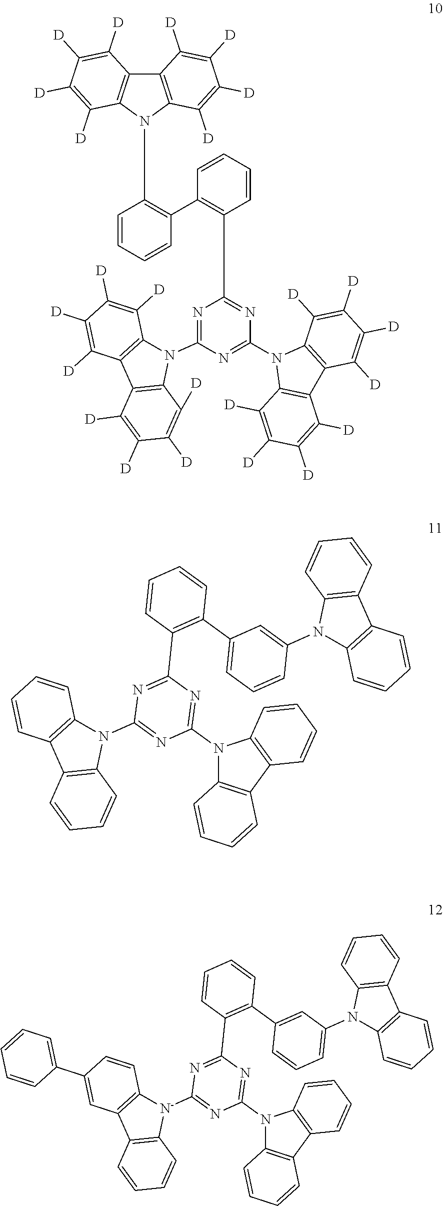 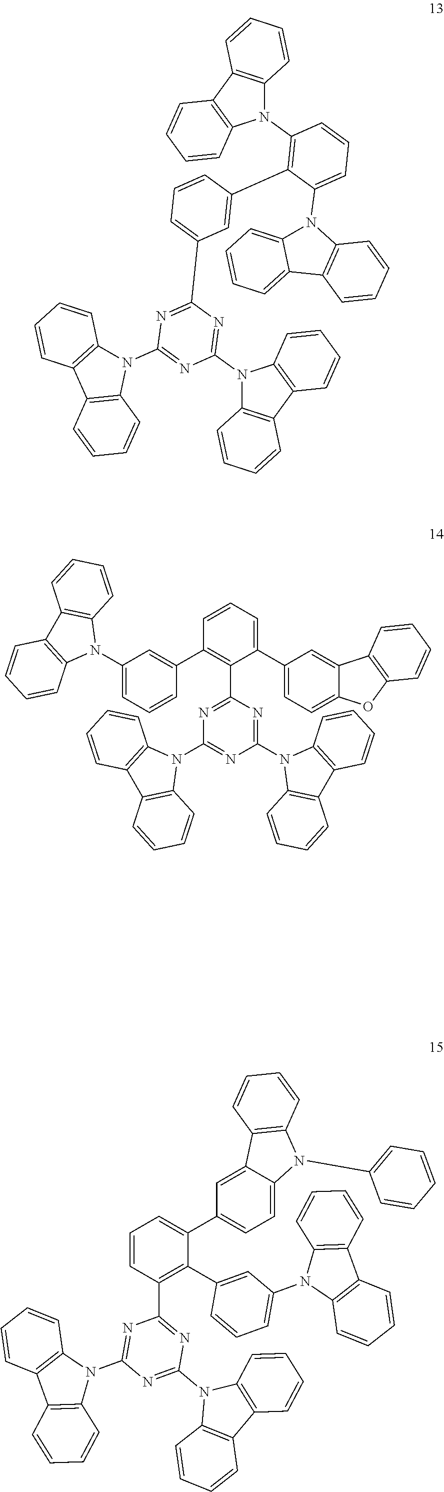 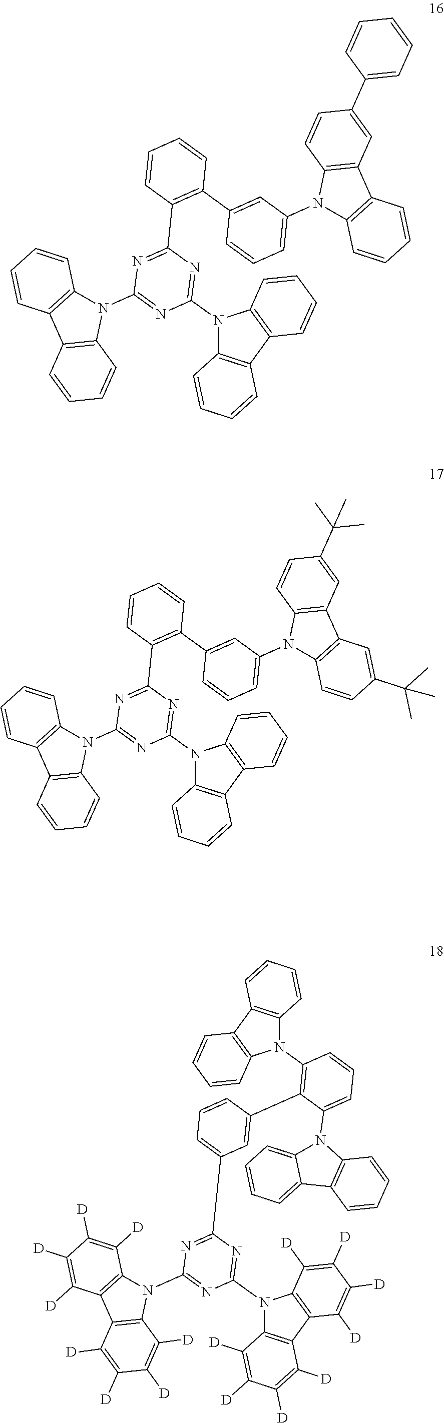 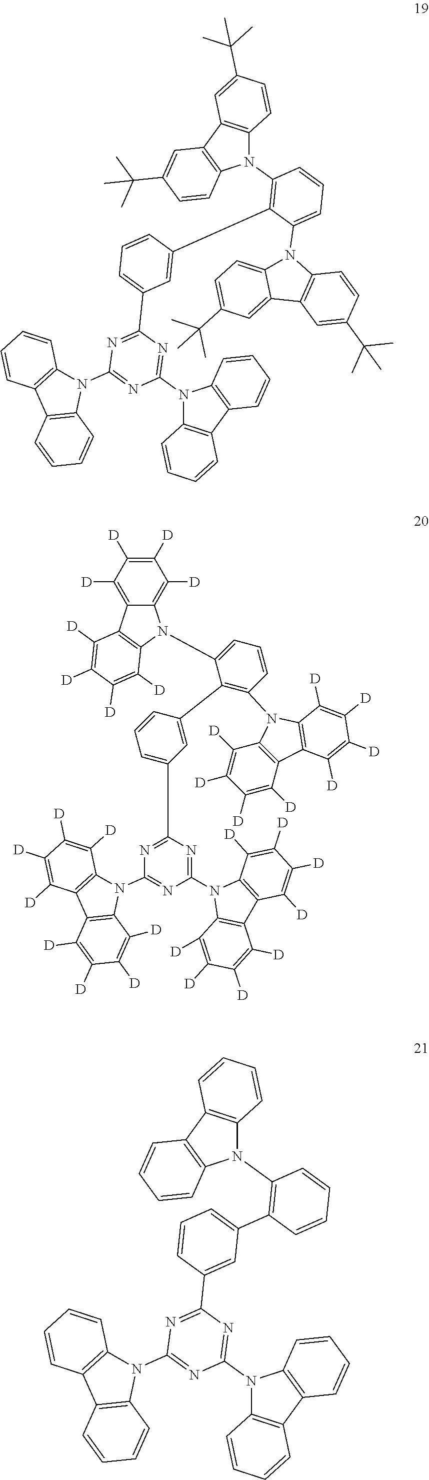 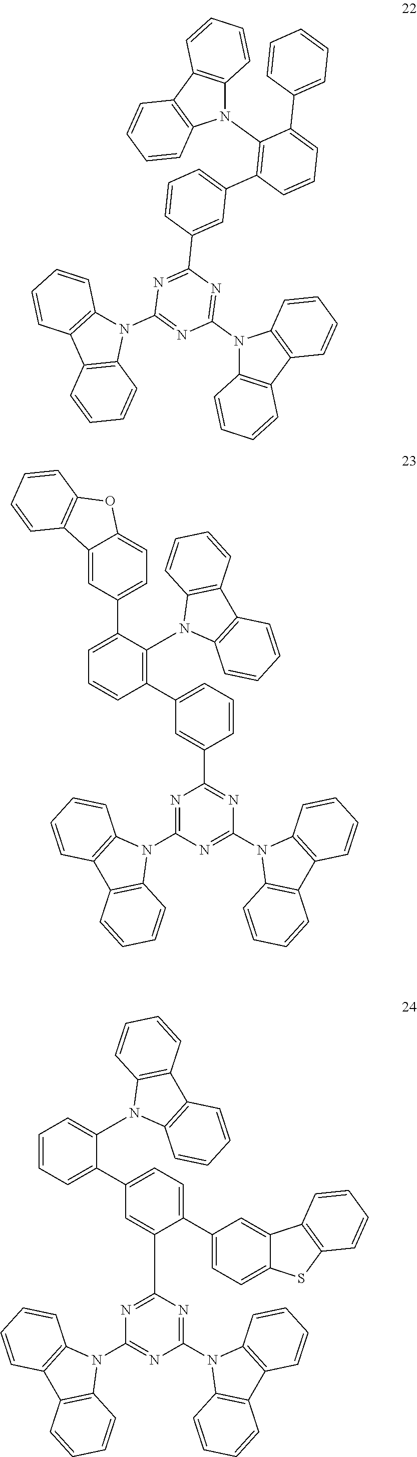 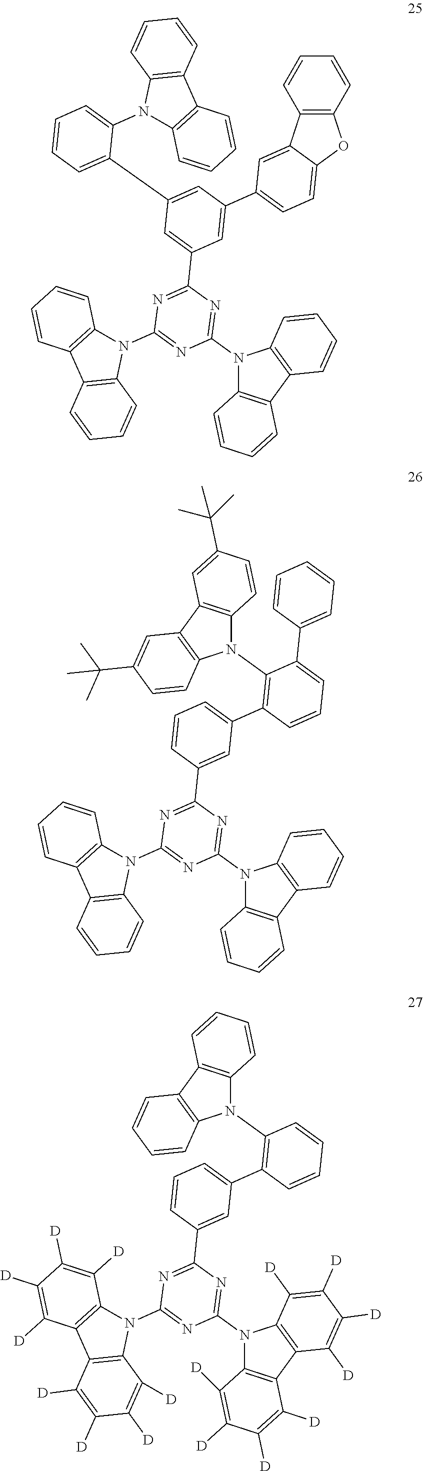 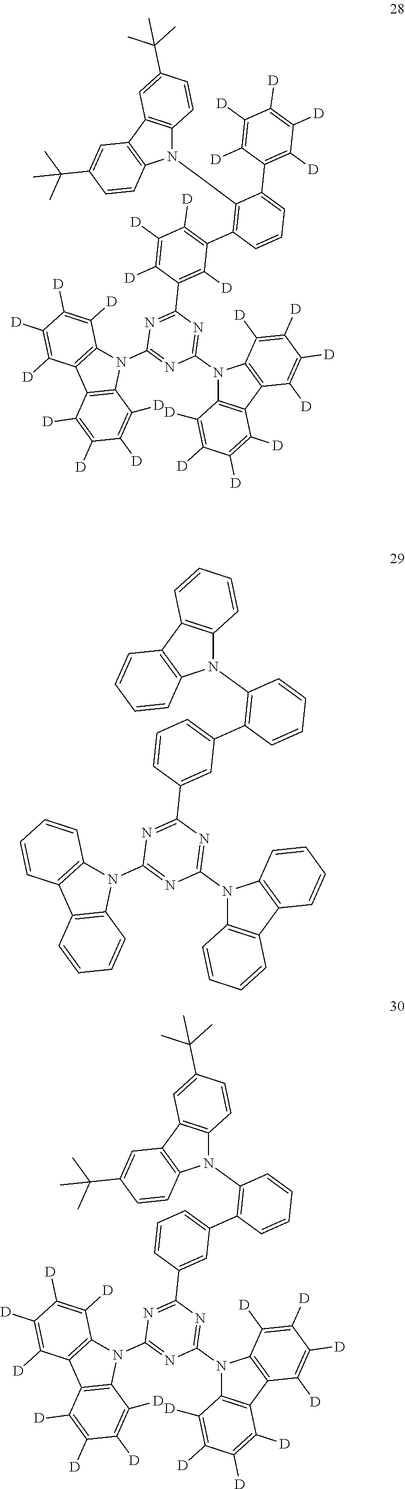 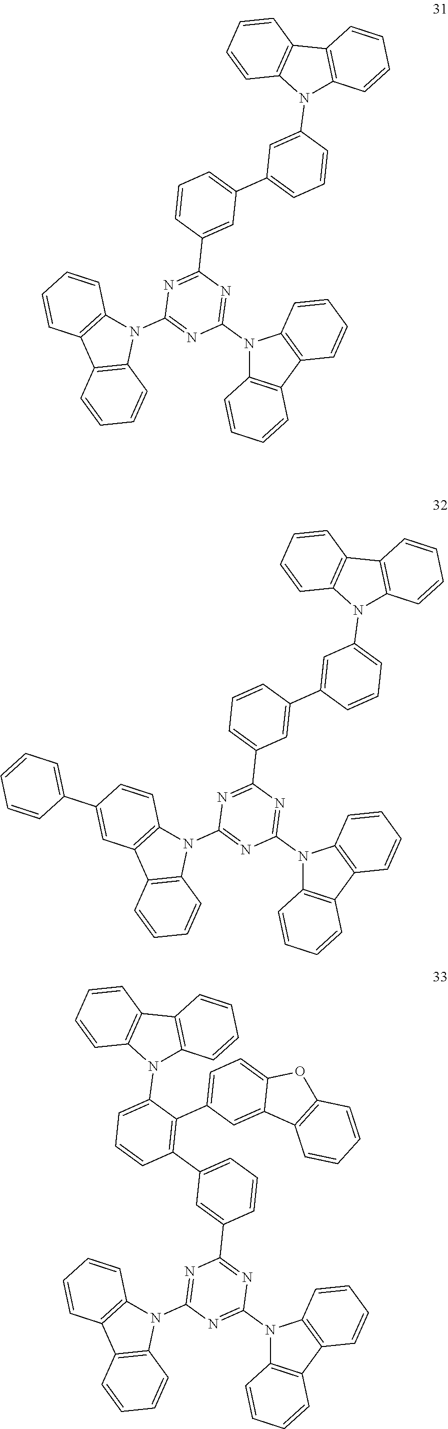 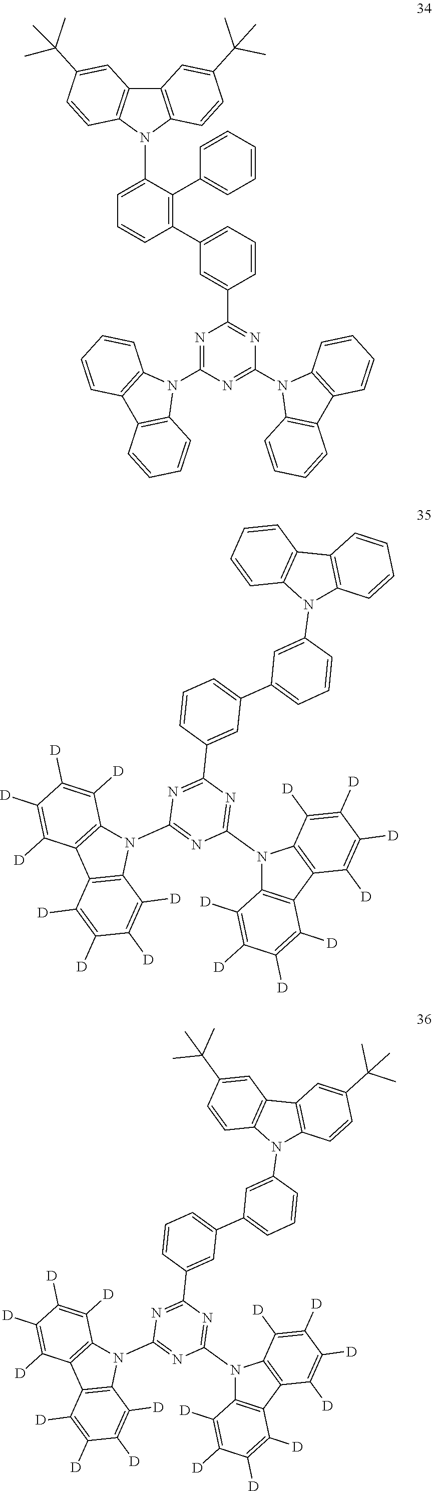 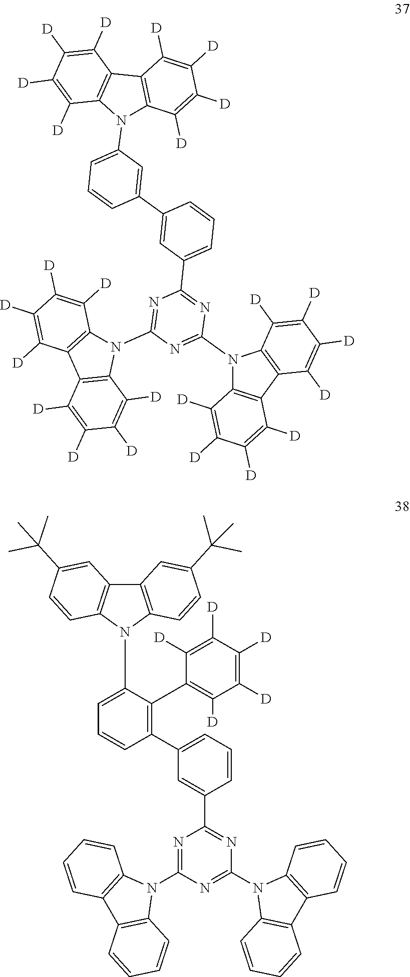 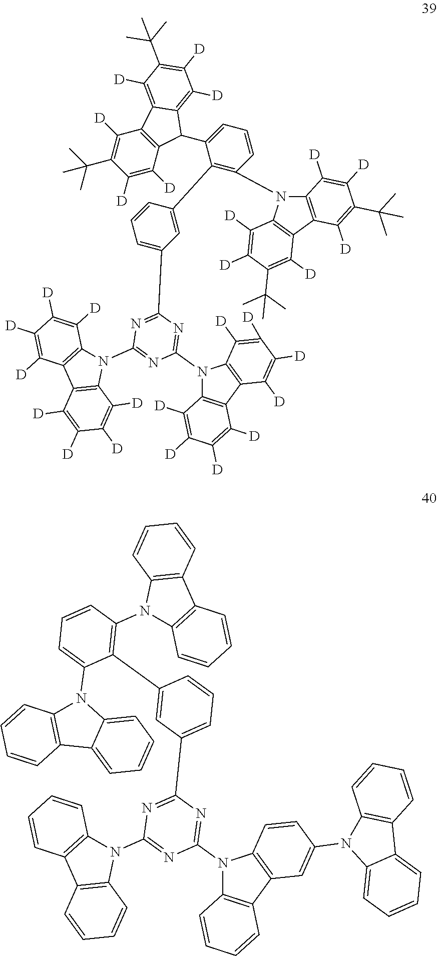 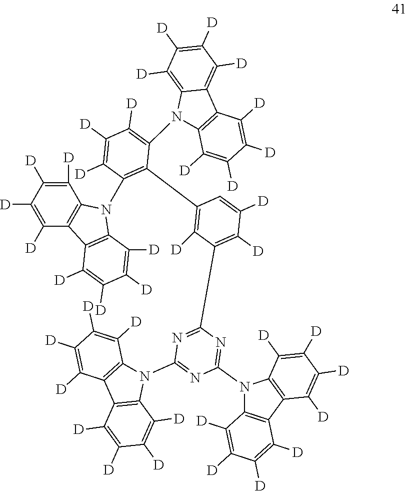
wherein, in Compound Group 1, D is a deuterium atom.
